(19) United States
(12) Patent Application Publication
(10) Pub. No.: US 2025/0372255 A1
(43) Pub. Date: Dec. 4, 2025

(54) MACHINE LEARNING MODEL TRAINED USING ARTIFICIAL CELL-FREE RNA (CFRNA) EXPRESSION DATA

(71) Applicant: BostonGene Corporation, Waltham, MA (US)

(72) Inventors: Boris Shpak, Yerevan (AM); Aleksandr Zaitsev, Yerevan (AM); Elena Bushmanova, Yerevan (AM); Andrei Shubin, Boston, MA (US); Maria Savchenko, Yerevan (AM); Daniil Litvinov, Basel (CH); Anastasiia Tarabarova, Yerevan (AM); Evgenii Bolshakov, Yerevan (AM); Anastasiia Radko, Yerevan (AM)

(73) Assignee: BostonGene Corporation, Waltham, MA (US)

(21) Appl. No.: 19/224,303

(22) Filed: May 30, 2025

Related U.S. Application Data
(60) Provisional application No. 63/654,427, filed on May 31, 2024.
(60) Provisional application No. 63/715,868, filed on Nov. 4, 2024.

(51) Int. Cl. G16H 50/20

(52) U.S. Cl. CPC G16H 50/20

(57) ABSTRACT

Some embodiments provide for a method of using a trained machine learning model to predict a characteristic of a subject, the method comprising: processing cfRNA expression data using the trained machine learning model to obtain an output indicative of the characteristic of the subject, wherein the trained machine learning model was trained using artificial cfRNA expression data, the artificial cfRNA expression data comprising a plurality of artificial cfRNA expression profiles, an artificial cfRNA expression profile having been generated by: generating a healthy expression profile component; generating a tumor expression profile component; and generating the artificial cfRNA expression profile using the healthy expression profile component and the tumor expression profile component.

### CROSS-REFERENCE TO RELATED APPLICATIONS

The present application claims the benefit of priority under 35 U.S.C. § 119 (e) of U.S. Provisional Patent Application Ser. No. 63/654,427 filed on May 31, 2024, under Attorney Docket No. B1462.70058US00, and entitled “MACHINE LEARNING MODEL TRAINED USING ARTIFICIAL CELL-FREE RNA (CFRNA) EXPRESSION DATA,” which is incorporated by reference herein in its entirety.

The present application also claims the benefit of priority under 35 U.S.C. 119 (e) of U.S. Provisional Patent Application Ser. No. 63/715,868 filed on Nov. 4, 2024, under Attorney Docket No. B1462.70058US01, and entitled “MACHINE LEARNING TECHNIQUES FOR ANALYZING CELL-FREE RNA (CFRNA),” which is incorporated by reference herein in its entirety.

### BACKGROUND

Cell-free RNA (cfRNA) is RNA that is present in biological fluids (e.g., blood) independent of cells. cfRNA can include RNA that is shed by both tumor and non-tumor cells.

### SUMMARY

Some aspects provide for a method of using a trained machine learning model to predict a characteristic of a subject based on cell-free RNA (cfRNA) expression data previously-obtained from a blood sample from the subject, the method comprising: using at least one computer hardware processor to perform: obtaining the cfRNA expression data; and processing the cfRNA expression data using the trained machine learning model to obtain an output indicative of the characteristic of the subject, wherein the trained machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more tissue types; and generating the healthy expression profile component by combining the plurality of RNA expression profiles; generating a tumor expression profile component; and generating the artificial cfRNA expression profile using the healthy expression profile component and the tumor expression profile component.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of using a trained machine learning model to predict a characteristic of a subject based on cell-free RNA (cfRNA) expression data previously-obtained from a blood sample from the subject, the method comprising: obtaining the cfRNA expression data; and processing the cfRNA expression data using the trained machine learning model to obtain an output indicative of the characteristic of the subject, wherein the trained machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more tissue types; and generating the healthy expression profile component by combining the plurality of RNA expression profiles; generating a tumor expression profile component; and generating the artificial cfRNA expression profile using the healthy expression profile component and the tumor expression profile component.

Some aspects provide for at least one non-transitory computer-readable storage medium storing processor-executable instructions that, when executed by at least one computer hardware processor, causes the at least one computer hardware processor to perform a method of using a trained machine learning model to predict a characteristic of a subject based on cell-free RNA (cfRNA) expression data previously-obtained from a blood sample from the subject, the method comprising: obtaining the cfRNA expression data; and processing the cfRNA expression data using the trained machine learning model to obtain an output indicative of the characteristic of the subject, wherein the trained machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more tissue types; and generating the healthy expression profile component by combining the plurality of RNA expression profiles; generating a tumor expression profile component; and generating the artificial cfRNA expression profile using the healthy expression profile component and the tumor expression profile component.

Some aspects provide for a method of predicting a characteristic of a subject based on cell-free RNA (cfRNA) expression data previously-obtained from a biological fluid sample (e.g., a blood sample) from the subject, the method comprising: using at least one computer hardware processor to perform: obtaining the cfRNA expression data; and processing the cfRNA expression data using a machine learning model trained to process cfRNA expression data from a subject and produce an output indicative of the characteristic of the subject, wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component by: receiving a tumor expression profile from a tumor sample from a subject having cancer; and generating the tumor expression profile component using the tumor expression profile component; and generating the artificial cfRNA expression profile by combining the healthy expression profile component and the tumor expression profile component.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of predicting a characteristic of a subject based on cell-free RNA (cfRNA) expression data previously-obtained from a biological fluid sample (e.g., a blood sample) from the subject, the method comprising: obtaining the cfRNA expression data; and processing the cfRNA expression data using a machine learning model trained to process cfRNA expression data from a subject and produce an output indicative of the characteristic of the subject, wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component by: receiving a tumor expression profile from a tumor sample from a subject having cancer; and generating the tumor expression profile component using the tumor expression profile component; and generating the artificial cfRNA expression profile by combining the healthy expression profile component and the tumor expression profile component.

Some aspects provide for at least one non-transitory computer-readable storage medium storing processor-executable instructions that, when executed by at least one computer hardware processor, causes the at least one computer hardware processor to perform a method of predicting a characteristic of a subject based on cell-free RNA (cfRNA) expression data previously-obtained from a biological fluid sample (e.g., a blood sample) from the subject, the method comprising: obtaining the cfRNA expression data; and processing the cfRNA expression data using a machine learning model trained to process cfRNA expression data from a subject and produce an output indicative of the characteristic of the subject, wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component by: receiving a tumor expression profile from a tumor sample from a subject having cancer; and generating the tumor expression profile component using the tumor expression profile component; and generating the artificial cfRNA expression profile by combining the healthy expression profile component and the tumor expression profile component.

Embodiments of any of the above aspects may have one or more of the following features.

In some embodiments, the trained machine learning model is a machine learning model that has been trained to predict whether the subject has cancer.

In some embodiments, the trained machine learning model is a machine learning model that has been trained to take as input a cfRNA expression profile for a subject and provide as output a prediction of whether the subject has cancer. In some embodiments, the output indicative of the characteristic of the subject comprises an indication of whether the subject has cancer.

Some embodiment further comprise when the output of the trained machine learning model indicates that the subject has the cancer, generating a recommendation to perform a diagnostic test.

Some embodiments further comprise: when the output of the trained machine learning model indicates that the subject has the cancer, performing the diagnostic test.

In some embodiments, the cancer is breast cancer, and wherein the diagnostic test comprises a mammography and/or a biopsy.

Some embodiments further comprise: when the output of the trained machine learning model indicates that the subject does not have the cancer, generating a recommendation to stop administering a therapy to the subject.

In some embodiments, the trained machine learning model is a machine learning model that has been trained to predict whether the subject has liver metastasis.

In some embodiments, the trained machine learning model is a machine learning model that has been trained to take as input a cfRNA expression profile for a subject and provide as output a prediction of whether the subject has liver metastasis. In some embodiments, output indicative of the characteristic of the subject comprises an indication of whether the subject has liver metastasis.

Some embodiments further comprise when the output of the trained machine learning model indicates that the subject has liver metastasis, generating a recommendation to perform an ultrasound and/or a biopsy.

Some embodiments further comprise when the output of the trained machine learning model indicates that the subject has liver metastasis, performing an ultrasound and/or a biopsy.

In some embodiments, the trained machine learning model is a machine learning model that has been trained to predict a fraction of malignant B cells relative to total number of B cells in the biological fluid sample from the subject.

In some embodiments, the trained machine learning model is a machine learning model that has been trained to take as input a cfRNA expression profile for a subject and provide as output a prediction of the fraction of malignant B cells. In some embodiments, the output indicative of the characteristic of the subject comprises an indication of the fraction of malignant B cells.

Some embodiments further comprise generating a recommendation to administer an anti-cancer treatment based on the fraction of malignant B cells.

Some embodiments further comprise administering an anti-cancer treatment based on the fraction of malignant B cells.

Some embodiments further comprise determining, based on the fraction of malignant B cells, whether the subject has chronic lymphocytic leukemia (CLL).

In some embodiments, the trained machine learning model is a machine learning model that has been trained to predict a PD-1 status for the subject, wherein the PD-1 status is indicative of whether PDCD1 is expressed in tumor cells of the subject.

In some embodiments, the trained machine learning model is a machine learning model that has been trained to take as input a cfRNA expression profile for a subject and provide as output a prediction of the PD-1 status. In some embodiments, the output indicative of the characteristic of the subject comprises an indication of the PD-1 status.

Some embodiments further comprise generating a recommendation to administer an anti-cancer treatment based on the PD-1 status.

Some embodiments further comprise administering an anti-cancer treatment based on the PD-1 status.

In some embodiments, the artificial cfRNA expression data comprises: a first plurality of artificial cfRNA expression profiles generated using a first plurality of healthy expression profile components, and a second plurality of artificial cfRNA expression profiles generated using a second plurality of healthy expression profile components and a plurality of tumor expression profile components, the plurality of tumor expression profile components having been generated using tumor expression profiles from tumor samples obtained from subjects having cancer.

In some embodiments, the first plurality of artificial cfRNA expression profiles correspond to the first plurality of healthy expression profile components, wherein a healthy expression profile component is generated by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles.

In some embodiments, the trained machine learning model is a machine learning model that has been trained to predict whether a subject has cancer using training data comprising: the first plurality of artificial cfRNA expression profiles generated using the first plurality of healthy expression profile components, and the second plurality of artificial cfRNA expression profiles generated using the second plurality of healthy expression profile components and the plurality of tumor expression profile components.

In some embodiments, each artificial cfRNA expression profile in the training data is associated with a ground truth label. In some embodiments, the first plurality of artificial cfRNA expression profiles are associated with a label indicating that the subject does not have the cancer, and the second plurality of artificial cfRNA expression profiles are associated with a label indicating that the subject has the cancer.

In some embodiments, the artificial cfRNA expression data comprises: a first plurality of artificial cfRNA expression profiles generated using a first plurality of healthy expression profile components and a first plurality of tumor expression profile components, the first plurality of healthy expression profile components having been generated using at least one RNA expression profile previously-obtained from liver tissue, and a second plurality of artificial cfRNA expression profiles generated using a second plurality of healthy expression profile components and a plurality of tumor expression profile components, the second plurality of healthy expression profile components having been generated without using at least one RNA expression profile previously-obtained from liver tissue.

In some embodiments, the trained machine learning model is a machine learning model that has been trained to predict whether the subject has liver metastasis using training data comprising: the first plurality of artificial cfRNA expression profiles, and the second plurality of artificial cfRNA expression profiles.

In some embodiments, each artificial cfRNA expression profile in the training data is associated with a ground truth label. In some embodiments, the first plurality of artificial cfRNA expression profiles are associated with a label indicating that the subject has the liver metastasis, and the second plurality of artificial cfRNA expression profiles are associated with a label indicating that the subject does not have the liver metastasis.

In some embodiments, the artificial cfRNA expression data comprises: a first plurality of artificial cfRNA expression profiles generated using a first plurality of healthy expression profile components and a first plurality of tumor expression profile components, the first plurality of tumor expression profile components having been generated using tumor expression profiles from tumor samples that express PDCD1 (PDCD1+), and a second plurality of artificial cfRNA expression profiles generated using a second plurality of healthy expression profile components and a second plurality of tumor expression profile components, the second plurality of tumor expression profile components having been generated using tumor expression profiles from tumor samples that do not express PDCD1 (PDCD1−).

In some embodiments, the trained machine learning model is a machine learning model that has been trained to predict a PD-1 status of the subject using training data comprising: the first plurality of artificial cfRNA expression profiles, and the second plurality of artificial cfRNA expression profiles.

In some embodiments, each artificial cfRNA expression profile in the training data is associated with a ground truth label. In some embodiments, the first plurality of artificial cfRNA expression profiles are associated with a label indicating that the artificial cfRNA expression profile represents samples that express PDCD1, and the second plurality of artificial cfRNA expression profiles are associated with a label indicating that the artificial cfRNA expression profile represents samples that do not express PDCD1.

In some embodiments the trained machine learning model is a machine learning model that has been trained to predict a fraction of malignant B cells relative to a total number of B cells in the biological fluid sample from the subject using training data comprising the plurality of artificial expression profiles.

In some embodiments, each artificial cfRNA expression profile in the training data is associated with a ground truth label indicating the fraction of malignant B cells corresponding to the particular artificial cfRNA expression profile.

In some embodiments, the plurality of artificial cfRNA expression profiles comprise at least 100 artificial cfRNA expression profiles, at least 250 artificial cfRNA expression profiles, at least 500 artificial cfRNA expression profiles, at least 1,000 artificial cfRNA expression profiles, at least 1,500 artificial cfRNA expression profiles, at least 2,000 artificial cfRNA expression profiles, at least 2,500 artificial cfRNA expression profiles, at least 3,000 artificial cfRNA expression profiles, at least 4,000 artificial cfRNA expression profiles, at least 5,000 artificial cfRNA expression profiles, or at least 10,000 artificial cfRNA expression profiles.

In some embodiments, the trained machine learning model is a decision tree model, a gradient boosted decision tree model, a linear regression model, a non-linear regression model, a support vector machine, a Gaussian mixture model, a random forest model, or a neural network model.

Some embodiments further comprise obtaining the cfRNA expression data from the biological fluid sample from the subject by sequencing the biological fluid sample. Thus, also described herein are methods comprising: obtaining cfRNA expression data from a blood sample previously obtained from a subject by sequencing the blood sample, and performing a computer-implemented method of predicting a characteristic of a subject based on cell-free RNA (cfRNA) expression data as described herein.

In some embodiments, generating the healthy expression profile component by combining the plurality of RNA expression profiles comprises combining the plurality of RNA expression profiles and a cfRNA expression profile previously-obtained from a biological fluid sample from a healthy subject.

Some embodiments further comprise training the trained machine learning model to predict the characteristic of the subject using the artificial cfRNA expression data.

Some embodiments further comprise generating the artificial cfRNA expression data by generating each particular artificial cfRNA expression profile of the plurality of artificial cfRNA expression profiles.

In some embodiments, combining the plurality of RNA expression profiles comprises determining a weighted sum of the plurality of RNA expression profiles.

In some embodiments, combining the healthy expression profile component and the tumor expression profile component comprises determining a weighted sum of the healthy expression profile component and the tumor expression profile component.

In some embodiments, the tumor expression profile comprises a plurality of counts for a respective plurality of genes. In some embodiments, the counts are counts of reads from RNA sequencing. In some embodiments, generating the tumor expression profile component using the tumor expression profile component comprises: determining, using the plurality of counts, a plurality of sampling probabilities including a respective sampling probability for each of the plurality of genes; sampling a plurality of reads from a multinomial distribution using at least some of the plurality of sampling probabilities, each of the plurality of reads corresponding to a gene of the plurality of genes; and generating the tumor expression profile component by summing, for each particular gene of the plurality of genes, a number of sampled reads corresponding to the particular gene.

In some embodiments, the plurality of reads comprises a number of reads determined by sampling a value from a uniform distribution.

Some aspects provide for a method of training a machine learning model to predict a characteristic of a subject based on cell-free RNA (cfRNA) expression data previously-obtained from a blood sample from the subject, the method comprising: using at least one computer hardware processor to perform: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject (e.g. predicting whether the subject has the characteristic) using the artificial cfRNA expression data, a particular artificial cfRNA expression profile of the first plurality of artificial cfRNA expression profiles (e.g. one or more or each of the artificial cfRNA expression profiles of the first plurality of artificial cfRNA expression profiles) or the second plurality of artificial cfRNA expression profiles (e.g. one or more or each of the artificial cfRNA expression profiles of the second plurality of artificial cfRNA expression profiles) having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the particular artificial cfRNA expression profile using the healthy expression profile component and the tumor expression profile component.

Some aspects provide for a method of training a machine learning model to predict a characteristic of a subject based on cell-free RNA (cfRNA) expression data previously-obtained from a biological fluid sample (e.g., a blood sample) from the subject, the method comprising: using at least one computer hardware processor to perform: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject (e.g. predicting whether the subject has the characteristic) using the artificial cfRNA expression data, a particular artificial cfRNA expression profile of the first plurality of artificial cfRNA expression profiles (e.g. one or more or each of the artificial cfRNA expression profiles of the first plurality of artificial cfRNA expression profiles) having been generated by: generating a first healthy expression profile component by: receiving a first plurality of RNA expression profiles previously-obtained from biological samples from healthy subjects, the first plurality of RNA expression profiles including a respective RNA expression profile for each of one or more cell types and/or each of one or more types of cell-containing samples; and generating the first healthy expression profile component by combining the first plurality of RNA expression profiles; generating a first tumor expression profile component; and generating the particular artificial cfRNA expression profile by combining the first healthy expression profile component and the first tumor expression profile component.

Methods according to the present aspect may have any of the features described herein in relation to methods of using a trained machine learning model to predict a characteristic of a subject based on cell-free RNA (cfRNA) expression data previously-obtained from a blood sample from the subject. Further, methods of using a trained machine learning model to predict a characteristic of a subject based on cell-free RNA (cfRNA) expression data previously-obtained from a blood sample from the subject as described herein may comprise training a machine learning model as described according to the present aspects.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of training a machine learning model to predict a characteristic of a subject based on cell-free RNA (cfRNA) expression data previously-obtained from a blood sample from the subject, the method comprising: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject using the artificial cfRNA expression data, a particular artificial cfRNA expression profile of the first plurality of artificial cfRNA expression profiles or the second plurality of artificial cfRNA expression profiles having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the particular artificial cfRNA expression profile using the healthy expression profile component and the tumor expression profile component.

Some aspects provide for at least one non-transitory computer-readable storage medium storing processor-executable instructions that, when executed by at least one computer hardware processor, causes the at least one computer hardware processor to perform a method of training a machine learning model to predict a characteristic of a subject based on cell-free RNA (cfRNA) expression data previously-obtained from a blood sample from the subject, the method comprising: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject using the artificial cfRNA expression data, a particular artificial cfRNA expression profile of the first plurality of artificial cfRNA expression profiles or the second plurality of artificial cfRNA expression profiles having been generated by: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the particular artificial cfRNA expression profile using the healthy expression profile component and the tumor expression profile component.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of training a machine learning model to predict a characteristic of a subject based on cell-free RNA (cfRNA) expression data previously-obtained from a biological fluid sample (e.g., a blood sample) from the subject, the method comprising: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject using the artificial cfRNA expression data, a particular artificial cfRNA expression profile of the first plurality of artificial cfRNA expression profiles having been generated by: generating a first healthy expression profile component by: receiving a first plurality of RNA expression profiles previously-obtained from biological samples from healthy subjects, the first plurality of RNA expression profiles including a respective RNA expression profile for each of one or more cell types and/or each of one or more types of cell-containing samples; and generating the first healthy expression profile component by combining the first plurality of RNA expression profiles; generating a first tumor expression profile component; and generating the particular artificial cfRNA expression profile by combining the first healthy expression profile component and the first tumor expression profile component.

Some aspects provide for at least one non-transitory computer-readable storage medium storing processor-executable instructions that, when executed by at least one computer hardware processor, causes the at least one computer hardware processor to perform a method of training a machine learning model to predict a characteristic of a subject based on cell-free RNA (cfRNA) expression data previously-obtained from a biological fluid sample (e.g., a blood sample) from the subject, the method comprising: obtaining artificial cfRNA expression data, the artificial cfRNA expression data comprising (i) a first plurality of artificial cfRNA expression profiles representing samples from subjects having the characteristic and (ii) a second plurality of artificial cfRNA expression profiles representing samples from subjects not having the characteristic; and training the machine learning model to predict the characteristic of the subject using the artificial cfRNA expression data, a particular artificial cfRNA expression profile of the first plurality of artificial cfRNA expression profiles having been generated by: generating a first healthy expression profile component by: receiving a first plurality of RNA expression profiles previously-obtained from biological samples from healthy subjects, the first plurality of RNA expression profiles including a respective RNA expression profile for each of one or more cell types and/or each of one or more types of cell-containing samples; and generating the first healthy expression profile component by combining the first plurality of RNA expression profiles; generating a first tumor expression profile component; and generating the particular artificial cfRNA expression profile by combining the first healthy expression profile component and the first tumor expression profile component.

Embodiments of any of the above aspects may have one or more of the following features.

In some embodiments, method of claim, training the machine learning model to predict the characteristic of the subject comprises training the machine learning model to predict whether the subject has cancer, such as e.g., breast cancer. In some embodiments, the first plurality of artificial cfRNA expression profiles represent samples from subjects having cancer such as, e.g., breast cancer. In some embodiments, the second plurality of artificial cfRNA expression profiles represent samples from subjects not having cancer, such as e.g., not having breast cancer (e.g., healthy subjects).

In some embodiments, a particular artificial cfRNA expression profile of the second plurality of artificial cfRNA expression profiles (e.g. one or more or each of the artificial cfRNA expression profiles of the second plurality of artificial cfRNA expression profiles) has been generated by: generating a second healthy expression profile component by: receiving a second plurality of RNA expression profiles previously-obtained from biological samples from healthy subjects, the second plurality of RNA expression profiles including a respective RNA expression profile for each of the one or more cell types and/or each of the one or more types of cell-containing samples; and generating the second healthy expression profile component by combining the second plurality of RNA expression profiles, wherein the particular artificial cfRNA expression profile corresponds to the generated second healthy expression profile component.

In some embodiments, training the machine learning model to predict the characteristic of the subject comprises training the machine learning model to predict whether the subject has liver metastasis In some embodiments, the first plurality of artificial cfRNA expression profiles represents samples from subjects having liver metastasis. In some embodiments, the second plurality of artificial cfRNA expression profiles represents samples from subjects not having liver metastasis (e.g., healthy subjects).

In some embodiments, the first plurality of RNA expression profiles include at least one RNA expression profile for liver tissue.

In some embodiments, a particular artificial cfRNA expression profile of the second plurality of artificial cfRNA expression profiles has been generated by: generating a second healthy expression profile component by: receiving a second plurality of RNA expression profiles previously-obtained from biological samples from healthy subjects, the second plurality of RNA expression profiles including a respective RNA expression profile for each of one or more cell types and/or each of one or more types of cell-containing samples excluding liver tissue; and generating the second healthy expression profile component by combining the second plurality of RNA expression profiles; generating a second tumor expression profile component; and generating the particular artificial cfRNA expression profile by combining the second healthy expression profile component and the second tumor expression profile component.

In some embodiments, training the machine learning model to predict the characteristic of the subject comprises training the machine learning model to predict a PD-1 status of the subject. In some embodiments, the first plurality of artificial cfRNA expression profiles represent samples from subjects that express PDCD1. In some embodiments, the second plurality of artificial cfRNA expression profiles represent samples from subjects that do not express PDCD1.

In some embodiments, the first tumor expression profile component has been generated using a first tumor expression profile from a tumor sample, the first tumor expression profile representing expression of PDCD1.

In some embodiments, a particular artificial cfRNA expression profile of the second plurality of artificial cfRNA expression profiles has been generated by: generating a second healthy expression profile component by: receiving a second plurality of RNA expression profiles previously-obtained from biological samples from healthy subjects, the second plurality of RNA expression profiles including a respective RNA expression profile for each of one or more cell types and/or each of one or more types of cell-containing samples; and generating the second healthy expression profile component by combining the second plurality of RNA expression profiles; generating a second tumor expression profile component, the second tumor expression profile component having been generated using a second tumor expression profile from a tumor sample that does not represent expression of PDCD1; and generating the particular artificial cfRNA expression profile by combining the second healthy expression profile component and the second tumor expression profile component.

In some embodiments, the first plurality of artificial cfRNA expression profiles and the second plurality of artificial cfRNA expression profiles each comprises at least 100 artificial cfRNA expression profiles, at least 250 artificial cfRNA expression profiles, at least 500 artificial cfRNA expression profiles, at least 1,000 artificial cfRNA expression profiles, at least 1,500 artificial cfRNA expression profiles, at least 2,000 artificial cfRNA expression profiles, at least 3,000 artificial cfRNA expression profiles, at least 4,000 artificial cfRNA expression profiles, or at least 5,000 cfRNA expression profiles.

In some embodiments, generating the healthy expression profile component by combining the plurality of RNA expression profiles comprises combining the plurality of RNA expression profiles and a cfRNA expression profile previously-obtained from a biological fluid sample from a healthy subject.

Some aspects provide for a method of generating artificial cell-free RNA (cfRNA) expression data used to train a machine learning model to predict a characteristic of a subject, the method comprising: using at least one computer hardware processor to perform: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile using the healthy expression profile component and the tumor expression profile component.

Some aspects provide for a method of generating artificial cell-free RNA (cfRNA) expression data used to train a machine learning model to predict a characteristic of a subject, the method comprising: using at least one computer hardware processor to perform: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile by combining the healthy expression profile component and the tumor expression profile component.

Embodiments according to the present aspects may have any of the features described herein in relation to steps of generating artificial cell-free RNA (cfRNA) expression data in any other aspect described herein. Further, embodiments of any other aspect described herein may have any of the features described in relation to the present aspect.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of generating artificial cell-free RNA (cfRNA) expression data used to train a machine learning model to predict a characteristic of a subject, the method comprising: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile using the healthy expression profile component and the tumor expression profile component.

Some aspects provide for at least one non-transitory computer-readable storage medium storing processor-executable instructions that, when executed by at least one computer hardware processor, causes the at least one computer hardware processor to perform a method of generating artificial cell-free RNA (cfRNA) expression data used to train a machine learning model to predict a characteristic of a subject, the method comprising: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: obtaining a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile using the healthy expression profile component and the tumor expression profile component.

Some aspects provide for a system, comprising: at least one computer hardware processor; and at least one non-transitory computer-readable storage medium storing processor-executable instructions that, when executed by the at least one computer hardware processor, causes the at least one computer hardware processor to perform a method of generating artificial cell-free RNA (cfRNA) expression data used to train a machine learning model to predict a characteristic of a subject, the method comprising: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile by combining the healthy expression profile component and the tumor expression profile component.

Some aspects provide for at least one non-transitory computer-readable storage medium storing processor-executable instructions that, when executed by at least one computer hardware processor, causes the at least one computer hardware processor to perform a method of generating artificial cell-free RNA (cfRNA) expression data used to train a machine learning model to predict a characteristic of a subject, the method comprising: generating the artificial cfRNA expression data by generating a plurality of artificial cfRNA expression profiles, wherein generating a particular artificial cfRNA expression profile of the plurality of artificial cfRNA expression profiles comprises: generating a healthy expression profile component by: receiving a plurality of RNA expression profiles previously-obtained from biological samples from healthy subjects, the plurality of RNA expression profiles including a respective RNA expression profile for each of one or more cell types and/or each of one or more types of cell-containing samples; and generating the healthy expression profile component by combining the plurality of RNA expression profiles; generating a tumor expression profile component; and generating the artificial cfRNA expression profile by combining the healthy expression profile component and the tumor expression profile component.

Embodiments of any of the above aspects (e.g. any of the methods of predicting a characteristic of a subject, methods of training a machine learning model and/or methods of generating artificial cfRNA expression data) may have one or more of the following features.

In some embodiments, generating the tumor expression profile component comprises: receiving a tumor expression profile previously-obtained from a tumor sample, the tumor expression profile comprising a plurality of counts for a respective plurality of genes, wherein the counts are counts of reads from RNA sequencing; determining, using the plurality of counts, a plurality of sampling probabilities including a respective sampling probability for each of the plurality of genes; sampling a plurality of reads from a multinomial distribution using at least some of the plurality of sampling probabilities, each of the plurality of reads corresponding to a gene of the plurality of genes; and generating the tumor expression profile component by summing, for each particular gene of the plurality of genes, a number of sampled reads corresponding to the particular gene. Thus, the tumor expression profile component may comprise a plurality of counts for a respective plurality of genes generated by sampling reads for the plurality of genes using respective sampling probabilities determined for each of the plurality of genes using a tumor expression profile previously-obtained from a tumor sample, the tumor expression profile comprising a plurality of measured counts for the respective plurality of genes.

In some embodiments, generating the tumor expression profile component comprises receiving a tumor expression profile previously-obtained from a tumor sample, the tumor expression profile comprising a plurality of counts for a respective plurality of genes, wherein the counts are counts of reads from RNA sequencing; determining, using the plurality of counts, a plurality of sampling probabilities including a respective sampling probability for each of the plurality of genes; sampling a plurality of reads using at least some of the plurality of sampling probabilities, each of the plurality of reads corresponding to a gene of the plurality of genes; and generating the tumor expression profile component by summing, for each particular gene of the plurality of genes, a number of sampled reads corresponding to the particular gene. Thus, the tumor expression profile component may comprise a plurality of counts for a respective plurality of genes generated by sampling reads for the plurality of genes using respective sampling probabilities determined for each of the plurality of genes using a tumor expression profile previously-obtained from a tumor sample, the tumor expression profile comprising a plurality of measured counts for the respective plurality of genes.

In some embodiments, the plurality of reads comprises a number of reads determined by sampling a value from a uniform distribution.

In some embodiment, combining the healthy expression profile component and the tumor expression profile component comprises determining a weighted combination of the healthy expression profile component and the tumor expression profile component, wherein the tumor expression profile component is weighted using the value sampled from the uniform distribution.

In some embodiments, generating the tumor expression profile component comprises generating the tumor expression profile component using a tumor expression profile previously-obtained from a tumor sample from a subject having breast cancer.

In some embodiments, the one or more cell types include macrophages, monocytes, granulocytes, fibroblasts, endothelium cells, and/or lymphocytes.

In some embodiments, the one or more types of cell-containing samples include peripheral blood mononuclear cell (PBMC) and/or whole blood.

In some embodiments, generating the healthy expression profile component further comprises determining a respective proportion for each of the plurality of RNA expression profiles by sampling the respective proportion from a Dirichlet distribution.

In some embodiments, combining the plurality of RNA expression profiles comprises determining a weighted combination of the plurality of RNA expression profiles. In some embodiments, RNA expression profiles of the plurality of RNA expression profiles are weighted using the respective proportion determined for each of the plurality of RNA expression profiles.

In some embodiments, generating the healthy expression profile component further comprises combining the plurality of RNA expression profiles and a cfRNA expression profile previously-obtained from a biological fluid sample from a healthy subject.

In some embodiments, combining the plurality of RNA expression profiles and the cfRNA expression profile comprises: combining the plurality of RNA expression profiles to obtain and initial healthy expression profile component; and determining a weighted combination of the initial healthy expression profile component and the cfRNA expression profile.

In some embodiments, generating the artificial cfRNA expression data comprises generating at least at least 100 artificial cfRNA expression profiles including the particular artificial cfRNA expression profile, at least 250 artificial cfRNA expression profiles including the particular artificial cfRNA expression profile, at least 500 artificial cfRNA expression profiles including the particular artificial cfRNA expression profile, at least 1,000 artificial cfRNA expression profiles including the particular artificial cfRNA expression profile, at least 1,500 artificial cfRNA expression profiles including the particular artificial cfRNA expression profile, at least 2,000 artificial cfRNA expression profiles including the particular artificial cfRNA expression profile, at least 2,500 artificial cfRNA expression profiles including the particular artificial cfRNA expression profile, at least 3,000 artificial cfRNA expression profiles including the particular artificial cfRNA expression profile, at least 4,000 artificial cfRNA expression profiles including the particular artificial cfRNA expression profile, at least 5,000 artificial cfRNA expression profiles including the particular artificial cfRNA expression profile, or at least 10,000 artificial cfRNA expression profiles including the particular artificial cfRNA expression profile.

Some embodiments further comprise training the machine learning model to predict the characteristic of the subject using the artificial cfRNA expression data.

In some embodiments, training the machine learning model to predict the characteristic of the subject comprises training the machine learning model to predict whether the subject has cancer.

In some embodiments, training the machine learning model to predict the characteristic of the subject comprises training the machine learning model to predict whether the subject has liver metastasis.

In some embodiments, training the machine learning model to predict the characteristic of the subject comprises training the machine learning model to predict a fraction of malignant B cells relative to a total number of B cells for the subject.

In some embodiments, training the machine learning model to predict the characteristic of the subject comprises training the machine learning model to predict a PD-1 status for the subject.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a diagram of an illustrative technique for using a machine learning model, trained using artificial cell-free RNA (cfRNA) expression data to predict a characteristic of a subject, according to some embodiments of the technology described herein.

FIG. 1B is a diagram of an illustrative technique for training a machine learning model to predict a characteristic of a subject using artificial cfRNA expression data, according to some embodiments of the technology described herein.

FIG. 1C, FIG. 1D, and FIG. 1E are diagrams of an illustrative technique for generating artificial cfRNA expression data, according to some embodiments of the technology described herein.

FIG. 1F is a block diagram of an example system for using a machine learning model, trained using artificial cfRNA expression data, to predict a characteristic of a subject, according to some embodiments of the technology described herein.

FIG. 2A is a flowchart of an illustrative process for using a machine learning model, trained using artificial cfRNA expression data, to predict a characteristic of a subject, according to some embodiments of the technology described herein.

FIG. 2B is a flowchart of an illustrative process for training a machine learning model to predict a characteristic of a subject using artificial cfRNA expression data, according to some embodiments of the technology described herein.

FIG. 2C is a flowchart of an illustrative process for generating artificial cfRNA expression data, according to some embodiments of the technology described herein.

FIG. 3A, FIG. 3B, and FIG. 3C show that a machine learning model trained using artificial cfRNA expression data, according to some embodiments of the technology described herein, accurately distinguishes between subjects having liver metastasis and subjects not having liver metastasis.

FIG. 4A, FIG. 4B, and FIG. 4C show that a machine learning model trained using artificial cfRNA expression data, according to some embodiments of the technology described herein, accurately distinguishes between subjects having breast cancer and subjects not having breast cancer.

FIG. 5A shows correlations between cfRNA-seq and peripheral blood mononuclear cells (PBMC) RNA-seq-based deconvolution, according to some embodiments of the technology described herein.

FIG. 5B and FIG. 5C show the changes in immune cell population before and after treatment, according to some embodiments of the technology described herein.

FIG. 5D show that the dominant B-cell receptor (BCR) clonotypes were concordant between cfRNA-seq and PBMC RNA-seq, according to some embodiments of the technology described herein.

FIG. 5E and FIG. 5F shows that a machine learning model trained using artificial cfRNA expression data, according to some embodiments of the technology described herein, accurately predicts malignant B cell fraction relative to conventional techniques for determining malignant B cell fraction.

FIG. 5G shows that tumor-derived mutations were successfully called from cfRNA transcriptome, according to some embodiments of the technology described herein.

FIG. 5H shows that the malignant B cell fraction, predicted according to embodiments of the technology described herein, can be used to accurately determine whether a subject has chronic lymphocytic leukemia (CLL).

FIG. 5I and FIG. 5J show results of validating the limit of detection and limit of quantification used for determining whether a subject has CLL, according to embodiments of the technology described herein.

FIG. 6A and FIG. 6B shows that a machine learning model trained using artificial cfRNA expression data, according to some embodiments of the technology described herein, accurately distinguishes between subjects having tumor cells that express PDCD1 and subjects having tumor cells that do not express PDCD1.

FIG. 7A shows the cohorts used for model testing, according to some embodiments of the technology described herein.

FIG. 7B shows that robust cfRNA extraction and sequencing protocols can be used to obtain reproducible profiling of cfRNA transcriptomes, according to some embodiments of the technology described herein.

FIG. 7C shows that cfRNA transcriptomes contain tumor-derived transcripts, according to some embodiments of the technology described herein.

FIG. 7D shows that tumor-related signatures are enriched in cfRNA transcriptomes from sarcoma and carcinoma patients compared to healthy donors, according to some embodiments of the technology described herein.

FIG. 7E shows that machine learning models trained using artificial cfRNA expression data, according to some embodiments of the technology described herein, can be used to accurately predict whether a subject has breast cancer, the tumor microenvironment (TME) fibrosis status of a subject, the PD-1 status of a subject, and whether a subject has liver metastasis.

FIG. 8 shows results indicating high reproducibility of cfRNA sequencing results and results of processing artificial cfRNA expression data using machine learning models trained to identify primary tumor signals and detect PD1 expression, according to some embodiments of the technology described herein.

FIG. 9 is a schematic diagram of an illustrative computing device with which aspects described herein may be implemented.

FIG. 10 shows that a machine learning model trained using artificial cfRNA expression data, according to some embodiments of the technology described herein, accurately distinguishes between subjects having basal breast cancer and subjects not having basal breast cancer.

### DETAILED DESCRIPTION

Cell-free RNA (cfRNA) exists in biological fluids independent of cells. Thus, cfRNA can be detected and measured in a minimally invasive manner by analyzing a fluid sample (e.g., a blood sample) obtained from a subject. For example, cfRNA may be detected and measured by sequencing (e.g., RNA sequencing (RNA-seq)) a fluid biological sample, resulting in expression levels for a plurality of genes. The gene expression levels can be used as biomarkers to predict a characteristic of a subject such as whether the subject has a particular characteristic (e.g., cancer, metastasis, etc.), the subject's response to a particular therapy, whether the subject has minimal residual disease (MRD), and/or whether the subject is experiencing or is likely to experience an adverse event, among other characteristics. Methods of the present disclosure are applicable in the context of predicting any such characteristic.

Conventional techniques for predicting a characteristic of a subject using cfRNA expression data involve processing measured cfRNA expression data using a machine learning model trained to predict the particular characteristic of the subject. However, the inventors have recognized that a problem associated with the conventional techniques is that there is insufficient training data available to train a machine learning model to predict a characteristic of a subject. Therefore, conventional techniques involve acquiring training data by physically sequencing blood samples from subjects from each class (e.g., subjects having the particular characteristic and subjects not having the particular characteristic). This is inefficient and time consuming because it involves identifying individuals from each class, obtaining and preparing blood samples from each individual, and sequencing the blood samples. Furthermore, the lack of established practices associated with obtaining and preparing blood samples leads to process-dependent variabilities among the resulting sequencing data. The inefficiencies of collecting the data and the variabilities among the collected data impose constraints on scalability, making it challenging to acquire enough training data to train even one machine learning model to predict a characteristic of a subject, never mind to train multiple machine learning models to predict multiple characteristics of a subject.

Accordingly, the inventors have developed machine learning techniques that address the above-described challenges associated with the conventional machine learning techniques for predicting a characteristic of a subject using cfRNA expression data. The techniques developed by the inventors involve predicting a characteristic of a subject based on cell-free RNA (cfRNA) expression data previously-obtained from a biological fluid sample (e.g., a bloodsample). The use of artificial cfRNA expression data enables the generation of large amounts of training data without requiring that physical biological fluid samples (e.g., blood samples) be obtained from individuals from each class. Furthermore, artificial cfRNA expression data can be generated without preparing and/or sequencing a vast number of physical samples, thereby eliminating the process-dependent variabilities that impose constraints on scalability. Accordingly, the techniques developed by the inventors can be used to more efficiently and reliably generate training data at a large scale as compared to the conventional machine learning techniques for predicting a characteristic of a subject.

In particular, in some embodiments, the techniques developed by the inventors involve: obtaining cfRNA expression data previously-obtained from a biological fluid sample (e.g, a blood sample) from a subject; and processing the cfRNA expression data using a machine learning model trained to process cfRNA expression data from a subject and produce an output indicative of the characteristic of the subject. In some embodiments, the trained machine learning model was trained using artificial cfRNA expression data comprising artificial cfRNA expression profiles. In some embodiments, an artificial RNA expression profile is generated by: (a) generating a healthy expression profile component; (b) generating a tumor expression profile component; and (c) generating the artificial cfRNA expression profile by combining (e.g., determining a weighted sum of) the healthy expression profile component and the tumor expression profile component. In some embodiments, generating the healthy RNA expression component involves: receiving a plurality of RNA expression profiles previously-obtained from biological samples from healthy subjects, the plurality of RNA expression profiles including a respective RNA expression profile for each of one or more cell types and/or each of one or more types of cell-containing samples (e.g., tissue types); and generating the healthy expression profile component by combining (e.g., determining a weighted sum of) the plurality of RNA expression profiles. By generating artificial expression profiles that account for expression associated with tumor cells and different types of healthy cells and/or tissues, the techniques developed by the inventors accurately account for the diverse types of expression profiles that are typically present in a biological fluid sample such as a blood sample.

Following below are descriptions of various concepts related to, and embodiments of, using a machine learning model, trained using artificial cfRNA expression data, to predict a characteristic of a subject. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

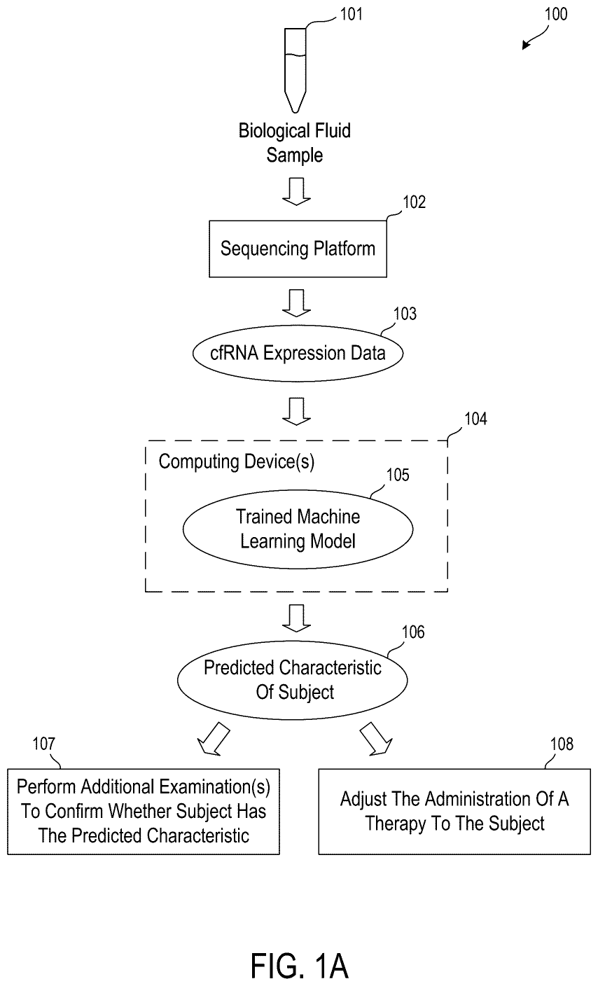

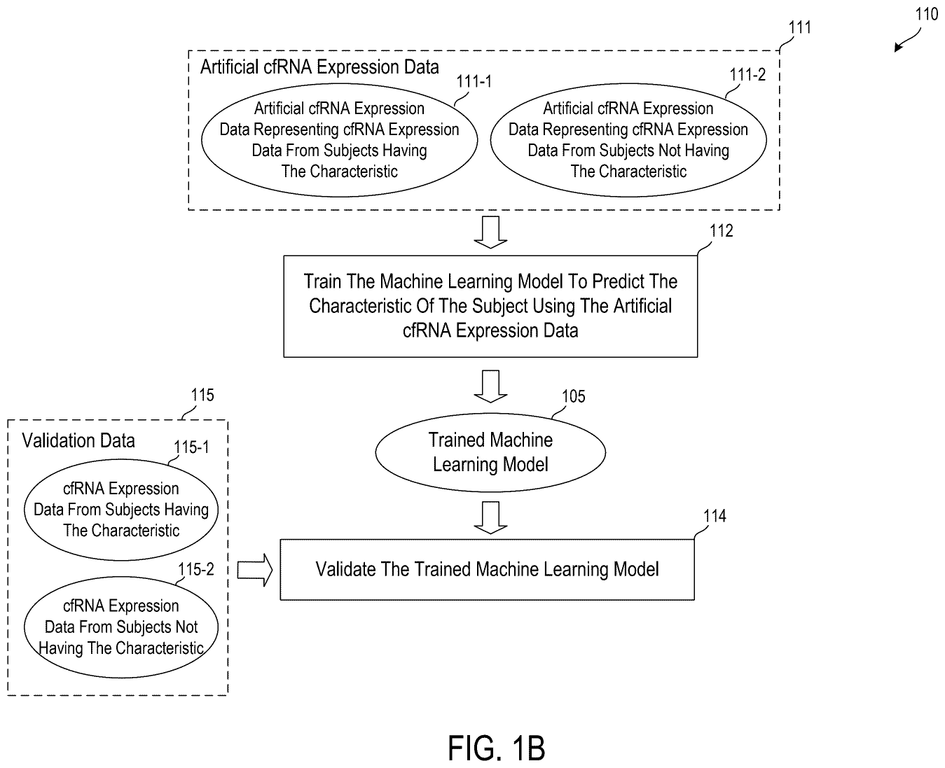

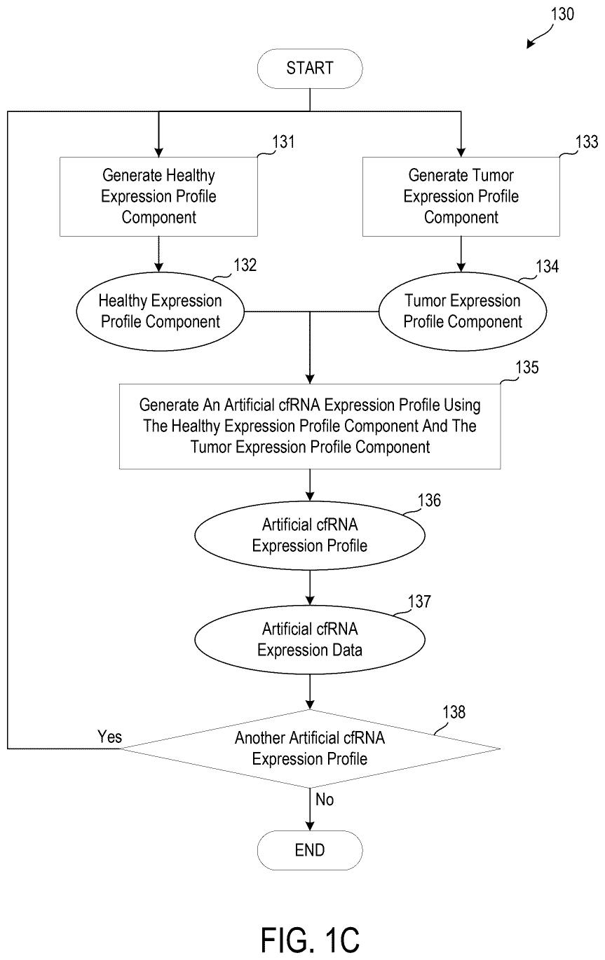

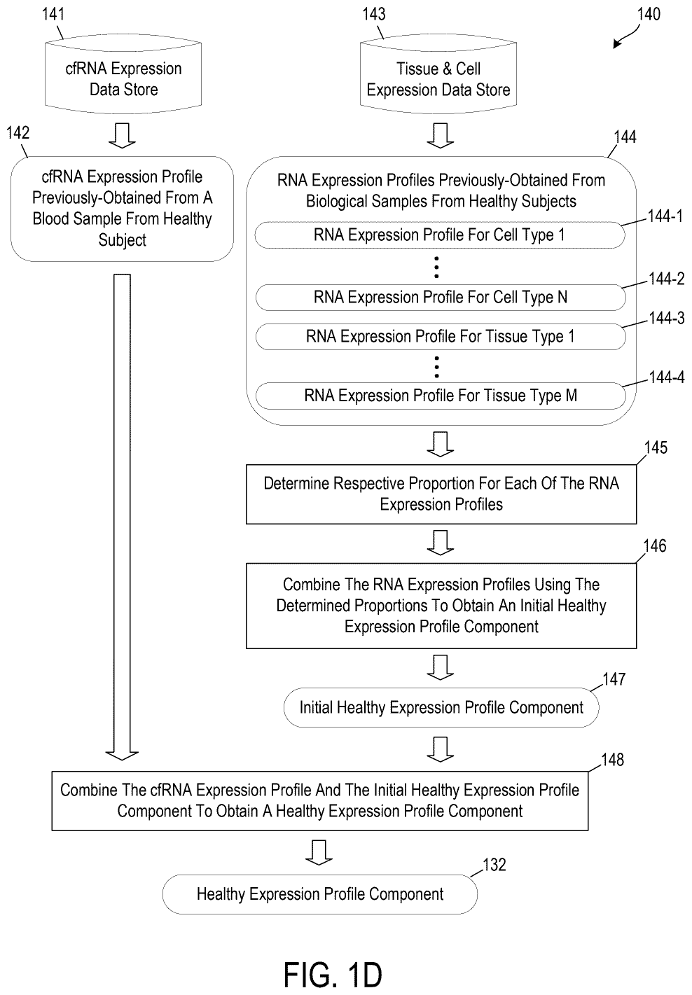

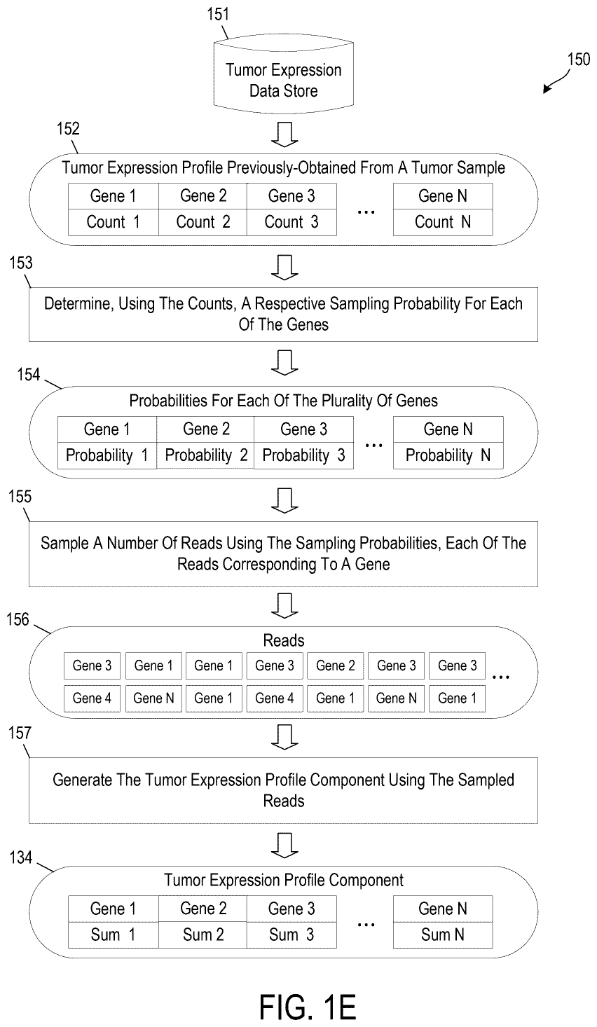

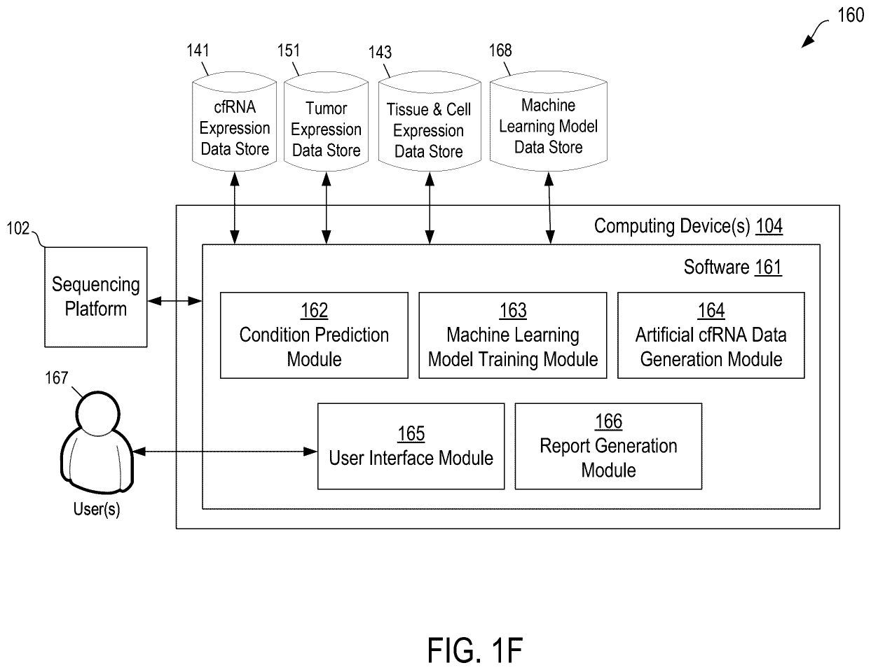

FIG. 1A is a diagram depicting an illustrative technique 100 for using a machine learning model 105, trained using artificial cell-free RNA (cfRNA) expression data, to predict a characteristic of a subject, according to some embodiments of the technology described herein. Illustrative technique 100 includes processing cfRNA expression data 103 using a trained machine learning model 105 on computing device(s) 104 to predict a characteristic 106 of a subject. In some embodiments, the cfRNA expression data 103 is obtained by sequencing a biological fluid sample 101 from the subject using sequencing platform 102. In some embodiments, the predicted characteristic 106 of the subject informs one or more downstream acts (e.g., act 107 and/or act 108) relating to examining and/or treating the subject.

In some embodiments, cfRNA expression data 103 is obtained by sequencing a biological fluid sample 101. A biological fluid sample, in some embodiments, refers to a sample comprising cells, e.g., cells from a biological fluid sample. In some embodiments, the biological fluid sample comprises non-cancerous cells. In some embodiments, the biological fluid sample comprises precancerous cells. In some embodiments, the biological fluid sample comprises cancerous cells. In some embodiments, the biological fluid sample is a blood sample. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. A sample of blood may be a sample of whole blood or a sample of fractionated blood. In some embodiments, the sample of blood comprises buffy coat. In some embodiments, the sample of blood comprises serum. In some embodiments, the sample of blood comprises plasma. In some embodiments, the sample of blood comprises a blood clot. In some embodiments, the biological fluid sample is saliva. In some embodiments, the biological fluid sample is urine. Aspects of biological fluid samples and techniques for obtaining and/or preparing biological fluid samples are described herein including at least in the section “Biological Samples.”

In some embodiments, the sequencing platform 102 is a next generation sequencing platform (e.g., Illumina™, Roche™, Ion Torrent™, etc.), or any high-throughput or massively parallel sequencing platform. In some embodiments, the sequencing platform 102 is configured to perform RNA sequencing (RNA-seq). In some embodiments, the sequencing platform 102 is configured to perform single-cell RNA-seq (scRNA-seq). In some embodiments, the RNA-seq is whole RNA-seq and/or mRNA-seq. Examples techniques for obtaining expression data, including cfRNA expression data, are described herein including at least in the section entitled “Expression Data.”

In some embodiments, the cfRNA expression data 103 includes at least one expression profile. In some embodiments, an expression profile indicates expression levels for a plurality of genes. The number of genes which may be examined may be up to and inclusive of all the genes of the subject. In some embodiments, expression levels may be examined for all of the genes of a subject. For example, the number of genes may include at least 2, at least 5, at least 10, at least 25, at least 50, at least 75, at least 100, at least 150, at least 200, at least 500, at least 1,000, at least 2,000, at least 3,000, at least 4,000, at least 5,000, at least 6,000, at least 7,000, at least 8,000, at least 9,000, at least 10,000, at least 15,000, at least 20,000, at least 25,000, at least 30,000, at least 40,000, at least 50,000, or at least any other suitable number of genes, as aspects of the technology described herein are not limited in this respect. In some embodiments, the number of genes may include at most 5 at most 10, at most 25, at most 50, at most 75, at most 100, at most 150, at most 200, at most 250, at most 300, at most 350, at most 400, at most 450, at most 500, at most 1,000, at most 2,000, at most 3,000, at most 4,000, at most 5,000, at most 6,000, at most 7,000, at most 8,000, at most 9,000, at most 10,000, at most 15,000, at most 20,000, at most 25,000, at most 30,000, at most 40,000, at most 50,000, or any other suitable number of genes, as aspects of the technology described herein are not limited in this respect. It should be appreciated that any of the above-listed upper bounds may be coupled with any of the above-listed lower bounds.

In some embodiments, an expression profile included in the cfRNA expression data 103 includes the genes listed in Table 2, Table 5, Table 10, and/or Table 13. The expression profile may include at least some (e.g., all) of the genes listed in Table 2. For example, when the machine learning model 105 is trained to predict whether a subject has liver metastasis, the expression profile may include at least some (e.g., all) of the genes listed in Table 2. The expression profile may include at least some (e.g., all) of the genes listed in Table 5. For example, when the machine learning model 105 is trained to predict whether a subject has breast cancer, the expression profile may include at least some (e.g., all) of the genes listed in Table 5. The expression profile may include at least some (e.g., all) of the genes listed in Table 10. For example, when the machine learning model 105 is trained to predict whether a subject has tumor cells that express PDCD1, the expression profile may include at least some (e.g., all) of the genes listed in Table 10. The expression profile may include at least some (e.g., all) of the genes listed in Table 13. For example, when the machine learning model 105 is trained to predict whether a subject has basal breast cancer, the expression profile may include at least some (e.g., all) of the genes listed in Table 13.

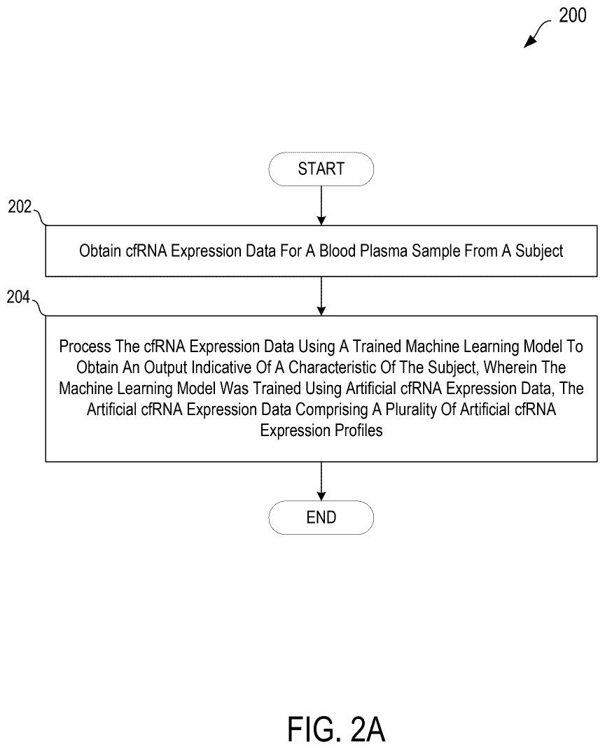

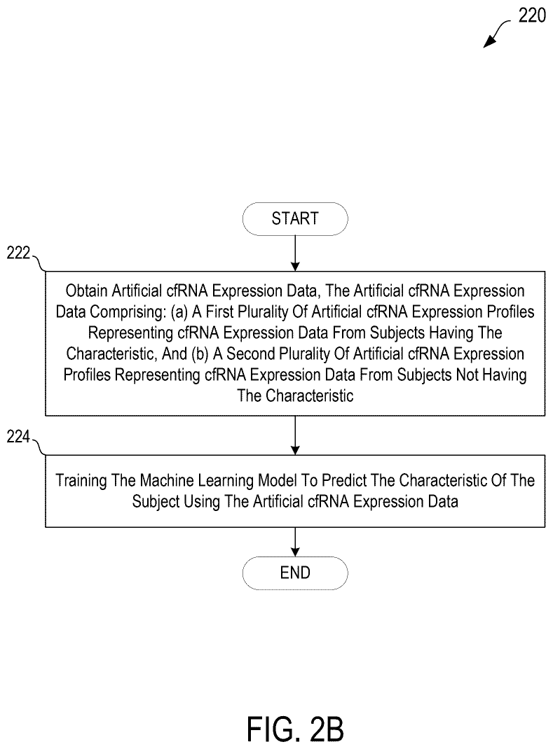

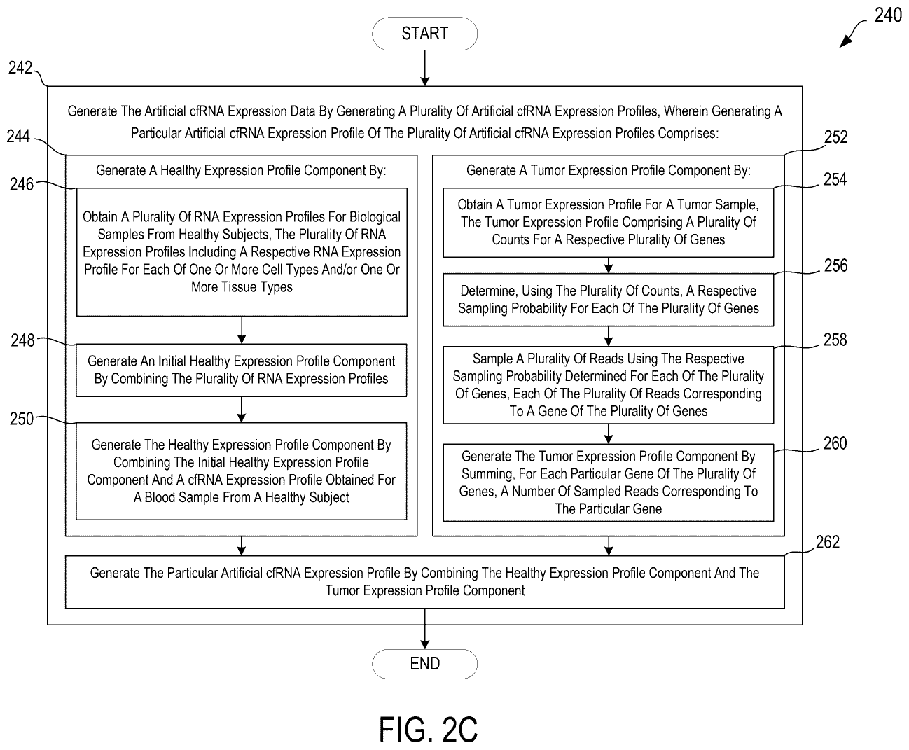

In some embodiments, software (e.g., software 161 shown in FIG. 1F) on computing device(s) 104 is configured to process the cfRNA expression data 103 to predict the characteristic 106 of the subject. In some embodiments, this includes processing the cfRNA expression data 103 using a machine learning model 105 trained to predict the characteristic 106 of the subject. In some embodiments, the machine learning model 105 was trained using artificial cfRNA expression data. Example techniques for training a machine learning model to predict a characteristic of a subject are described herein including at least with respect to FIG. 1B and FIG. 2B. Example techniques for generating artificial cfRNA expression data are described herein including at least with respect to FIG. 1C, FIG. 1D, FIG. 1E, and FIG. 2C.

In some embodiments, the machine learning model 105 is trained to predict a characteristic 106 of the subject. In some embodiments, the output of the machine learning model is a prediction of whether or not the subject has the characteristic. For example, output may be a binary indication of whether or not the subject has the characteristic. For example, the output may indicate whether or not the subject has cancer (e.g., breast cancer, basal cancer, etc.). The output may indicate whether or not the subject has tumor cells that express PDCD1 (e.g., PDCD1+). In some embodiments, the output is a likelihood (e.g., a probability) that the subject has the characteristic. In some embodiments, the output is a predicted value of a characteristic. For example, the machine learning model 105 may be trained to predict the fraction of malignant B cells relative to a total number of B cells. However, it should be appreciated that the output may include any other suitable output, as aspects of the technology described herein are not limited to any particular output format.

In some embodiments, the machine learning model is a machine learning model trained to predict whether the subject has breast cancer. The machine learning model may be trained to process cfRNA expression data for at least some (e.g., all) of the genes listed in Table 5. The machine learning model may be a trained LightGBM decision tree boosting machine learning model to predict whether the subject has breast cancer. LightGBM is described by Ke, Guolin, et al. (“Lightgbm: A highly efficient gradient boosting decision tree.” Advances in neural information processing systems 30 (2017).), which is incorporated by reference herein in its entirety. For example, the LightGBM decision tree boosting machine learning model may be the LightGBM decision tree boosting machine learning model described herein with respect to Example 2. For example, the LightGBM decision tree boosting machine learning model may have the parameters listed in Table 6.

In some embodiments, the machine learning model is a machine learning model trained to predict whether the subject as liver metastasis. The machine learning model may be trained to process cfRNA expression data for at least some (e.g., all) of the genes listed in Table 2. The machine learning model may be a trained LightGBM decision tree boosting machine learning model to predict whether the subject has liver metastasis. For example, the LightGBM decision tree boosting machine learning model may be the LightGBM decision tree boosting machine learning model described herein with respect to Example 1. For example, the LightGBM decision tree boosting machine learning model may have the parameters listed in Table 3.

In some embodiments, the machine learning model is a machine learning model trained to predict a PD-1 status of the subject (e.g., whether the subject expresses PDCD1 (PDCD1+) or does not express PDCD1 (PDCD1−). The machine learning model may be trained to process cfRNA expression data for at least some (e.g., all) of the genes listed in Table 10. The machine learning model may be a trained LightGBM decision tree boosting machine learning model to predict whether the subject's tumor cells express PDCD1. For example, the LightGBM decision tree boosting machine learning model may be the LightGBM decision tree boosting machine learning model described herein with respect to Example 4. For example, the LightGBM decision tree boosting machine learning model may have the parameters listed in Table 11.

In some embodiments, the machine learning model is a machine learning model trained to predict a malignant B cell fraction for the subject. The machine learning model may be a trained LightGBM decision tree boosting machine learning model to predict the malignant B cell fraction for the subject. For example, the LightGBM decision tree boosting machine learning model may be the LightGBM decision tree boosting machine learning model described herein with respect to Example 3. For example, the LightGBM decision tree boosting machine learning model may have the parameters listed in Table 8.

In some embodiments, the machine learning model is a machine learning model trained to predict whether the subject has basal breast cancer. The machine learning model may be trained to process cfRNA expression data for at least some (e.g., all) of the genes listed in Table 13. The machine learning model may be a trained LightGBM decision tree boosting machine learning model to predict whether the subject has basal breast cancer. For example, the LightGBM decision tree boosting machine learning model may be the LightGBM decision tree boosting machine learning model described herein with respect to Example 5. For example, the LightGBM decision tree boosting machine learning model may have the parameters listed in Table 14.

In some embodiments, the machine learning model is a decision tree model, a gradient boosted decision tree model, a linear regression model, a non-linear regression model, a support vector machine, a Gaussian mixture model, a random forest model, a neural network model, or any other suitable type of machine learning model, as aspects of the technology described herein are not limited in this respect. Aspects of machine learning models are described herein including at least in the section entitled “Machine Learning.” In some embodiments, the machine learning model is a classifier. In some embodiments, the machine learning model is trained to classify input cfRNA expression data between a first class associated with subjects that have a characteristic, and a second class associated with subjects that do not have the characteristic. Such machine learning models may produce as output an indication of whether the subject from whom the cfRNA expression data has been obtained has the characteristic. The indication may be e.g. a probability that the subject has the characteristic (i.e., a probability that the subject is classified in the first class).

In some embodiments, the predicted characteristic includes an indication of whether the subject has a disease and/or condition. The disease may be cancer. For example, the predicted characteristic may include a prediction of whether the subject has breast cancer, luminal A breast cancer, luminal B breast cancer, basal breast cancer, lung cancer, liver metastasis, bone metastasis, and/or any other suitable disease or condition, as aspects of the technology described herein are not limited in this respect. In some embodiments, the predicted characteristic includes a prediction of whether the subject has tumor cells that express PDCD1 (e.g., PDCD1+). In some embodiments, the predicted characteristic includes a prediction of a malignant B cell fraction (e.g., fraction of malignant B cells relative to total B cells) of the subject. In some embodiments, the predicted characteristic includes a prediction of the subject's response to administration of a therapy. For example, the predicted characteristic may include a prediction of whether a subject will respond to an immune checkpoint inhibitor (ICI) therapy. In some embodiments, the predicted characteristic includes a prediction of the subject's survival prognosis. For example, the predicted characteristic may include a prediction of whether the subject belongs to a first class associated with a first prognosis (e.g., poor prognosis) or a second class associated with a second prognosis that is better than the first prognosis (e.g., good prognosis).

In some embodiments, the predicted characteristic 106 is used to inform one or more subsequent acts (e.g., act 107 and/or act 108). For example, the predicted characteristic 106 may be used to generate a report that includes a recommendation to perform one or more subsequent acts (e.g., act 107 and/or act 108). In some embodiments, at act 107, one or more additional examinations (e.g. one or more diagnostic tests) are performed to confirm whether the subject has the predicted characteristic 106. For example, if the subject is predicted to have breast cancer (e.g., basal breast cancer), act 107 may include performing a biopsy and/or mammography. Additionally, or alternatively, if the subject is predicted to have liver metastasis, act 107 may include performing an ultrasound and/or a biopsy. In some embodiments, at act 107, the predicted characteristic 106 is used to inform a diagnosis of the subject. For example, the malignant B cell fraction predicted for a subject may be used to determine whether the subject has chronic lymphocytic leukemia (CLL). In some embodiments, at act 108, therapy administration is adjusted. Thus, the method may comprise recommending whether and/or how to adjust therapy for the subject. For example, if the subject is predicted to have a positive response to a particular therapy, then the therapy may be administered or increased. Alternatively, if the subject is predicted to have no response or an adverse response to the particular therapy, then administration of the therapy may be stopped or decreased. Additionally, or alternatively, if the subject is predicted to no longer have a particular disease or condition, then therapy administration may be stopped or decreased.

FIG. 1B is a diagram of an illustrative technique 110 for training a machine learning model to predict a characteristic of a subject using artificial cfRNA expression data, according to some embodiments of the technology described herein. In some embodiments, the illustrative technique 110 includes (a) obtaining artificial cfRNA expression data 111, (b) at act 112, training the machine learning model to predict the characteristic of the subject using the artificial cfRNA expression data 111, and (c) in some embodiments, at (optional) act 114, validating the trained machine learning model 105 using validation data 115.

In some embodiments, the artificial cfRNA expression data 111 includes two subsets of artificial cfRNA expression data. The first subset 111-1 includes artificial cfRNA expression data representing cfRNA expression data from subjects having the particular characteristic. The second subset 111-2 includes artificial cfRNA expression data representing cfRNA expression data from subjects not having the particular characteristic. For example, if the machine learning model is trained to predict whether the subject has breast cancer, then the first subset 111-1 may include artificial cfRNA expression data representing cfRNA expression data from subjects having breast cancer, and the second subset 111-2 may include artificial cfRNA expression data from subjects not having breast cancer.

In some embodiments, each of the subsets 111-1 and 111-2 includes a plurality of artificial cfRNA expression profiles. For example, the plurality of artificial cfRNA expression profiles may include at least 400, at least 500, at least 600, at least 700, at least 800, at least 900, at least 1,000, at least 1,250, at least 1,500, at least 1,750, at least 2,000, at least 2,250, at least 2,500, at least 2,750, at least 3,000, at least 3,500, at least 4,000, at least 4,500, at least 5,000, at least 5,500, at least 6,000, at least 7,000, at least 8,000, at least 9,000, at least 10,000, at least 15,000, at least 20,000, at least 25,000, at least 50,000, or at least any other suitable number of artificial cfRNA expression profiles. Additionally, or alternatively, the plurality of artificial cfRNA expression profiles may include at most 500, at most 600, at most 700, at most 800, at most 900, at most 1,000, at most 1,250, at most 1,500, at most 1,750, at most 2,000, at most 2,250, at most 2,500, at most 2,750, at most 3,000, at most 3,500, at most 4,000, at most 4,500, at most 5,000, at most 5,500, at most 6,000, at most 7,000, at most 8,000, at most 9,000, at most 10,000, at most 15,000, at most 20,000, at most 25,000, at most 50,000, at most 100,000, at most 200,000, or at most any other suitable number of artificial cfRNA expression profiles. It should be appreciated that any of the above-listed upper bounds may be coupled with any of the above-listed lower bounds.

In some embodiments, an artificial cfRNA expression profile includes an expression level (e.g. a count of reads) for each of a plurality of genes. The number of genes in an expression profile may be up to and inclusive of all the genes expected to be present in the genome of the subject. Thus, the artificial cfRNA expression profile may be a whole transcriptome cfRNA expression profile. The artificial cfRNA expression profile may be generated using measured RNA expression profiles (e.g. a plurality of RNA expression profiles previously-obtained from biological samples from healthy subjects, one or more cfRNA expression profiles previously obtained from blood samples from healthy subjects, and/or one or more tumor expression profiles previously obtained from tumor samples). Thus, the artificial cfRNA expression profile may include an expression level for each of a plurality of genes represented in the measured RNA expression profiles. As the skilled person understands, while the measured RNA expression profiles may have been obtained using assays designed to probe a whole transcriptome, the measured RNA expression profiles may not contain a measurement for every single gene in the transcriptome. In some embodiments, expression levels may be examined for all of the genes of a subject. For example, the number of genes may include at least 2, at least 5, at least 10, at least 25, at least 50, at least 75, at least 100, at least 150, at least 200, at least 500, at least 1,000, at least 2,000, at least 3,000, at least 4,000, at least 5,000, at least 6,000, at least 7,000, at least 8,000, at least 9,000, at least 10,000, at least 15,000, at least 20,000, at least 25,000, at least 30,000, at least 40,000, at least 50,000, or at least any other suitable number of genes, as aspects of the technology described herein are not limited in this respect. In some embodiments, the number of genes may include at most 5 at most 10, at most 25, at most 50, at most 75, at most 100, at most 150, at most 200, at most 250, at most 300, at most 350, at most 400, at most 450, at most 500, at most 1,000, at most 2,000, at most 3,000, at most 4,000, at most 5,000, at most 6,000, at most 7,000, at most 8,000, at most 9,000, at most 10,000, at most 15,000, at most 20,000, at most 25,000, at most 30,000, at most 40,000, at most 50,000, or any other suitable number of genes, as aspects of the technology described herein are not limited in this respect. It should be appreciated that any of the above-listed upper bounds may be coupled with any of the above-listed lower bounds.

In some embodiments, the artificial cfRNA expression data 111 is used to train the machine learning model to predict the characteristic of the subject. In some embodiments, the machine learning model is trained using any suitable training technique(s), including supervised techniques, as aspects of the technology described herein are not limited in this respect. As one example, in the supervised training context, an artificial cfRNA expression profile included in the artificial cfRNA expression data 111 may be provided as input to the machine learning model, which may output an indication of the predicted characteristic. Differences between the predicted characteristic and a known characteristic associated with the artificial cfRNA expression profile may be used to determine and update the parameter values of the machine learning model. For example, an artificial cfRNA expression profile from the first subset 111-1 may be provided as input to the machine learning model. If the machine learning model outputs a prediction that the subject has the particular characteristic, then the prediction is correct and is confirmed based on the known characteristic associated with the artificial cfRNA expression profile. If the machine learning model outputs a prediction that the subject does not have the particular characteristic, then the discrepancy between the predicted and known characteristic is used to determine and/or update the parameters of the machine learning model.

In some embodiments, after the machine learning model has been trained, the trained machine learning model 105 may be validated at act 114. In some embodiments, the machine learning model is validated using validation data 115. The validation data 115 may include a first subset 115-1 and a second subset 115-2 of validation data. In some embodiments, the first subset 115-1 includes cfRNA expression data obtained from blood samples from subjects having the particular characteristic, and the second subset 115-2 includes cfRNA expression data obtained from blood samples from subjects not having the particular characteristic.

In some embodiments, validating the trained machine learning model 105 includes evaluating the performance of the trained machine learning model. For example, validating the trained machine learning model 105 may include determining metrics that evaluate the ability of the machine learning model to distinguish between different characteristics (e.g., subjects having the characteristic versus subjects not having the characteristic) such as p-value, area under the receiving operating characteristic curve (AUC ROC), precision-recall AUC (PR AUC), and any other suitable performance metrics, as aspects of the technology described herein are not limited in this respect.

FIG. 1C is a diagram of an illustrative technique 130 for generating artificial cfRNA expression data 137, according to some embodiments of the technology described herein. In some embodiments, generating the artificial cfRNA expression data 137 includes generating a plurality of artificial cfRNA expression profiles including artificial cfRNA expression profile 136.

In some embodiments, generating an artificial cfRNA expression profile 136 includes (a) at act 131, generating a healthy expression profile component 132, (b) at act 133, generating a tumor expression profile component 134, and (c) at act 135, generating the artificial cfRNA expression profile 136 using the healthy expression profile component 132 and the tumor expression profile component 134. The generated artificial cfRNA expression profile 136 is then included in the artificial cfRNA expression data 137.

In some embodiments, the healthy expression profile component 132 represents cfRNA expression data resulting from cfRNA that has been released by healthy cells. An example implementation of act 131, for generating the healthy expression profile component 132, is described herein including at least with respect to FIG. 1D and FIG. 2C.

In some embodiments, the tumor expression profile component 134 represents cfRNA expression data resulting from cfRNA that has been released by tumor cells. An example implementation of act 133, for generating the tumor expression profile component 134, is described herein including at least with respect to FIG. 1E and FIG. 2C.

At act 135, the artificial cfRNA expression profile is generated using the healthy expression profile component 132 and the tumor expression profile component 134. In some embodiments, generating the artificial cfRNA expression profile (E) includes combining the healthy expression profile component 132 (E_{Healthy}) and the tumor expression profile component 134 (E_{Tumor}). For example, this may include determining a weighted sum of the healthy and tumor expression profile components, as shown in Equation 1:

$$E = γ {E}_{Tumor} + ( 1 - γ ) {E}_{Healthy} \qquad ( Equation 1 )$$

The proportion γ in Equation 1 may be determined using any suitable techniques, as aspects of the technology described herein are not limited in this respect. For example, the proportion γ may be determined by sampling from a uniform distribution. The lower bound of the uniform distribution may range between 10⁻⁸ and 10⁻⁴. For example, the lower bound may be 10⁻⁸, 10⁻⁷, 10⁻⁶, 10⁻⁵, or 10⁻⁴. The upper bound of the uniform distribution may range between 10⁻⁴ and 10⁻¹. For example, the upper bound may be 10⁻⁴, 10⁻³, 10⁻², or 10⁻¹. Thus, the uniform distribution may have a lower bound of 10⁻⁶ and an upper bound of 10⁻². For example, the proportion γ may be sampled from the uniform distribution: γ˜ Uniform (10⁻⁶, 10⁻²).

It should be appreciated that, in some embodiments, the tumor expression profile component 134 may be excluded, and the artificial cfRNA expression profile 136 may be generated using only the healthy expression profile component 132. In other words, the proportion γ in Equation 1, may be set to 0. For example, with reference to FIG. 1B, when training a machine learning model to predict whether the subject has cancer or does not have cancer, the second subset of artificial cfRNA expression data 111-2 (e.g., representing cfRNA expression data from subjects that do not have the characteristic) may include artificial cfRNA expression profiles that were generated using only healthy RNA expression profile components and not the tumor expression profile components. By contrast, the first subset of artificial cfRNA expression data 111-1 (e.g., representing cfRNA expression data from subjects having the characteristic) may include artificial cfRNA expression profiles generated using both healthy and tumor expression profile components.

At act 138, technique 130 includes determining whether to generate another artificial cfRNA expression profile for inclusion in the artificial cfRNA expression data 137. Any suitable number of artificial cfRNA expression profiles may be generated, as aspects of the technology described herein are not limited in this respect. For example, with reference to FIG. 1B, a first number of artificial cfRNA expression profiles may be generated for the first subset 111-1 of artificial cfRNA expression data and a second number of artificial cfRNA expression profiles may be generated for the second subset 111-2 of artificial cfRNA expression data.

FIG. 1D is a diagram of an illustrative technique 140 for generating a healthy expression profile component 132 used for generating an artificial cfRNA expression profile, according to some embodiments of the technology described herein. In some embodiments, the healthy expression profile component 132 is generated using (a) a cfRNA expression profile 142 previously obtained from a blood sample from a healthy subject, and (b) RNA expression profiles previously obtained from biological samples from healthy subjects.

In some embodiments, the cfRNA expression profile 142 is obtained from a cfRNA expression data store 141, from a sequencing platform (e.g., sequencing platform 102), by sequencing the blood sample obtained from the healthy subject, or in any other suitable way, as aspects of the technology described herein are not limited in this respect.

As described herein, because cfRNA is released from many different cell and types of cell-containing samples (e.g., tissues) at different times across different individuals, it is challenging to capture the diversity among cfRNA expression profiles using only cfRNA expression profiles that have been observed in a limited number of individuals (e.g., cfRNA expression profile 142). Accordingly, the inventors have developed techniques that account for this diversity. In some embodiments, the techniques include generating an initial healthy expression profile component 147 using a plurality of RNA expression profiles 144 previously obtained from biological samples from healthy subjects. In some embodiments, the plurality of RNA expression profiles 144 includes an RNA expression profile for each of a plurality of cell types and/or each of a plurality of types of cell-containing samples. An RNA expression profile for a particular cell type may include RNA-seq data for cells of the particular type. An RNA expression profile for a particular cell-containing sample type may include RNA-seq data for cell-containing samples of that particular cell-containing sample type. The RNA-seq data may be obtained from one or more databases such as, for example, the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10), The Cancer Genome Atlas (TCGA) database (TCGA Research Network: www.cancer.gov/tcga.), the BioStudies database (www.ebi.ac.uk/biostudies/), and the European Nucleotide Archive (ENA) database (www.ebi.ac.uk/ena). By generating artificial cfRNA expression profiles representing different proportions of different cell and types of cell-containing samples, it is possible to generate artificial cfRNA expression data that more accurately and comprehensively represents the diversity among cfRNA expression profiles likely to be observed across different individuals at different times.

In some embodiments, the RNA expression profiles 144 includes an RNA expression profile for each of a plurality of cell types. For example, as shown in FIG. 1D, the RNA expression profiles 144 include RNA expression profiles for cell type 1 144-1 through cell type N 144-2, where N is any suitable number as aspects of the technology described herein are not limited in this respect. For example, N may be at least 1, at least 2, at least 3, at least 4, at least 5, at least 6, at least 7, at least 8, at least 9, at least 10, at least 20, at least 30 or at least any other suitable number of cell types. Additionally, or alternatively, N may be at most 5, at most 6, at most 7, at most 8, at most 9, at most 10, at most 11, at most 12, at most 13, at most 14, at most 15, at most 20, at most 30, at most 40, at most 50, or at most any other suitable number of cell types. It should be appreciated that any of the above-listed upper bounds may be coupled with any of the above-listed lower bounds. In some embodiments, the cell types include any suitable, non-tumor cell types such as, for example, macrophages, monocytes, granulocytes, fibroblasts, endothelium, lymphocytes, epithelium, hepatocytes, stromal cells, myeloid cells, and platelets as aspects of the technology described herein are not limited in this respect.

In some embodiments, the RNA expression profiles 144 include an RNA expression profile for each of a plurality of types of cell-containing samples. For example, as shown in FIG. 1D, the RNA expression profiles 144 include RNA expression profiles for cell-containing sample type 1 144-3 through cell-containing sample type M 144-4, where M is any suitable number, as aspects of the technology described herein are not limited in this respect. For example, M may be at least 1, at least 2, at least 3, at least 4, at least 5, at least 6, at least 7, at least 8, at least 9, at least 10, at least 20, at least 30 or at least any other suitable number of cell types. Additionally, or alternatively, M may be at most 5, at most 6, at most 7, at most 8, at most 9, at most 10, at most 11, at most 12, at most 13, at most 14, at most 15, at most 20, at most 30, at most 40, at most 50, or at most any other suitable number of cell types. It should be appreciated that any of the above-listed upper bounds may be coupled with any of the above-listed lower bounds. In some embodiments, the types of cell-containing samples include any suitable types of cell-containing samples such as, for example, tissue, peripheral blood mononuclear cell (PBMC) and whole blood.

In some embodiments, the RNA expression profiles 144 are obtained from tissue and cell expression data store 143, from a sequencing platform (e.g., sequencing platform 102), by sequencing biological samples previously obtained from the healthy subjects, or in any other suitable way, as aspects of the technology described herein are not limited in this respect. In some embodiments, the data store 143 stores RNA expression profiles sorted by cell and tissue type. In some embodiments, the RNA expression profiles are sorted after being obtained by sequencing the biological sample(s). The biological samples from which the RNA expression profiles 144 were previously obtained may include any suitable type of biological sample such as any of the biological samples described in the section “Biological Samples.”

At act 145, a respective proportion is determined for each of the RNA expression profiles 144. In some embodiments, determining the proportions includes sampling the proportions from a distribution. For example, the proportions may be sampled from a Dirichlet distribution. The Dirichlet distribution may be parameterized by a vector α, which regulates the skewness of probabilities of each dimension in the Dirichlet distribution. In some embodiments, α is fixed to be the same value across all dimensions. For example, α may be fixed to 1 across all dimensions, which allows for uniform sampling of the space of all possible concentrations. Additionally or alternatively, the proportions may be sampled from a Gamma distribution. If proportions are drawn from any distribution other than the Dirichlet, the values may be renormalized to sum up to 1.

At act 146, the RNA expression profiles 144 are combined using the determined proportions to obtain an initial healthy expression profile component 147. In some embodiments, combining the RNA expression profiles 144 includes determining a weighted sum of the RNA expression profiles 144 using the determined proportions. For example, the weighted sum may be determined using Equation 2:

$${E}_{initial} = {\sum }_{i = 1}^{n} {ω}_{i} {E}_{Expression Profile , i} \qquad ( Equation 2 )$$

where Eᵢₙᵢₜᵢₐₗ represents the initial healthy expression profile component, E_{Expression Profile,i} represents the ith RNA expression profile, ωᵢ represents the proportion determined for the ith RNA expression profile, and n represents the total number of RNA expression profiles 144.

At act 148, the cfRNA expression profile 142 and the initial healthy expression profile component 147 are combined to obtain the healthy expression profile component 132. In some embodiments, this includes determining a weighted sum of the cfRNA expression profile 142 and the initial healthy expression profile component 147. For example, the weighted sum may be determined using Equation 3:

$${E}_{Healthy} = {w}_{cfRNA} {E}_{cfRNA} + {w}_{initial} {E}_{initial} \qquad ( Equation 3 )$$

where E_{Healthy} represents the healthy expression profile component 132, E_{cfRNA} represents the cfRNA expression profile 142, Eᵢₙᵢₜᵢₐₗ represents the initial healthy expression profile component, w_{cfRNA} represents the weight applied to the cfRNA expression profile, and wᵢₙᵢₜᵢₐₗ represents the weight applied to the initial healthy expression profile component 147. In some embodiments, the weights (wᵢₙᵢₜᵢₐₗ and w_{cfRNA}) are determined using any suitable techniques, as aspects of the technology described herein are not limited in this respect. For example, at least one of the weights may be determined by sampling from a distribution, such as a uniform distribution, a log-normal distribution, or any other suitable distribution. The lower bound of the uniform distribution may range between 0.4 and 0.8. The upper bound of the uniform distribution may range between 0.7 and 1.0. For example, the uniform distribution may range from 0.4 to 1.0, 0.5 to 1.0, 0.6 to 1.0, 0.7 to 1.0, 0.8 to 1.0, 0.9 to 1.0, 0.4 to 0.9, 0.5 to 0.9, 0.6 to 0.9, 0.7 to 0.9, or 0.8 to 0.9. For example, the weight w_{cfRNA} may be sampled from the uniform distribution: Uniform (0.7, 1.0). In some embodiments, weight wᵢₙᵢₜᵢₐₗ may be (1−w_{cfRNA}).

FIG. 1E is a diagram of an illustrative technique for generating a tumor expression profile component 134 used for generating an artificial cfRNA expression profile, according to some embodiments of the technology described herein.

In some embodiments, the tumor expression profile component 134 is determined based on a tumor expression profile 152 previously obtained from a tumor sample. In some embodiments, the tumor expression profile 152 is obtained from tumor expression data store 151, from a sequencing platform (e.g., sequencing platform 102), and/or by sequencing a tumor sample. The tumor sample may include any suitable tumor sample such as any of the tumor samples described in the section “Biological Samples,” as aspects of the technology described herein are not limited in this respect. For example, the tumor sample may include tumor samples coming from a cancer of the same type of the cancer being predicted. For example, when predicting whether the subject has breast cancer, the tumor sample may come from breast cancer samples.

As described herein, an expression profile, such as tumor expression profile 152, may indicate an expression level for each of a plurality of genes. In some embodiments, an expression level is measured based on read count, which refers to the number of reads that align to the particular gene. Accordingly, as shown in FIG. 1E, the tumor expression profile 152 may include a read count for each of a plurality of genes.

In some embodiments, the counts are used to determine a respective sampling probability for each of the plurality of genes. The determined sampling probabilities 154 may be included in a vector \vec{p} of sampling probabilities. In some embodiments, a sampling probability pᵢ of a particular gene (e.g., the ith gene) is determined based on the count cᵢ indicated for the gene and the total number of counts across all of the genes. For example, the sampling probability pᵢ may be determined using Equation 4:

$${p}_{i} = \frac{{c}_{i}}{{\sum }_{k = 1}^{n} {c}_{k}} \qquad ( Equation 4 )$$

where n is the total number of genes. Accordingly, in some embodiments the vector

$$\vec{p} = ( \frac{{c}_{1}}{{\sum }_{k = 1}^{n} {c}_{k}} , \frac{{c}_{2}}{{\sum }_{k = 1}^{n} {c}_{k}} , … \frac{{c}_{n}}{{\sum }_{k = 1}^{n} {c}_{k}} ) .$$

At act 155, reads are sampled according to the sampling probabilities 154, where each of the reads corresponds to a particular gene in the tumor expression profile 152. Each sample is a realization of a multinomial distribution with the probability vector (\vec{p}) over genes (coordinates), with a number of trials (reads) being determined as described herein. The resulting vector represents the expression profile of the tumor in counts. As described herein, the counts can be normalized (e.g., expressed in transcripts per million).

In some embodiments, the number of reads (trials) depends on the (a) a median coverage of sequencing of cfRNA samples, and (b) the proportion γ determined as described herein with respect to FIG. 1C. For example, the number of reads may be determined using Equation 5:

$$Number = Coverage \times γ \qquad ( Equation 5 )$$

For example, the median coverage may be between 10,000,000 and 50,000,000. For example, the median coverage may be 30,000,000. In some embodiments, the number of reads sampled is a constant. For example, the number of reads sampled may be 40,000,000

The sampled reads 156 are used to generate the tumor expression profile component at act 157. In some embodiments, this includes summing the reads sampled for each gene to determine an updated read count for each gene. In some embodiments, the updated count for each gene is converted to a number representative of a particular unit. In embodiments, a count as described herein is expressed as a normalized count, such as e.g. a count expressed in transcripts per million (TPM). For example, the updated count for each gene may be converted to a number representative of transcripts per million (TPM). For example, the updated count xᵢ of a particular gene i may be converted to TPM using Equation 6, and the TPM number used as the count for the gene:

$${x}_{i , TPM} = \frac{{x}_{i}}{( {\sum }_{k = 1}^{n} {x}_{k} ) \times {10}^{6}} \qquad ( Equation 6 )$$

Rather than simply using the tumor expression profile 152 as the tumor expression profile component, technique 150 allows for the generation of a diverse, yet biologically accurate tumor expression profile components, without requiring additional tumor expression data to be physically obtained from tumor samples.

FIG. 1F is a block diagram of an example system 160 for using a machine learning model, trained using artificial cfRNA expression data, to predict a characteristic of a subject, according to some embodiments of the technology described herein. System 160 includes computing device(s) 104 configured to have software 161 execute thereon to perform various functions in connection with using a trained machine learning model to predict a characteristic of a subject, training the machine learning model, and generating artificial cfRNA expression data. In some embodiments, the software 161 includes a plurality of modules. A module may include processor-executable instructions that, when executed by at least one computer hardware processor, cause the at least one computer hardware processor to perform function(s) of the module. Such modules are sometimes referred to herein as “software modules,” each of which includes processor-executable instructions configured to perform one or more acts of one or more processes, such as process 200 shown in FIG. 2A, process 220 shown in FIG. 2B, and process 240 shown in FIG. 2C.

The computing device(s) 104 may be operated by one or more user(s) 167. In some embodiments, the user(s) 167 may provide, as input to the computing device(s) 104 (e.g., by uploading one or more files, by interacting with a user interface of the computing device(s) 104, etc.) cfRNA expression data previously obtained from a blood sample from a subject. Additionally, or alternatively, the user(s) 167 may provide input specifying processing or other methods to be performed on the cfRNA expression data. Additionally, or alternatively, the user(s) 167 may access results of processing the cfRNA expression data. For example, the user(s) 167 may access a predicted characteristic of the subject.

In some embodiments, the condition prediction module 162 is configured to obtain cfRNA expression data previously obtained from a blood sample from a subject. For example, condition prediction module 162 may be configured to obtain the cfRNA expression data from the sequencing platform 102, cfRNA expression data store 141, and/or user(s) 167 (e.g., by the user uploading the cfRNA expression data and/or providing input specifying the cfRNA expression data).

In some embodiments, the condition prediction module 162 is configured to process the cfRNA expression data using a trained machine learning model to obtain a prediction indicative of a characteristic of the subject. For example, condition prediction module 162 may be configured to implement process 200 shown in FIG. 2A and/or at least a portion of illustrative technique 100. In some embodiments, the condition prediction module 162 obtains the trained machine learning model from machine learning model training module 163 and/or machine learning model data store 168.

In some embodiments, the machine learning model training module 163 is configured to train one or more machine learning modules to predict a respective characteristic of a subject. For example, the machine learning model training module 163 may obtain training data and/or validation data from artificial cfRNA data generation module 164, cfRNA expression data store 141, and/or user(s) 167 (e.g., by the user(s) uploading the training data). The machine learning model training module 163 may be configured to use the obtained training data and/or validation data to train and/or validate the one or more machine learning models to predict the characteristic(s) of the subject. For example, the machine learning model training module 163 may be configured to implement technique 110 shown in FIG. 1B and/or process 220 shown in FIG. 2B. In some embodiments, the machine learning model training module 163 may provide the trained machine learning model(s) to the machine learning model data store 168 for storage thereon. For example, the machine learning model training module 163 may provide the values of parameters of the machine learning model(s) to the machine learning model data store 168 for storage thereon.

In some embodiments, the artificial cfRNA data generation module 164 is configured to obtain (a) cfRNA expression data from cfRNA expression data store 141, (b) RNA expression data from tissue and cell expression data store 143, and/or (c) tumor expression data from tumor expression data store 151. Additionally, or alternatively, the artificial cfRNA data generation module 164 may obtain the cfRNA expression data, RNA expression data, and/or tumor expression data from sequencing platform 102 and/or user(s) 167.

In some embodiments, the artificial cfRNA data generation module 164 is configured to use the obtained cfRNA expression data, RNA expression data, and/or tumor expression data to generate artificial cfRNA expression data. For example, the artificial cfRNA data generation module 164 may be configured to implement technique 130 shown in FIG. 1C, technique 140 shown in FIG. 1D, technique 150 shown in FIG. 1E, and/or process 240 shown in FIG. 2C.

In some embodiments, report generation module 166 is configured to generate one or more reports relating to generating artificial cfRNA expression data. For example, the report may specify one or more expression profiles included in the artificial cfRNA expression data. Additionally, or alternatively, the report may specify one or more sources of expression data used to generate artificial cfRNA expression data. In some embodiments, report generation module 166 is configured to generate one or more reports relating to training a machine learning model to predict a characteristic of a subject. For example, the report may display results indicative of the performance of the machine learning model during training and/or validation. In some embodiments, report generation module 166 is configured to generate one or more reports relating to using a trained machine learning model to predict a characteristic of a subject. For example, the report includes an indication of one or more predicted characteristics of the subject. Additionally, or alternatively, the report indicates one or more recommended actions based on the predicted characteristic. For example, the report may include a recommendation to perform additional examination(s) (e.g., imaging, biopsy, etc.). Additionally, or alternatively, the report may include a recommendation to adjust the administration of a therapy to a subject.

In some embodiments, each of the data stores (e.g., cfRNA expression data store 141, tissue and cell expression data store 143, tumor expression data store 151, and machine learning model data store 168) includes any suitable type of data store (e.g., a flat file, a database system, a multi-file, etc.) and may store data in any suitable format, as aspects of the technology described herein are not limited in this respect. The data store(s) may be part of software 161 (not shown) or excluded from software 161, as shown in FIG. 1F.

As shown in FIG. 1F, software 161 also includes user interface module 165. User interface module 165 may be configured to generate a graphical user interface (GUI) through which user(s) 167 may provide input and view information generated by software 161. For example, in some embodiments, the user interface module 165 may be a webpage or web application accessible through an Internet browser. In some embodiments, the user interface module 165 may generate a GUI of an app executing on a user's mobile device. In some embodiments, the user interface module 165 may generate a number of selectable elements through which a user may interact. For example, the user interface module 165 may generate dropdown lists, checkboxes, text fields, or any other suitable element.

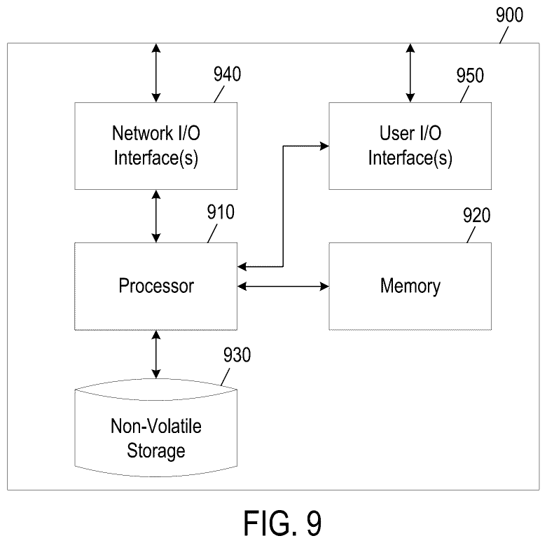

FIG. 2A is a flowchart of an illustrative process for using a machine learning model trained, using artificial cfRNA expression data, to predict a characteristic of a subject, according to some embodiments of the technology described herein. One or more of the acts of process 200 may be performed automatically by any suitable computing device(s). For example, act(s) may be performed by a laptop computer, a desktop computer, one or more servers, in a cloud computing environment, computing device(s) 104 described herein with respect to FIG. 1F, computing system 900 described herein with respect to FIG. 9, and/or in any other suitable way, as aspects of the technology described herein are not limited in this respect.

At act 202, cfRNA expression data is obtained for a blood sample from a subject. Techniques for obtaining a blood sample from a subject are described herein including at least with respect to technique 100 shown in FIGS. 1A and 1n the section “Biological Samples.” For example, the cfRNA expression data may include cfRNA expression data 103 shown in FIG. 1A.

At act 204, the cfRNA expression data is processed using a trained machine learning model to obtain an output indicative of a characteristic of the subject. In some embodiments, the trained machine learning model was trained using artificial cfRNA expression data comprising a plurality of artificial cfRNA expression profiles. Techniques for processing cfRNA expression data using a trained machine learning model are described herein including at least with respect to technique 100 shown in FIG. 1A. Techniques for training a machine learning model to predict a characteristic of a subject are described herein including at least with respect to technique 110 shown in FIG. 1B and process 220 shown in FIG. 2B. Techniques for generating artificial cfRNA expression data are described herein including at least with respect to technique 130 shown in FIG. 1C, technique 140 shown in FIG. 1D, technique 150 shown in FIG. 1E, and process 240 shown in FIG. 2C.

FIG. 2B is a flowchart of an illustrative process 220 for training a machine learning model to predict a characteristic of a subject using artificial cfRNA expression data, according to some embodiments of the technology described herein. One or more of the acts of process 220 may be performed automatically by any suitable computing device(s). For example, act(s) may be performed by a laptop computer, a desktop computer, one or more servers, in a cloud computing environment, computing device(s) 104 described herein with respect to FIG. 1F, computing system 900 described herein with respect to FIG. 9, and/or in any other suitable way, as aspects of the technology described herein are not limited in this respect.

At act 222, artificial cfRNA expression data is obtained. In some embodiments, the artificial cfRNA expression data includes (a) a first plurality of artificial cfRNA expression profiles representing cfRNA expression data from subjects having the particular characteristic, and (b) a second plurality of artificial cfRNA expression profiles representing cfRNA expression data from subjects not having the characteristic. Techniques for obtaining artificial cfRNA expression data are described herein including at least with respect to technique 110 shown in FIG. 1B. For example, the artificial cfRNA expression data may include artificial cfRNA expression data 111 shown in FIG. 1B.

At act 224, the machine learning model is trained to predict a characteristic of the subject using the artificial cfRNA expression data. Techniques for training a machine learning model to predict a characteristic of a subject are described herein including at least with respect to technique 110 shown in FIG. 1B.

FIG. 2C is a flowchart of an illustrative process for generating artificial cfRNA expression data, according to some embodiments of the technology described herein. One or more of the acts of process 240 may be performed automatically by any suitable computing device(s). For example, act(s) may be performed by a laptop computer, a desktop computer, one or more servers, in a cloud computing environment, computing device(s) 104 described herein with respect to FIG. 1F, computing system 900 described herein with respect to FIG. 9, and/or in any other suitable way, as aspects of the technology described herein are not limited in this respect.

At act 242, artificial cfRNA expression data is generated by generating a plurality of artificial cfRNA expression profiles. In some embodiments, generating a particular cfRNA expression profile comprises: (a) at act 244, generating a healthy expression profile component, (b) at act 252, generating a tumor expression profile component, and (c) at act 262, generating the particular artificial cfRNA expression profile by combining the healthy expression profile component and the tumor expression profile component. Acts 244 and 252 may be performed in parallel with one another, or sequentially. For example, act 244 may be performed prior to act 252, or act 252 may be performed prior to act 244.

At act 244, a healthy expression profile component is generated by performing act 246, act 248, and act 250. At act 246, a plurality of RNA expression profiles is obtained for biological samples from healthy subjects. In some embodiments, the plurality of RNA expression profiles includes a respective RNA expression profile for each of one or more cell types and/or one or more types of cell-containing samples. Techniques for obtaining a plurality of RNA expression profiles are described herein including at least with respect to technique 140 shown in FIG. 1D. For example, the plurality of RNA expression profiles may include RNA expression profiles 144 shown in FIG. 1D.

At act 248, an initial healthy expression profile component is generated by combining the plurality of RNA expression profiles. Techniques for generating an initial healthy expression profile component are described herein including at least with respect to act 146 of technique 140 shown in FIG. 1D.

At act 250, the healthy expression profile component is generated by combining the initial healthy expression profile component and a cfRNA expression profile obtained for a blood sample from a healthy subject. Techniques for combining the initial healthy expression profile component and the cfRNA expression profile are described herein including at least with respect to act 148 of technique 140 shown in FIG. 1D.

At act 252, a tumor expression profile component is generated by performing act 254, act 256, act 258, and act 260. At act 254, a tumor expression profile is obtained for a tumor sample. In some embodiments, the tumor expression profile includes a plurality of counts for a respective plurality of reads. Techniques for obtaining a tumor expression profile are described herein including at least with respect to technique 150 shown in FIG. 1E. For example, the tumor expression profile may include tumor expression profile 152.

At act 256, a respective sampling probability is determined for each of the plurality of genes using the plurality of counts included with the tumor expression profile. Techniques for determining sampling probabilities are described herein including at least with respect to act 153 of technique 150 shown in FIG. 1E.

At act 258, a plurality of reads is sampled using the sampling probabilities determined for each of the plurality of genes. In some embodiments, each of the plurality of sampled reads corresponds to a gene of the plurality of genes. Techniques for sampling a plurality of reads are described herein including at least with respect to act 155 of technique 150 shown in FIG. 1E.

At act 260, the tumor expression profile component is generated by summing, for each particular gene of the plurality of genes, a number of sampled reads corresponding to the particular gene. Techniques for generating the tumor expression profile are described herein including at least with respect to act 157 of technique 150 shown in FIG. 1E.

At act 262, the particular artificial cfRNA expression profile is generated by combining the healthy expression profile component and the tumor expression profile component. Techniques for combining the healthy expression profile component and the tumor expression profile component are described herein including at least with respect to act 135 of technique 130 shown in FIG. 1C.

### Example 1—Liver Metastasis

In this example, a machine learning model was trained to predict whether a subject has liver metastasis using cfRNA expression data previously obtained from a blood plasma sample from the subject.

The output of the trained machine learning model can be used for many different applications. For example, the output may be used to confirm and/or prompt the clinical diagnosis of liver metastasis. For example, when the output of the trained machine learning model indicates that the subject has liver metastasis, then one or more additional examinations may be performed to confirm and/or measure the extent to which the subject has liver metastasis. Such examinations may include ultrasound and/or biopsy. As a second example, the output may be used to inform treatment decisions.

### Training Data

Expression data was obtained for tumor, cell, and types of cell-containing samples listed in Table 1. The expression data included RNA-seq data for the tumor, cell, and cell-containing samples listed in Table 1. The RNA-seq data was obtained from databases including the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10) and The Cancer Genome Atlas (TCGA) database (TCGA Research Network: www.cancer.gov/tcga.).

The expression data was preprocessed using a plurality of technical and biological quality control techniques. The technical quality control techniques included processing the training data using FastQC (Andrews S. (2010). FastQC: a quality control tool for high throughput sequence data.), FastQ Screen, SNP pileup, HLA matching, RseQC, and DNA contamination assessment. FastQ Screen is described by Wingett, Steven W., and Simon Andrews. (“FastQ Screen: A tool for multi-genome mapping and quality control.” F1000Research 7 (2018).), which is incorporated by reference herein in its entirety. RseQC is described by Wang, Liguo, Shengqin Wang, and Wei Li. (“RSeQC: quality control of RNA-seq experiments.” Bioinformatics 28.16 (2012): 2184-2185.), which is incorporated by reference herein in its entirety. The results of the technical quality control techniques were aggregated into a report using MultiQC. MultiQC is described by Ewels, Philip, et al. (“MultiQC: summarize analysis results for multiple tools and samples in a single report.” Bioinformatics 32.19 (2016): 3047-3048.), which is incorporated by reference herein in its entirety.

After performing the data pre-processing techniques, expression data was renormalized to a set of 18,792 genes, including housekeeping genes. Example normalization techniques are described by Abbas-Aghababazadeh, Farnoosh, Qian Li, and Brooke L. Fridley (“Comparison of normalization approaches for gene expression studies completed with high-throughput sequencing.” PloS one 13.10 (2018): e0206312.), which is incorporated by reference herein in its entirety.

The expression data was further filtered to filter out tumor samples having a tumor purity outside the range: [0.2, 0.8], which represents tumor content relative to non-tumor content in the sample.

The expression data was used to generate artificial cfRNA expression profiles representing cfRNA expression data from subjects having liver metastasis and artificial cfRNA expression profiles representing cfRNA expression data from subjects not having liver metastasis. All artificial cfRNA expression profiles were generated by: (i) generating a healthy expression profile component, (ii) generating a tumor expression profile component, and (iii) combining the healthy expression profile component and the tumor expression profile component. The tumor expression profile component was generated using a tumor expression profile obtained from the tumor samples listed in Table 1. The healthy expression profile component was generated by combining healthy expression profiles obtained from the non-tumor cell and cell-containing samples listed in Table 1. For the artificial cfRNA expression profiles representing cfRNA expression data from subjects having liver metastasis, the healthy expression profile component was generated using at least one healthy expression profile from liver tissue. For the artificial cfRNA expression profiles representing cfRNA expression data from subjects not having liver metastasis, the healthy expression profile component was not generated using any expression profiles from liver tissue.

Table 1 also lists the ranges from which the proportions of each cell type, cell-containing sample type, and tumor were selected. The proportions of each cell type and cell-containing sample type were determined according to the techniques described herein including at least with respect to FIG. 1D and Equation 2. The tumor proportion was determined according to techniques described herein including at least with respect to FIG. 1C and Equation 1.

The expression data was used to generate artificial cfRNA expression profiles. Artificial cfRNA expression profile parameters were determined as shown in Table 1. Table 1 specifies: the sample class (liver metastasis or no liver metstasis), number of tumor samples and artificial cfRNA expression profiles generated (5,000), and the proportional ranges for various cell and types of cell-containing samples (e.g., whole blood, myeloid cells, tumor cells, etc.).

The training data included the artificial cfRNA expression profiles. 5,000 artificial expression profiles contained tumor expression data. 5,000 artificial expression profiles did not contain tumor expression data. The artificial cfRNA expression profiles were labeled with clinical annotations indicating whether or not the expression profile contained tumor expression data. The clinical annotations were used as ground truth.

The training data was used to train a LightGBM decision tree boosting machine learning model to predict whether the subject has liver metastasis. Specifically, the LightGBM decision tree boosting machine learning model was trained using the artificial cfRNA expression profiles generated for training. LightGBM is described by Ke, Guolin, et al. (“Lightgbm: A highly efficient gradient boosting decision tree.” Advances in neural information processing systems 30 (2017).), which is incorporated by reference herein in its entirety.

Feature selection was performed on the artificial cfRNA expression training data during optimization of the LightGBM decision tree boosting machine learning model to select a number of feature (e.g., genes). High dropout genes were excluded. Dropout genes included those genes having an expression of 0 in at least 80% of the samples. Genes for which the expression levels of the majority of real samples fell within (e.g., between 0.1 and 0.9 quantiles) the artificial distribution, showing a low KL divergence (30th and 70th percentile), were retained. 200 genes were selected. Feature selection involved generating cross-validated mixes. Each fold excluded one run with healthy plasmas. The number of differentially-expressed genes was reduced for each fold until there were less than 200 genes remaining in the intersection. Gene set enrichment analysis was performed to ensure the biological relevance of the obtained features. Table 2 lists the genes that were selected.

The model was controlled for predicting possible batches in the data instead of the target label. The annotations listed in the description were checked to determine if they explained model prediction better than the target label (liver metastasis) by comparing the AUC curves.

Table 3 lists the parameters of the trained model.

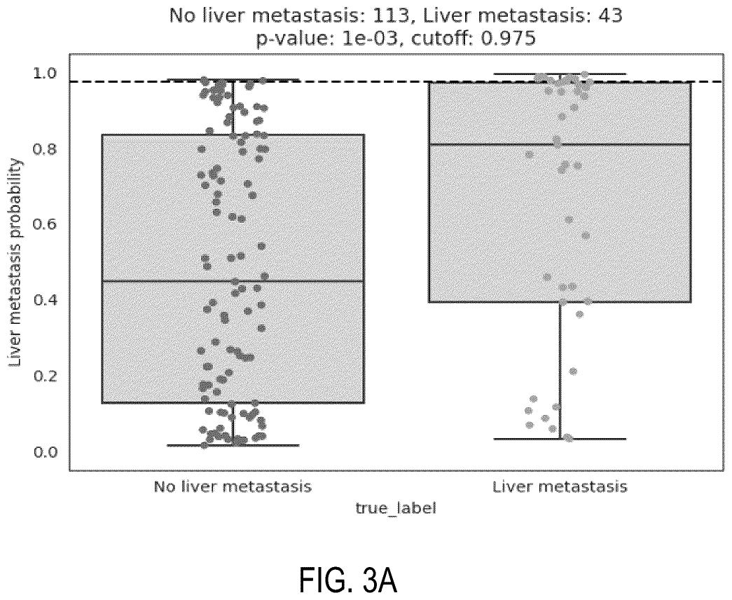

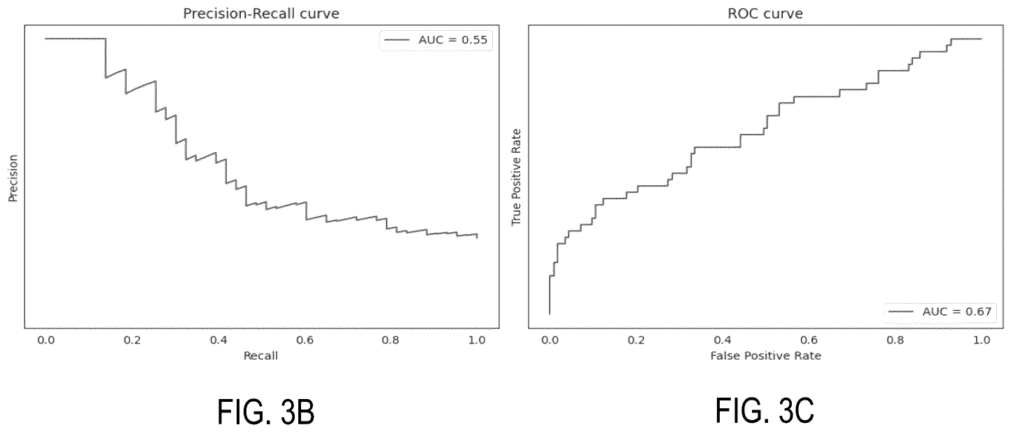

FIG. 3A, FIG. 3B, and FIG. 3C show that the trained machine learning model accurately distinguishes between subjects having liver metastasis and subjects not having liver metastasis. In particular, as shown in FIG. 3A, the trained machine learning model distinguishes between healthy subjects and subjects having liver metastasis with a p-value of 1e-03.

TABLE 1
describes the training data used to train the machine learning
model to predict whether a subject has liver metastasis.

| Sample Class | Liver Metastasis | No Liver Metastasis |
|---|---|---|
| Number and Types of | BRCA: 2,345 | BRCA: 2,345 |
| Tumor Samples Used for | LUAD: 1,106 | LUAD: 1,106 |
| Artificial Mixes | COAD: 632 | COAD: 632 |
| | PAAD: 334 | PAAD: 334 |
| | Number of samples = 4,417. | Number of samples = 4,417. |
| Number and Types of Cell, | Whole Blood: 18,884 | Whole Blood: 18,884 |
| Cell-Containing, and | Myeloid Cells: 1,623 | Myeloid Cells: 1,623 |
| Tumor Samples Used for | Stromal Cells: 1,370 | Stromal Cells: 1,370 |
| Artificial Mixes | Lymphoid Cells: 4,046 | Lymphoid Cells: 4,046 |
| | Epithelium: 39 | Epithelium: 39 |
| | Platelets: 763 | Platelets: 763 |
| | Liver Tissue: 110 | |
| Number of Artificial cfRNA | 5,000 | 5,000 |
| Expression Profiles | | |
| Generated for Training | | |
| Cell, Cell-Containing | Whole Blood: [0, 0.6], | Whole Blood: [0, 0.6], |
| Sample, and Tumor Types | Myeloid Cells: [0. 0.5], | Myeloid Cells: [0. 0.3], |
| (and the Lower and Upper | Stromal Cells: [0, 0.2], | Stromal Cells: [0, 0.3], |
| Bounds of their | Platelets: [0, 0.8], Lymphoid | Platelets: [0, 0.5], Lymphoid |
| Proportions) for Which | Cells: [0, 0.3], Epithelium: | Cells: [0, 0.3], Epithelium: |
| Expression Profiles Were | [0, 0.3], Tumor: [5e−3, 1e−1], | [0, 0.3], Tumor: [5e−2, 1e−1] |
| Obtained and Used to | Liver Tissue: [0, 1] | |
| Generate Artificial cfRNA | | |
| Expression Data | | |
| Plasma Proportion Lower | [0.7, 0.9] | [0.7, 0.9] |
| and Upper Bounds | | |

TABLE 2
lists the genes selected during feature selection
performed for the liver metastasis model.

| | |
|---|---|
| Genes | HRG, SORD, PROZ, PRMT2, EHHADH, MARCH8, EIF4G1, CECR5, |
| | MRPS15, PMVK, GATAD2A, TUBB, TCEAL8, TCEAL4, BTBD1, |
| | ARG1, COQ9, GNAI2, MAN2B1, ADD1, TRAF7, DHRS4L2, RHOQ, |
| | ALAS1, ISCA1, UBR4, SLC17A4, FAM207A, KHSRP, PRKACA, |
| | KIAA0930, BRE, LRRC41, SNAP29, WDR45, PABPC1, GOLGA5, |
| | C17orf89, UBQLN4, COPG1, TNS2, SMIM12, MRPL37, UBALD2, |
| | CHPT1, TMED2, CSTB, AIDA, CTNNA1, TKFC, ZNF768, C16orf45, |
| | UCK2, FAM129B, VAMP8, FILIP1L, YY1, CTDSP1, VTI1B, C1orf43, |
| | AK1, CARHSP1, ACTN4, DHTKD1, SDR39U1, ATPAF1, CAMKK2, |
| | RAB3GAP1, AHNAK, GNAI1, YBX1, SH3GL1, FBXO22, SHARPIN, |
| | MRPL20, DESI1, ELAVL1, PPP1CA, TSPO, RNPS1, HMGB2, UBE2I, |
| | HMGA1, LHPP, EPN1, SEC23A, NBPF14, RBFA, GLRX2, ZMYND11, |
| | PTPN11, NDST1, TMEM222, ID1, PPARA, ARL2BP, SLC30A1, VAMP5, |
| | CARM1, EMC6, YTHDF2, ABHD14B, CITED2, SEL1L3, NFU1, |
| | EFTUD2, STUB1, MRPL53, ZC3H7B, NINJ2, USP14, SNRPC, TAF13, |
| | CTSC, TCF3, DIABLO, TNFSF14, HMGN3, PINK1, NDUFA13, TP53, |
| | HSF1, FXYD6, H2AFV, DHDDS, SRSF1, CDK16, IRAK1, DNM2, |
| | ARAF, PDK2, RNF5, RNF40, SAE1, SMAD3, GLYR1, TNS3, ZFPL1, |
| | UBE2J2, TAF6, ASCC1, NUP62, SMARCB1, DAZAP1, GUK1, DAPK3, |
| | ATP5D, FIBP, AP3S1, NEK6 |

TABLE 3
lists the parameters of the trained liver metastasis ML model.

| Parameter | Value |
|---|---|
| boosting_type | gbdt |
| feature_fraction | 0.06688717884946171 |
| learning_rate | 0.0016454112619556668 |
| max_depth | 7 |
| min_child_samples | 500 |
| n_estimators | 205 |
| num_leaves | 128 |
| reg_alpha | 2.490664040420922 |
| reg_lambda | 0.005403806645967165 |
| subsample | 0.12670786813216495 |
| subsample_freq | 2 |

### Example 2—Breast Cancer

In this example, a machine learning model was trained to predict whether a subject has breast cancer using cfRNA expression data previously obtained from a blood plasma sample from the subject.

The output of the trained machine learning model can be used for many different applications. As a first example, the output may be used to confirm and/or prompt the clinical diagnosis of breast cancer. For example, when the output of the trained machine learning model indicates that the subject has breast cancer, then one or more additional examinations may be performed to confirm and/or measure the extent to which the subject has breast cancer. Such examinations may include mammography and/or biopsy. As a second example, the output may be used as a surrogate endpoint in drug clinical trials. For example, when the output of the machine learning model indicates that the subject does not have (or no longer has) breast cancer, then therapy administration may be canceled.

### Training Data

Expression data was obtained for tumor, cell, and types of cell-containing samples listed in Table 4. The expression data included RNA-seq data for the tumor, cell, and cell-containing samples listed in Table 4. The RNA-seq data was obtained from databases including the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10) and The Cancer Genome Atlas (TCGA) database (TCGA Research Network: www.cancer.gov/tcga.).

The expression data was preprocessed using a plurality of technical and biological quality control techniques. The technical quality control techniques included processing the training data using FastQC (Andrews S. (2010). FastQC: a quality control tool for high throughput sequence data.), FastQ Screen, SNP pileup, HLA matching, RseQC, and DNA contamination assessment. FastQ Screen is described by Wingett, Steven W., and Simon Andrews. (“FastQ Screen: A tool for multi-genome mapping and quality control.” F1000Research 7 (2018).), which is incorporated by reference herein in its entirety. RseQC is described by Wang, Liguo, Shengqin Wang, and Wei Li. (“RSeQC: quality control of RNA-seq experiments.” Bioinformatics 28.16 (2012): 2184-2185.), which is incorporated by reference herein in its entirety. The results of the technical quality control techniques were aggregated into a report using MultiQC. MultiQC is described by Ewels, Philip, et al. (“MultiQC: summarize analysis results for multiple tools and samples in a single report.” Bioinformatics 32.19 (2016): 3047-3048.), which is incorporated by reference herein in its entirety.

After performing the data pre-processing techniques, expression data was renormalized to a set of 17,000 genes, including housekeeping genes. Example normalization techniques are described by Abbas-Aghababazadeh, Farnoosh, Qian Li, and Brooke L. Fridley (“Comparison of normalization approaches for gene expression studies completed with high-throughput sequencing.” PloS one 13.10 (2018): e0206312.), which is incorporated by reference herein in its entirety.

The expression data was further filtered to filter out tumor samples having a tumor purity lower than 0.6, which represents tumor content relative to non-tumor content in the sample.

The expression data was used to generate artificial cfRNA expression profiles representing cfRNA expression data from subjects having breast cancer and artificial cfRNA expression profiles representing cfRNA expression data from subjects not having breast cancer. Artificial cfRNA expression profiles representing cfRNA expression data from subjects having breast cancer were generated by: (i) generating a healthy expression profile component, (ii) generating a tumor expression profile component, and (iii) combining the healthy expression profile component and the tumor expression profile component. The tumor expression profile component was generated using a tumor expression profile obtained from the tumor samples listed in Table 4. The healthy expression profile component was generated by combining healthy expression profiles obtained from the non-tumor cell and cell-containing samples listed in Table 4. Artificial cfRNA expression profiles representing cfRNA expression data from subjects not having breast cancer (e.g., healthy subjects) were generated by: (i) generating a healthy expression profile component (as described above), (ii) using the healthy expression profile component as the artificial cfRNA expression profile.

Table 4 also lists the ranges from which the proportions of each cell type, cell-containing sample type, and tumor were selected. The proportions of each cell type and cell-containing sample type were determined according to the techniques described herein including at least with respect to FIG. 1D and Equation 2. The tumor proportion was determined according to techniques described herein including at least with respect to FIG. 1C and Equation 1.

The training data included the artificial cfRNA expression profiles. The artificial cfRNA expression profiles were labeled with clinical annotations that indicated whether or not the expression profile contained tumor (e.g., breast cancer) expression data. The clinical annotations were used as ground truth.

The training data was used to train a LightGBM decision tree boosting machine learning model to predict whether or not a subject has breast cancer given cfRNA expression data obtained for the subject. Specifically, the LightGBM decision tree boosting machine learning model was trained using the artificial cfRNA expression profiles generated for training.

Feature selection was performed on the artificial cfRNA expression training data during optimization of the LightGBM decision tree boosting machine learning model to select a number of feature (e.g., genes). Genes with low expression in tumor (95^{th} percentile expression in tumor is less than 50 TPM) were excluded. Transcripts capturing patient-specific features and ribosomal genes were excluded. High dropout genes were excluded. Dropout genes included those genes having an expression of 0 in at least 80% of the samples. Genes for which the expression levels of the majority of real samples fell within (e.g., between 0.1 and 0.9 quantiles) the artificial distribution, showing a low KL divergence (30^{th} and 70^{th} percentile), were retained. 150 genes were selected. Feature selection involved generating cross-validated mixes. Each fold excluded one run with healthy plasmas. Differentially-expressed genes (DEGs) common for all folds were selected (e.g., less than 2,000 genes for each fold with lowest p-value, p-value <0.05) using the Mann-Whitney test. The DEGs were filtered using real cfRNA samples. The number of DEGs was reduced for each fold until there were less than 200 genes remaining in the intersection. Gene set enrichment analysis was performed to ensure the biological relevance of the obtained features. Table 5 lists the genes that were selected.

The model was controlled for predicting possible batches in the data instead of the target label. The annotations listed in the description were checked to determine if they explained model prediction better than the target label (breast cancer) by comparing the AUC curves.

Table 6 lists the parameters of the trained model.

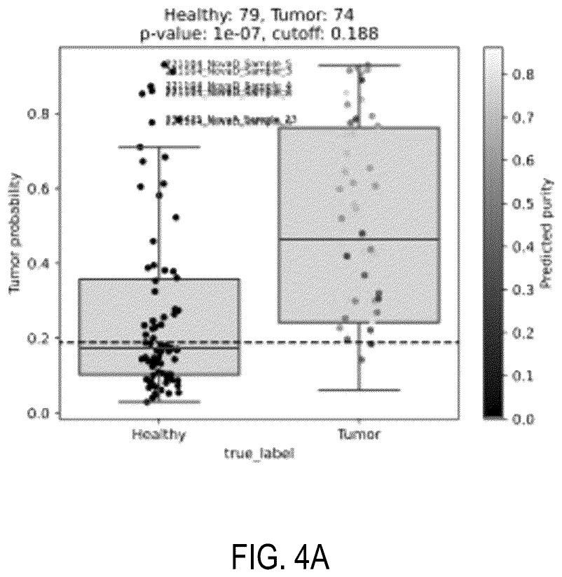

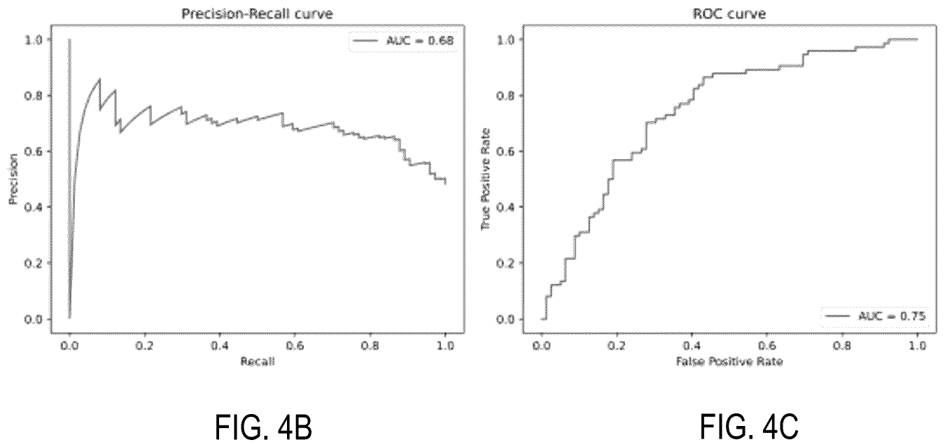

FIG. 4A, FIG. 4B, and FIG. 4C show that the trained machine learning model accurately distinguishes between subjects having breast cancer and subjects not having breast cancer. In particular, as shown in FIG. 4A, the trained machine learning model distinguishes between healthy subjects and subjects having breast cancer with a p-value of 1e-07.

TABLE 4
describes the training data used to train the machine learning
model to predict whether a subject has breast cancer.

| Sample Class | Breast Cancer | No Breast Cancer |
|---|---|---|
| Number and Types of | BRCA: 2,345 | No Tumor Samples |
| Tumor Samples Used for | | |
| Artificial Mixes | | |
| Number and Types of Cell, | Whole Blood: 18,884 | Whole Blood: 18,884 |
| Cell-Containing, and | Myeloid Cells: 1,623 | Myeloid Cells: 1,623 |
| Tumor Samples Used for | Stromal Cells: 1,370 | Stromal Cells: 1,370 |
| Artificial Mixes | Lymphoid Cells: 4,046 | Lymphoid Cells: 4,046 |
| | Epithelium: 39 | Epithelium: 39 |
| | Platelets: 763 | Platelets: 763 |
| Number of Artificial cfRNA | 500 | 500 |
| Expression Profiles | | |
| Generated for Training | | |
| Cell, Cell-Containing | Whole Blood: [0, 0.6], | Whole Blood: [0, 0.6], |
| Sample, and Tumor Types | Myeloid Cells: [0, 0.5], | Myeloid Cells: [0, 0.5], |
| (and the Lower and Upper | Stromal Cells: [0, 0.2], | Stromal Cells: [0, 0.2], |
| Bounds of their | Platelets: [0, 0.8], Lymphoid | Platelets: [0, 0.8], Lymphoid |
| Proportions) for Which | Cells [0, 0.3], Hepatocytes: | Cells [0, 0.3], Hepatocytes: |
| Expression Profiles Were | [0, 0.1], Epithelium: [0, 0.3], | [0, 0.1], and Epithelium: [0, |
| Obtained and Used to | BRCA Tumor [5e−2, 1e−1] | 0.3]. |
| Generate Artificial cfRNA | | |
| Expression Data | | |
| Plasma Proportion Lower | [0.7, 0.9] | [0.7, 0.9] |
| and Upper Bounds | | |

TABLE 5
lists the genes selected during feature selection
performed for the breast cancer model.

| | |
|---|---|
| Genes | HIPK1, TCEAL4, RASAL2, FZD1, RET, CTNNB1, LRRC41, CDCA8, |
| | CCDC88C, ZNF768, PLEKHB2, MACC1, UBR4, NKIRAS2, ARPC4, |
| | CSRP1, RREB1, MTSS1, PPP2R4, GNL3L, OCRL, COPA, ATN1, |
| | NUDT3, TPX2, GOLGA2, DR1, CCND1, CRK, CLTC, SYNPO, WDTC1, |
| | TMEM65, TNS3, DVL3, DNAL4, KANSL1L, MRPL37, NFATC2IP, |
| | SNX18, RMND5A, TAOK2, NFX1, DLC1, RMND5B, FANCI, POGK, |
| | MAFB, VPS26B, PPP1CB, GNA12, RAB11FIP3, FYCO1, DAPK3, |
| | PPP2CB, RGS3, SNAP29, CRKL, CLINT1, CNTROB, MSRA, YES1, |
| | CDK5RAP2, GGNBP2, NES, PEX26, USP39, UBE2V1, AP2S1, |
| | GRIPAP1, KIF1B, VDR, KLF9, NT5C2, AMOTL1, RBM12, SH3BP5, |
| | NOL4L, BTBD2, ABCD1, RAB5C, KCTD9, SLC24A1, RBBP6, UBE2R2, |
| | TMED8, NBEAL2, MAVS, GLYR1, VCP, GAB2, RECQL5, NPEPPS, |
| | RNF115, ARAF, CENPB, ANKHD1, CHTF8, LAP3, PLSCR4, FAM127A, |
| | WBP1L, SVIL, WDR26, KDM3B, CTTNBP2NL, ABAT, PJA2, DUSP3, |
| | KHNYN, GBF1, BAP1, PLEKHG2, AGPAT3, DPM2, CYYR1, USP22, |
| | C2orf68, TMEM60, TK2, SKI, AK1, MEF2D, WDR13, UBN1, YWHAG, |
| | RIMKLB, SRGAP2B, ASRGL1, TBC1D10B, SFT2D2, TSPYL2, TAF13, |
| | TPGS2, DUSP5, ZMIZ2, PLEKHJ1, EDEM1, UBE2Q1, WSB2, PACS2, |
| | TBC1D17, FADD, PAFAH1B2, PBX2, SGMS1, UBALD1, METTL7A, |
| | YIPF6, TRIM8 |

TABLE 6
lists the parameters of the trained breast cancer ML model.

| Parameter | Value |
|---|---|
| boosting_type | dart |
| feature_fraction | 0.07524359482355654 |
| learning_rate | 0.019753611170716768 |
| max_depth | 4 |
| min_child_samples | 85 |
| n_estimators | 978 |
| num_leaves | 16 |
| reg_alpha | 0.5598418356048347 |
| reg_lambda | 1.0738576432290931e−08 |
| subsample | 0.941831034358922 |
| subsample_freq | 4 |

### Example 3—Chronic Lymphocytic Leukemia (CLL)

Chronic lymphocytic leukemia (CLL) is a heterogeneous disease of B cell lymphocytes with diverse molecular and phenotypic characteristics. Precision medicine approaches have shown promise for personalized treatment selection and disease monitoring in CLL patients. While conventional profiling uses flow cytometry (FC) to identify surface protein patterns, it cannot detect gene signatures for treatment response and resistance, or identify malignant B cell receptor (BCR) clonotypes. The techniques described herein address this limitation of conventional profiling techniques and can be used to characterize and monitor CLL using plasma cell-free RNA (cfRNA).

In this example, a machine learning model (a “CLL-specific ML model”) was trained to predict the malignant B cell fraction from cfRNA expression data previously obtained from a blood plasma sample from the subject. This example includes the sections: “Methods,” “Signals from Blood and Tissue Cells in cfRNA are Associated with Treatment,” “Matching of Dominant BCR Clonotypes Between PBMC RNA-seq and cfRNA-seq,” “Correlation Between cfRNA-based ML Model Predictions and Malignant B Cell Fractions,” “Training,” and “CLL Detection.”

### Methods

Whole blood was collected from 34 CLL patients: 20 treatment-naive, 12 on different types of treatments, and 2 post-treatment. Abundance of cfRNA transcripts from cell type-specific signatures was analyzed using single-sample gene set enrichment analysis (ssGSEA). BCR repertoire reconstruction and deconvolution of major cell populations were performed from peripheral blood mononuclear cell (PBMC) RNA-seq and plasma cfRNA-seq. The fraction of each clonotype was calculated based on sequencing coverage. Proportions of malignant B cells were predicted from cfRNA using the CLL-specific ML model. The CLL-specific ML model was trained according to the techniques described in the section of Example 3 entitled “Training.” Flow cytometry staining panels were used to detect malignant B cells and profile blood immune populations from PBMCs. Somatic mutation calling was performed using the BostonGene Tumor Portrait assay on DNA extracted from B cells or PBMCs as the source of malignant cells and from sorted or enriched T cells as the source of normal cells. Tumor-specific mutations were called from cfRNA fractions using Pisces 5.2.

Signals from Blood and Tissue Cells in cfRNA are Associated with Treatment

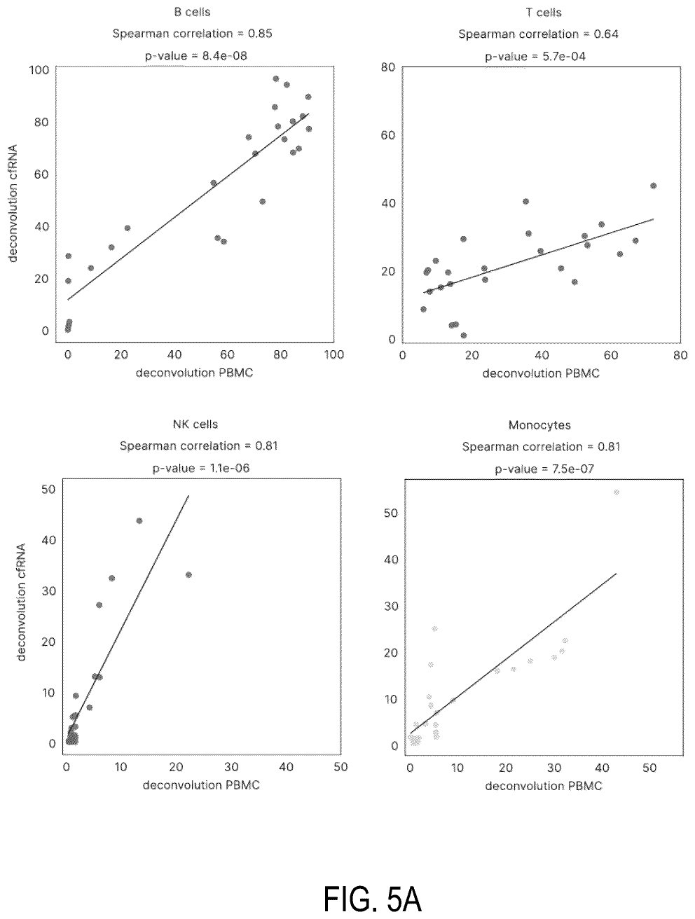

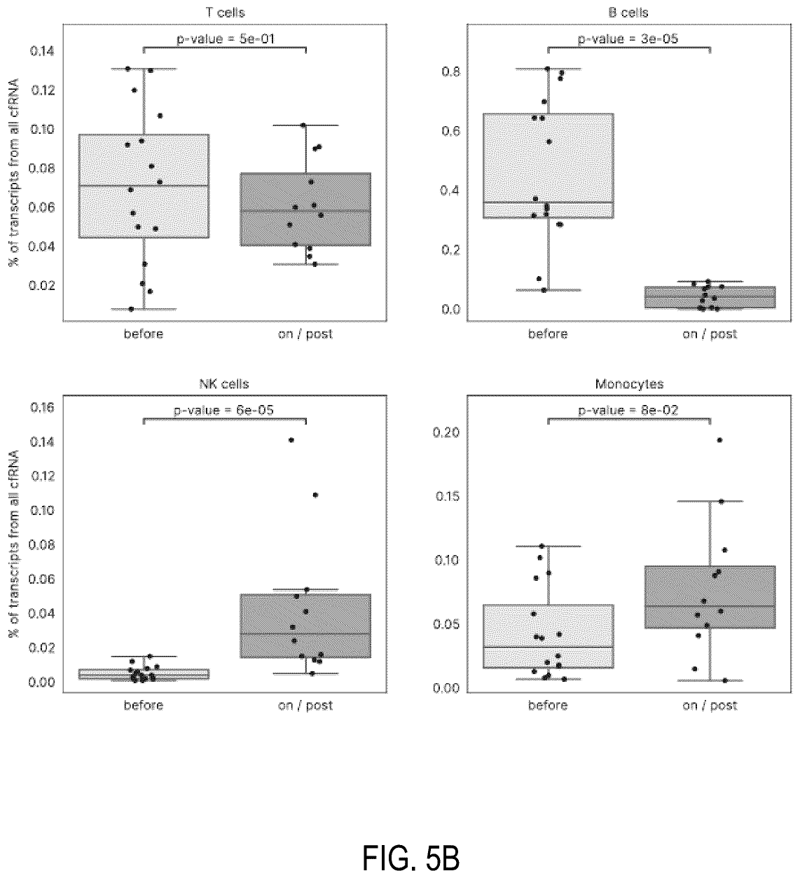

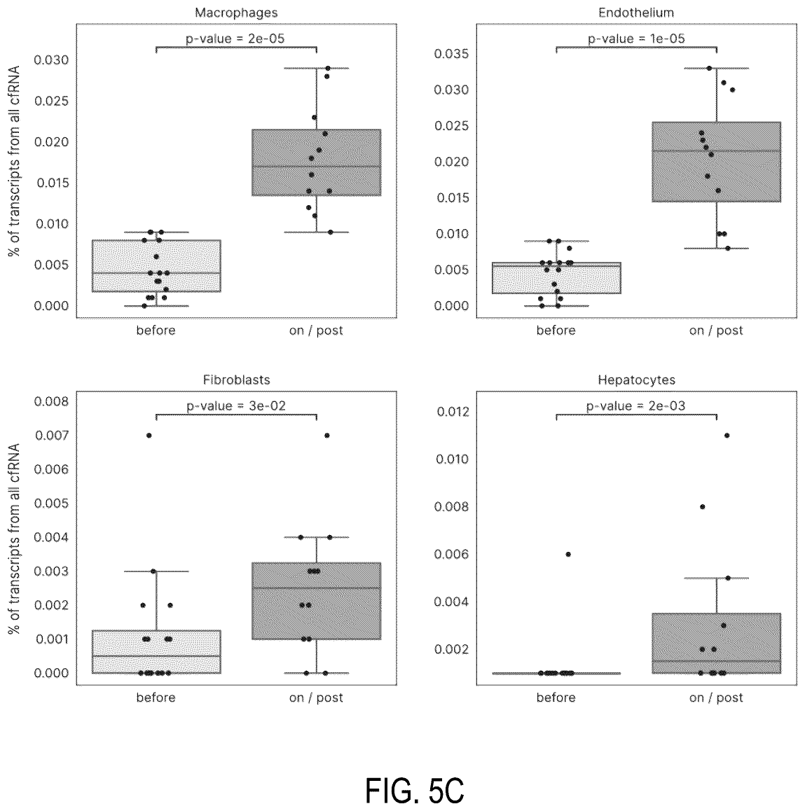

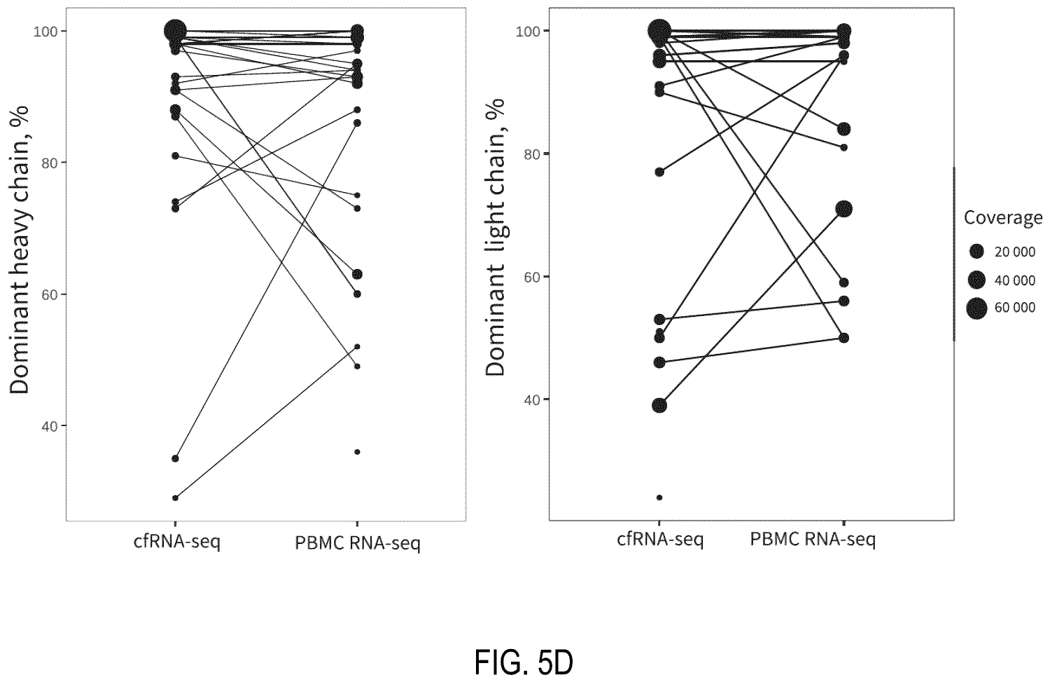

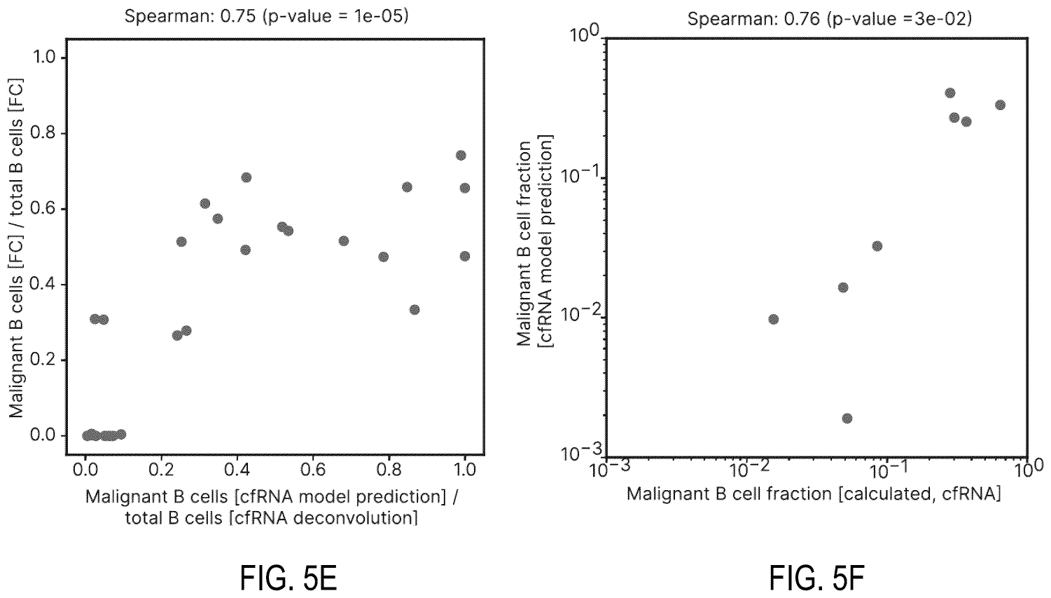

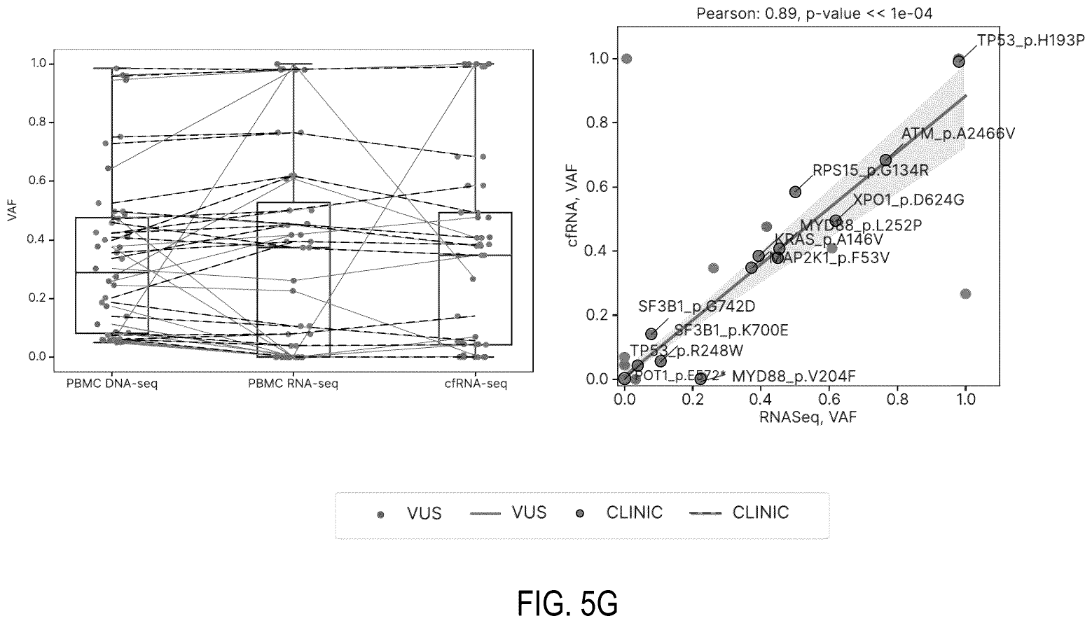

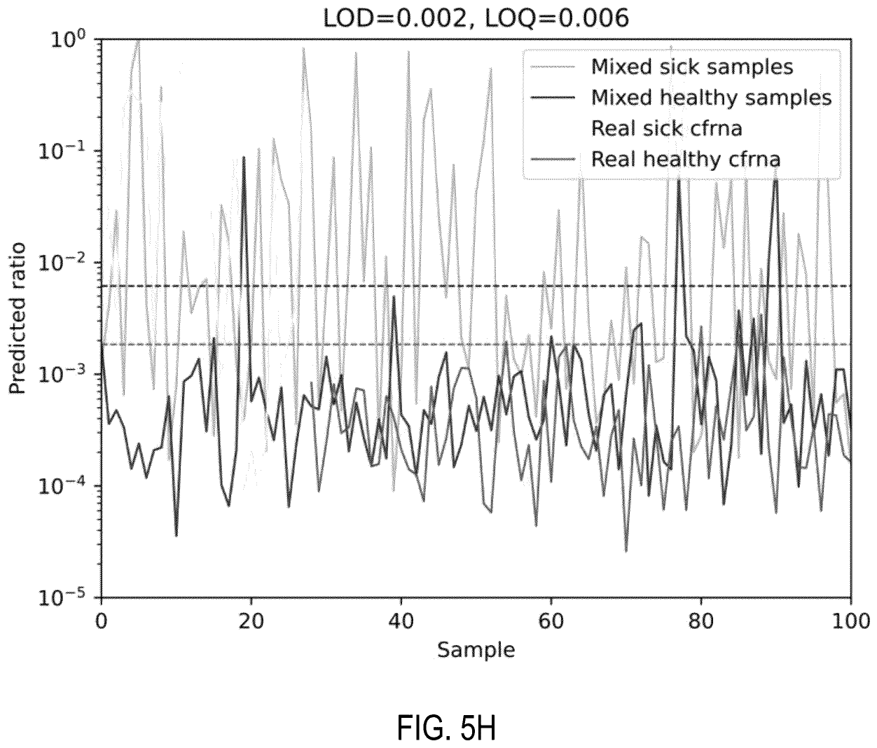

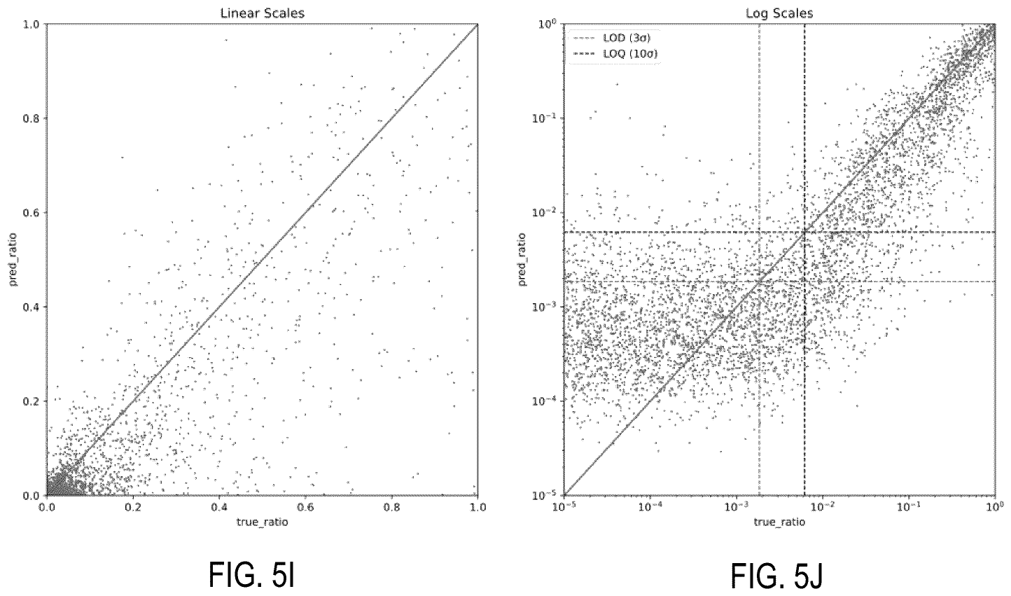

As shown in FIG. 5A, levels of four immune cell types (e.g., B cells, T cells, NK cells, and monocytes) defined with cfRNA-based deconvolution correlate with those identified with PBMC RNA-seq-based deconvolution.

cfRNA-based deconvolution and ssGSEA revealed cell composition dynamics relevant for treatment monitoring. As shown in FIG. 5B, significant changes were detected starting from the first weeks of therapy: decreased B cell levels (P=0.00003, Mann-Whitney U test) on- and post-treatment, increased NK cells (P=0.00006), and no significant changes in T cells or monocytes. Moreover, as shown in FIG. 5C, some tissue cell populations such as macrophages (P=0.00002), endothelium (P=0.00001), fibroblasts (P=0.03), and hepatocytes (P=0.002) showed significantly increased levels on- and post-treatment.

Matching of Dominant BCR Clonotypes Between PBMC RNA-Seq and cfRNA-Seq

cfRNA-inferred dominant BCR clones for cases with high tumor cell fraction detected by FC were considered malignant. Among 20 patients with both cfRNA-seq and PBMC RNA-seq available, 18 patients had dominant clones matched by heavy chain CDR3 sequences and 17 patients had dominant clones matched by light chain CDR3 sequences. Comparison of the coverage of the BCR region for malignant clonotypes relative to protein-coding regions showed 2 times (P=0.008, Wilcoxon test) higher levels in cfRNA compared to PBMC RNA-seq indicating potential advantages for monitoring treatment, relapse, and minimal residual disease (MRD) using cfRNA. As shown in FIG. 5D, dominant BCR clonotypes were highly connected between cfRNA-seq and PBMC RNA-seq: 90% of cases had dominant clones with matching CDR3 sequences in the heavy chain and 84% in the light chain. The CDR3 sequences are represented with dots. Matched sequences in cfRNA-seq and PBMC RNA-seq are connected. The size of the dots represents sequencing coverage.

### Detection of Clinically Significant Tumor-Derived Mutations

As shown in FIG. 5G, tumor-derived mutations were successfully called from cfRNA transcriptome. 24 coding mutations were identified and confirmed by PBMC WES and RNA-seq. In FIG. 5G, VUS indicates variant of uncertain significance, CLINIC indicates clinically significant variant, and VAF indicates variant allele frequency Among the identified somatic variants, nine were clinically significant missense mutations in: SF3B1, LoF in TP53, ATM, and BIRC3, truncating mutations in the PEST domain of NOTCH1.

Correlation Between cfRNA-Based ML Model Predictions and Malignant B Cell Fractions

The CLL-specific ML model was trained to directly predict the malignant B cell fraction from cfRNA. As shown in FIG. 5E, the model predictions correlated well with malignant B cell fractions assessed by flow cytometry (FC) (Spearman: 0.75, p=le-05). As shown in FIG. 5F, the model predictions correlated well with malignant B cell fraction, calculated by multiplying the total deconvolved B cell fraction from cfRNA by the fraction of the dominant BCR clonotype from cfRNA (Spearman: 0.76, p=3e-02).

The model predictions correlated well with cfRNA-derived malignant B cell fraction, calculated by multiplying the total deconvolved B cell fraction by the fraction of the dominant BCR clone (Pearson correlation=0.83, P=0.01). Model outputs divided by the fraction of total B cell populations also correlated with the malignant BCR clonotype fractions (Pearson correlation=0.71, P=0.05), indicating the model's ability to distinguish between malignant and healthy B cells.

### Training

Expression data was obtained for tumor, cell, and types of cell-containing samples listed in Table 7. The expression data included RNA-seq data for the tumor, cell, and cell-containing samples listed in Table 7. The RNA-seq data was obtained from databases including the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10), the BioStudies database (www.ebi.ac.uk/biostudies/), and the European Nucleotide Archive (ENA) database (www.ebi.ac.uk/ena).

The expression data was preprocessed using a plurality of technical and biological quality control techniques. The technical quality control techniques included processing the training data using FastQC (Andrews S. (2010). FastQC: a quality control tool for high throughput sequence data.), FastQ Screen, SNP pileup, HLA matching, RseQC, and DNA contamination assessment. FastQ Screen is described by Wingett, Steven W., and Simon Andrews. (“FastQ Screen: A tool for multi-genome mapping and quality control.” F1000Research 7 (2018).), which is incorporated by reference herein in its entirety. RseQC is described by Wang, Liguo, Shengqin Wang, and Wei Li. (“RSeQC: quality control of RNA-seq experiments.” Bioinformatics 28.16 (2012): 2184-2185.), which is incorporated by reference herein in its entirety. The results of the technical quality control techniques were aggregated into a report using MultiQC. MultiQC is described by Ewels, Philip, et al. (“MultiQC: summarize analysis results for multiple tools and samples in a single report.” Bioinformatics 32.19 (2016): 3047-3048.), which is incorporated by reference herein in its entirety.

After performing the data pre-processing techniques, expression data was renormalized to a set of 18,792 genes, including housekeeping genes. Normalization was performed as in the example described by Abbas-Aghababazadeh, Farnoosh, Qian Li, and Brooke L. Fridley (“Comparison of normalization approaches for gene expression studies completed with high-throughput sequencing.” PloS one 13.10 (2018): e0206312.), which is incorporated by reference herein in its entirety.

The expression data was further filtered to filter out tumor samples having a tumor purity outside the range: [0.6, 1.0], which represents tumor content relative to non-tumor content in the sample.

The expression data was used to generate artificial cfRNA expression profiles representing cfRNA expression data from subjects having CLL with different malignant B cell fractions. All artificial cfRNA expression profiles were generated by: (i) generating a healthy expression profile component, (ii) generating a tumor expression profile component, and (iii) combining the healthy expression profile component and the tumor expression profile component. The tumor expression profile component was generated using a tumor expression profile obtained from the tumor samples listed in Table 7. The healthy expression profile component was generated by combining healthy expression profiles obtained from the non-tumor cell and cell-containing samples listed in Table 7. Different artificial cfRNA expression profiles represent samples with different malignant B cell fractions.

Table 7 also lists the ranges from which the proportions of each cell type, cell-containing sample type, and tumor were selected. The proportions of each cell type and cell-containing sample type were determined according to the techniques described herein including at least with respect to FIG. 1D and Equation 2. The tumor proportion was determined according to techniques described herein including at least with respect to FIG. 1C and Equation 1.

The training data included the artificial cfRNA expression profiles. The artificial cfRNA expression profiles contained different proportions of tumor cells (e.g., malignant B cells). The artificial cfRNA expression profiles were labeled with clinical annotations that indicated malignant B cell fraction. The clinical annotations were used as ground truth.

The training data was used to train a LightGBM decision tree boosting machine learning model to predict the respective proportion (fraction) of malignant B cells relative to total B cells (malignant+healthy B cells) (“malignant B cell fraction”). Specifically, the LightGBM decision tree boosting machine learning model was trained using the artificial cfRNA expression profiles generated for training.

Feature selection was performed on the artificial cfRNA expression training data during optimization of the LightGBM decision tree boosting machine learning model to select a number of feature (e.g., genes). High dropout genes were excluded. Dropout genes included those genes having an expression of 0 in at least 80% of the samples. Genes for which the expression levels of the majority of real samples fell within (e.g., between 0.1 and 0.9 quantiles) the artificial distribution, showing a low KL divergence (30^{th} and 70^{th} percentile), were retained. Feature selection was performed on the artificial cfRNA expression training data to select a number of features (e.g., genes). 150 genes were selected. Feature selection involved generating cross-validated mixes. Each fold excluded one run with healthy plasmas. The number of differentially-expressed genes was reduced for each fold until there were less than 150 genes remaining in the intersection. Gene set enrichment analysis was performed to ensure the biological relevance of the obtained features.

Table 8 lists the parameters of the trained model.

The model was controlled for predicting possible batches in the data instead of the target label. The annotations listed in the description were checked to determine if they explained model prediction better than the target label (malignant B cell fraction) by comparing the AUC curves.

TABLE 7
describes the training data used to train the machine
learning model to predict malignant B cell fraction.

| Sample Class | CLL |
|---|---|
| Number and Types of Tumor | Diagnosis: CLL. |
| Samples Used for Artificial Mixes | Number of samples |
| | 490 sorted cells |
| | 65 tissues |
| Number and Types of Cell, Cell- | Whole Blood: 18,884 |
| Containing, and Tumor Samples | Myeloid Cells: 1,623 |
| Used for Artificial Mixes | Stromal Cells: 1,370 |
| | Lymphoid Cells: 4,046 |
| | Epithelium: 39 |
| | Platelets: 763 |
| | Plasma Cells: 464 |
| Number of Artificial cfRNA | 2,000 |
| Expression Profiles Generated for | |
| Training | |
| Cell, Cell-Containing Sample, and | Whole Blood: [0.0001, 0.6], |
| Tumor Types (and the Lower and | Myeloid Cells: [0.0001, 0.5], |
| Upper Bounds of their Proportions) | Stromal Cells: [0.0001, 0.2], |
| for Which Expression Profiles Were | Platelets: [0.0001, 0.8], |
| Obtained and Used to Generate | Lymphoid Cells: [0.0001, 0.3], |
| Artificial cfRNA Expression Data | Hepatocytes: [0.0001, 0.1], |
| | Epithelium: [0.0001, 0.3], |
| | Tumor: [10⁻⁵, 1], |
| Plasma Proportion Lower and | [0.7, 0.9] |
| Upper Bounds | |

TABLE 8
lists the parameters of the trained CLL-specific ML model.

| Parameter | Value |
|---|---|
| boosting_type | dart |
| feature_fraction | 0.07524359482355654 |
| learning_rate | 0.019753611170716768 |
| max_depth | 4 |
| min_child_samples | 85 |
| n_estimators | 978 |
| num_leaves | 16 |
| reg_alpha | 0.5598418356048347 |
| reg_lambda | 1.0738576432290931e−08 |
| subsample | 0.941831034358922 |
| subsample_freq | 4 |

### CLL Detection

The malignant B cell fraction predicted using the trained CLL-specific model can be used to accurately and reliably determine whether or not a subject has CLL. Specifically, as shown in FIG. 5H, malignant B cell fractions predicted using the trained CLL-specific ML model were used to classify subjects as having or not having CLL. Subjects having a malignant B cell fraction greater than or equal to the limit of detection (LOD) (0.002) were classified as having CLL. As shown in FIG. 5H, healthy subjects (e.g., subjects not having CLL) were accurately classified as not having CLL, while sick subjects (e.g., subjects having CLL) were accurately classified as having CLL. FIG. 5H also shows a limit of quantification (LOQ) of 0.006. FIG. 5I and FIG. J show the validation of the LOD (0.002) and the LOQ (0.006), respectively.

### Example 4—PD1 Status

In this example, a machine learning model was trained to predict the PD-1 status (PDCD1− or PDCD1+) of a subject using cfRNA expression data previously obtained from a blood plasma sample from the subject.

The output of the trained machine learning model can be used for many different applications. For example, the output may be used to identify a treatment that may be used to treat the subject (e.g., by administering the treatment to the subject). A PDCD+status may indicate that the subject will respond to an anti-cancer therapy, such as an immune checkpoint inhibitor (e.g., a PD-1 inhibitor).

### Training Data

Expression data was obtained for tumor, cell, and types of cell-containing samples listed in Table 9. The expression data included RNA-seq data for the tumor, cell, and cell-containing samples listed in Table 9. The RNA-seq data was obtained from databases including the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10) and The Cancer Genome Atlas (TCGA) database (TCGA Research Network: www.cancer.gov/tcga.).

The expression data was preprocessed using a plurality of technical and biological quality control techniques. The technical quality control techniques included processing the training data using FastQC (Andrews S. (2010). FastQC: a quality control tool for high throughput sequence data.), FastQ Screen, SNP pileup, HLA matching, RseQC, and DNA contamination assessment. FastQ Screen is described by Wingett, Steven W., and Simon Andrews. (“FastQ Screen: A tool for multi-genome mapping and quality control.” F1000Research 7 (2018).), which is incorporated by reference herein in its entirety. RseQC is described by Wang, Liguo, Shengqin Wang, and Wei Li. (“RSeQC: quality control of RNA-seq experiments.” Bioinformatics 28.16 (2012): 2184-2185.), which is incorporated by reference herein in its entirety. The results of the technical quality control techniques were aggregated into a report using MultiQC. MultiQC is described by Ewels, Philip, et al. (“MultiQC: summarize analysis results for multiple tools and samples in a single report.” Bioinformatics 32.19 (2016): 3047-3048.), which is incorporated by reference herein in its entirety.

After performing the data pre-processing techniques, expression data was renormalized to a set of 17,454 genes, including housekeeping genes. Example normalization techniques are described by Abbas-Aghababazadeh, Farnoosh, Qian Li, and Brooke L. Fridley (“Comparison of normalization approaches for gene expression studies completed with high-throughput sequencing.” PloS one 13.10 (2018): e0206312.), which is incorporated by reference herein in its entirety.

The expression data was further filtered to filter out tumor samples having a tumor purity outside the range: [0.7, 1.0], which represents tumor content relative to non-tumor content in the sample.

The expression data was used to generate artificial cfRNA expression profiles representing cfRNA expression data from subjects having tumor cells that express PDCD1 (PDCD1+) and artificial cfRNA expression profiles representing cfRNA expression data from subjects having tumor cells that do not express PDCD1 (PDCD1−). All artificial cfRNA expression profiles were generated by: (i) generating a healthy expression profile component, (ii) generating a tumor expression profile component, and (iii) combining the healthy expression profile component and the tumor expression profile component. The tumor expression profile component was generated using a tumor expression profile obtained from the tumor samples listed in Table 9. The healthy expression profile component was generated by combining healthy expression profiles obtained from the non-tumor cell and cell-containing samples listed in Table 9. For the artificial cfRNA expression profiles representing cfRNA expression data from PDCD1+ subjects, the tumor expression profile component was generated using a tumor expression profile obtained from a tumor sample expressing PDCD1. For the artificial cfRNA expression profiles representing cfRNA expression data from PDCD1−, the tumor expression profile component was generated using a tumor expression profile obtained from a tumor sample not expressing PDCD1.

Table 9 also lists the ranges from which the proportions of each cell type, cell-containing sample type, and tumor were selected. The proportions of each cell type and cell-containing sample type were determined according to the techniques described herein including at least with respect to FIG. 1D and Equation 2. The tumor proportion was determined according to techniques described herein including at least with respect to FIG. 1C and Equation 1.

The training data included the artificial cfRNA expression profiles. The artificial cfRNA expression profiles included 5,000 PDCD1+ profiles and 5,000 PDCD1− profiles. The artificial cfRNA expression profiles were labeled with clinical annotations indicating whether or not the expression profile was PDCD1+ or PDCD1−. The clinical annotations were used as ground truth.

The training data was used to train a LightGBM decision tree boosting machine learning model to predict a PD-1 status for the subject given cfRNA expression data obtained for the subject. Specifically, the LightGBM decision tree boosting machine learning model was trained using the artificial cfRNA expression profiles generated for training.

Feature selection was performed on the artificial cfRNA expression training data during optimization of the LightGBM decision tree boosting machine learning model to select a number of feature (e.g., genes). Genes with low expression in tumor (95^{th} percentile expression in tumor is less than 50 TPM) were excluded. Transcripts capturing patient-specific features and ribosomal genes were excluded. High dropout genes were excluded. Dropout genes included those genes having an expression of 0 in at least 80% of the samples. Genes for which the expression levels of the majority of real samples fell within (e.g., between 0.1 and 0.9 quantiles) the artificial distribution, showing a low KL divergence (30^{th} and 70^{th} percentile), were retained. 300 genes were selected. Feature selection involved generating cross-validated mixes. Each fold excluded one run with healthy plasmas. Differentially-expressed genes (DEGs) common for all folds were selected (e.g., less than 2,000 genes for each fold with lowest p-value, p-value <0.05) using the Mann-Whitney test. The DEGs were filtered using real cfRNA samples. Gene set enrichment analysis was performed to ensure the biological relevance of the obtained features. Table 10 lists the genes that were selected.

The model was controlled for predicting possible batches in the data instead of the target label. The annotations listed in the description were checked to determine if they explained model prediction better than the target label (PD-1 status) by comparing the AUC curves.

Table 11 lists the parameters of the trained model.

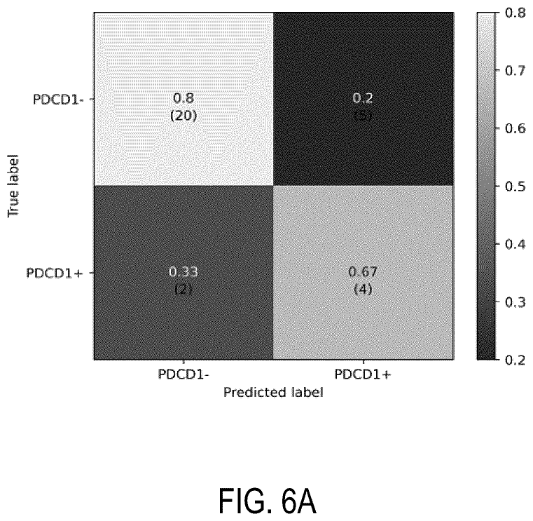

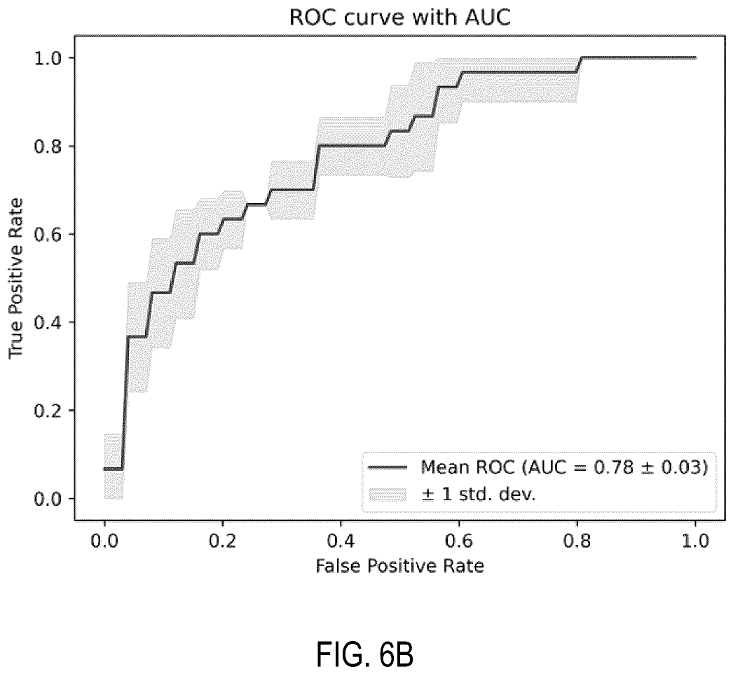

FIG. 6A and FIG. 6B show that the trained machine learning model accurately distinguishes between PDCD1+ and PDCD1− subjects.

TABLE 9
describes the training data used to train the machine learning
model to predict the PD-1 status of the subject.

| Sample Class | PDCD1− | PDCD1+ |
|---|---|---|
| Number and Types of | BRCA: 2,345. | BRCA: 2,345. |
| Tumor Samples Used for | | |
| Artificial Mixes | | |
| Number and Types of Cell, | Whole blood: 18,884 | Whole blood: 18,884 |
| Cell-Containing, and | Myeloid Cells: 1,623 | Myeloid Cells: 1,623 |
| Tumor Samples Used for | Stromal Cells: 1,370 | Stromal Cells: 1,370 |
| Artificial Mixes | Lymphoid Cells: 4,046 | Lymphoid Cells: 4,046 |
| | Epithelium: 39 | Epithelium: 39 |
| | Platelets: 763 | Platelets: 763 |
| Number of Artificial cfRNA | 5,000 | 5,000 |
| Expression Profiles | | |
| Generated for Training | | |
| Cell, Cell-Containing | Whole Blood: [0.0001, 0.6], | Whole Blood: [0.0001, 0.6], |
| Sample, and Tumor Types | Myeloid Cells: [0.0001, 0.5], | Myeloid Cells: [0.0001, 0.5], |
| (and the Lower and Upper | Stromal Cells: [0.0001, 0.2], | Stromal Cells: [0.0001, 0.2], |
| Bounds of their | Platelets: [0.0001, 0.8], | Platelets: [0.0001, 0.8], |
| Proportions) for Which | Lymphoid Cells: [0.0001, | Lymphoid Cells: [0.0001, |
| Expression Profiles Were | 0.3], Epithelium: [0.0001, | 0.3], Epithelium: [0.0001, |
| Obtained and Used to | 0.3], Hepatocytes [0.0001- | 0.3], Hepatocytes [0.0001- |
| Generate Artificial cfRNA | 0.1], Tumor: [0.005, 0.095] | 0.1], Tumor: [0.005, 0.095] |
| Expression Data | | |
| Plasma Proportion Lower | [0.900, 0.950] | [0.900, 0.950] |
| and Upper Bounds | | |

TABLE 10
lists the genes selected during feature selection performed for the PD-1 model.

| | |
|---|---|
| Genes | CXCL13, XIRP1, FDCSP, CPXM1, ADIPOQ, CCL19, CXCL9, IGHG1, |
| | CILP, TUSC5, MMP7, PLA2G2D, TEAD3, AEBP1, IGHG3, KLHDC7B, |
| | EVC, KLK6, C6orf15, GABRP, CPZ, CHRDL1, CCL18, PIF1, FTHL17, |
| | C7, TONSL, HOXA6, OBP2B, PLIN1, RHOXF2, MFAP4, SFRP1, |
| | AMY1C, FREM1, BOC, KLK4, PROL1, SFRP2, STAC2, EMILIN1, |
| | TMEM119, SUSD2, CST1, PRELP, CEP170B, KIAA1755, C3orf36, |
| | C16orf89, CCL21, NGFR, ITIH5, UBD, ITGAE, HR, ARHGEF17, |
| | ADAMTS7, ETV3L, CCL17, PCOLCE, PRRX1, IGKC, SSTR2, |
| | LGALS3BP, COL6A2, HTRA3, TNC, IGSF9, GPD1, FBLN1, |
| | ADAMDEC1, AGRN, HLA-DQB2, MAPK11, C1QTNF1, TUBB2B, |
| | WISP2, C1S, CBX2, ADM5, PRR22, C1QC, FUT3, ADAMTS4, RGMA, |
| | HOXA7, RARRES1, CERCAM, CLSTN3, ITGA7, EFS, COL6A1, C1QB, |
| | CSPG4, NOTCH3, NDUFA4L2, FGFR4, FBLN2, PHGDH, SLC28A3, |
| | AOC3, CAPN6, PLEKHG6, C22orf46, CHIT1, C3, SDC3, PCDHGC3, |
| | PLEKHN1, MFAP2, SERPINH1, IL4I1, CCL13, KCNJ5, MMP12, RGS12, |
| | PTK7, COL9A3, ISLR, DMBX1, CCL22, PAPLN, FZD7, BGN, SLC5A1, |
| | C1R, C2CD2, PDGFRB, KLHL17, EDN2, CILP2, PCDHGB1, FMO1, |
| | SOD3, ADAMTS18, MAP1LC3C, LAMB1, LAMA5, NPR1, COL16A1, |
| | EGFL6, C1orf159, FZD4, MAFIP, RASD2, MMP19, AIFM2, ADAMTS14, |
| | COLEC12, IQGAP3, CHST3, KCNK3, PCDHB4, MRGPRF, ANGPTL2, |
| | COL4A1, SLC1A3, IGDCC4, PLXNA1, SLC19A3, COL15A1, PODN, |
| | ADAMTS2, GEM, VCAM1, ARHGEF25, OSR1, TTLL10, OLFML2B, |
| | PCDHA10, KLB, S1PR2, PKD1, CCR8, DPT, CXCL10, AQP1, RNASE10, |
| | OSMR, LYNX1, APOE, F2RL2, GALNT16, MXRA8, PKDCC, APOL1, |
| | VSTM4, MC1R, WISP1, CD248, CDH3, SCARA3, LOXL1, TYRO3, LEP, |
| | C1QA, SIX5, ESPNL, ST5, UHRF1, SIGLEC1, IGFBP7, SELE, |
| | C1QTNF6, ANGPTL4, GGT5, MMP14, MYO7A, OLFML1, ETV4, SP6, |
| | MCAM, CX3CL1, H6PD, CD276, SMO, ASTN1, ARHGEF19, GTSE1, |
| | BBOX1, RAB42, LMOD1, SLAMF8, STRA6, DCHS1, ENG, PLEKHH2, |
| | TIE1, SORCS2, DERL3, MRAP, PDGFRA, GPX3, HSPG2, ADGRA2, |
| | ADAMTSL2, OAF, LIPG, CLIP3, PNMA2, PQLC2, RAB7B, TCN2, |
| | SLC39A4, ATP13A1, RASL12, TINAGL1, PCDHGA12, C10orf10, |
| | COL5A3, RAD54L, PCDHB14, SLC39A13, ETV7, WNT6, GFPT2, |
| | SCARF2, TNFRSF18, PPARD, PDCD1, WDR86, C1QTNF2, HTR1D, |
| | BMP1, TMEM200C, KCNN4, VASN, CXorf36, TSPAN11, NR1H3, |
| | MOXD1, SERPINF1, C2, EGFLAM, COL18A1, MRC2, LMF2, NLGN2, |
| | NOS3, PALD1, ENPP2, CNTNAP1, PTGIS, SLIT3, LGI4, ARHGAP22, |
| | GPR153, TMEM201, TNFSF15, SSC5D, MBD3, FBLIM1, HHIPL1, |
| | CYGB, CD74, DLL4, GAREML, NOTCH4, FBXO10, EVC2 |

TABLE 11
lists the parameters of the trained PD-1 ML model.

| Parameter | Value |
|---|---|
| boosting_type | dart |
| feature_fraction | 0.07524359482355654 |
| learning_rate | 0.019753611170716768 |
| max_depth | 4 |
| min_child_samples | 85 |
| n_estimators | 978 |
| num_leaves | 16 |
| reg_alpha | 0.5598418356048347 |
| reg_lambda | 1.0738576432290931e−08 |
| subsample | 0.941831034358922 |
| subsample_freq | 4 |

### Example 5

In this example, a machine learning model was trained to predict whether a subject has basal breast cancer using cfRNA expression data previously obtained from a blood plasma sample from the subject.

The output of the trained machine learning model can be used for many different applications. As a first example, the output may be used to confirm and/or prompt the clinical diagnosis of basal breast cancer. For example, when the output of the trained machine learning model indicates that the subject has basal breast cancer, then one or more additional examinations may be performed to confirm and/or measure the extent to which the subject has basal breast cancer. Such examinations may include mammography and/or biopsy. As a second example, the output may be used as a surrogate endpoint in drug clinical trials. For example, when the output of the machine learning model indicates that the subject does not have (or no longer has) basal breast cancer, then therapy administration may be canceled.

### Training Data

Expression data was obtained for tumor, cell, and types of cell-containing samples listed in Table 12. The expression data included RNA-seq data for the tumor, cell, and cell-containing samples listed in Table 12. The RNA-seq data was obtained from databases including the Gene Expression Omnibus (GEO) database (Edgar R, Domrachev M, Lash A E. Gene Expression Omnibus: NCBI gene expression and hybridization array data repository, Nucleic Acids Res. 2002 Jan. 1; 30 (1): 207-10) and The Cancer Genome Atlas (TCGA) database (TCGA Research Network: www.cancer.gov/tcga.).

The expression data was preprocessed using a plurality of technical and biological quality control techniques. The technical quality control techniques included processing the training data using FastQC (Andrews S. (2010). FastQC: a quality control tool for high throughput sequence data.), FastQ Screen, SNP pileup, HLA matching, RseQC, and DNA contamination assessment. FastQ Screen is described by Wingett, Steven W., and Simon Andrews. (“FastQ Screen: A tool for multi-genome mapping and quality control.” F1000Research 7 (2018).), which is incorporated by reference herein in its entirety. RseQC is described by Wang, Liguo, Shengqin Wang, and Wei Li. (“RSeQC: quality control of RNA-seq experiments.” Bioinformatics 28.16 (2012): 2184-2185.), which is incorporated by reference herein in its entirety. The results of the technical quality control techniques were aggregated into a report using MultiQC. MultiQC is described by Ewels, Philip, et al. (“MultiQC: summarize analysis results for multiple tools and samples in a single report.” Bioinformatics 32.19 (2016): 3047-3048.), which is incorporated by reference herein in its entirety.

After performing the data pre-processing techniques, expression data was renormalized to a set of 17,454 genes, including housekeeping genes. Example normalization techniques are described by Abbas-Aghababazadeh, Farnoosh, Qian Li, and Brooke L. Fridley (“Comparison of normalization approaches for gene expression studies completed with high-throughput sequencing.” PloS one 13.10 (2018): e0206312.), which is incorporated by reference herein in its entirety.

The expression data was further filtered to filter out tumor samples having a tumor purity lower than 0.7, which represents tumor content relative to non-tumor content in the sample.

The expression data was used to generate artificial cfRNA expression profiles representing cfRNA expression data from subjects having basal breast cancer and artificial cfRNA expression profiles representing cfRNA expression data from subjects not having basal breast cancer. All artificial cfRNA expression profiles were generated by: (i) generating a healthy expression profile component, (ii) generating a tumor expression profile component, and (iii) combining the healthy expression profile component and the tumor expression profile component. The tumor expression profile component was generated using a tumor expression profile obtained from the tumor samples listed in Table 12. The healthy expression profile component was generated by combining healthy expression profiles obtained from the non-tumor cell and cell-containing samples listed in Table 12. For the artificial cfRNA expression profiles representing cfRNA expression data from subjects having basal breast cancer, the tumor expression profile component was generated using a tumor expression profile from a tumor sample having basal breast cancer. For the artificial cfRNA expression profiles representing cfRNA expression data from subjects not having basal breast cancer, the tumor expression profile component was generated using a tumor expression profile obtained from a tumor sample not having basal breast cancer.

Table 12 also lists the ranges from which the proportions of each cell type, cell-containing sample type, and tumor were selected. The proportions of each cell type and cell-containing sample type were determined according to the techniques described herein including at least with respect to FIG. 1D and Equation 2. The tumor proportion was determined according to techniques described herein including at least with respect to FIG. 1C and Equation 1.

The training data included the artificial cfRNA expression profiles. The artificial cfRNA expression profiles were labeled with clinical annotations that indicated whether or not the expression profile contained tumor (e.g., basal breast cancer) expression data. The clinical annotations were used as ground truth.

The training data was used to train a LightGBM decision tree boosting machine learning model to predict whether or not a subject has basal breast cancer given cfRNA expression data obtained for the subject. Specifically, the LightGBM decision tree boosting machine learning model was trained using the artificial cfRNA expression profiles generated for training.

Feature selection was performed on the artificial cfRNA expression training data during optimization of the LightGBM decision tree boosting machine learning model to select a number of feature (e.g., genes). Genes with low expression in tumor (95^{th} percentile expression in tumor is less than 50 TPM) were excluded. Transcripts capturing patient-specific features and ribosomal genes were excluded. High dropout genes were excluded. Dropout genes included those genes having an expression of 0 in at least 80% of the samples. Genes for which the expression levels of the majority of real samples fell within (e.g., between 0.1 and 0.9 quantiles) the artificial distribution, showing a low KL divergence (30^{th} and 70^{th} percentile), were retained. 150 genes were selected. Feature selection involved generating cross-validated mixes. Each fold excluded one run with healthy plasmas. Differentially-expressed genes (DEGs) common for all folds were selected (e.g., less than 2,000 genes for each fold with lowest p-value, p-value <0.05) using the Mann-Whitney test. The DEGs were filtered using real cfRNA samples. The number of DEGs was reduced for each fold until there were less than 200 genes remaining in the intersection. Gene set enrichment analysis was performed to ensure the biological relevance of the obtained features. Table 13 lists the genes that were selected.

The model was controlled for predicting possible batches in the data instead of the target label. The annotations listed in the description were checked to determine if they explained model prediction better than the target label (basal breast cancer) by comparing the AUC curves.

Table 14 lists the parameters of the trained model.

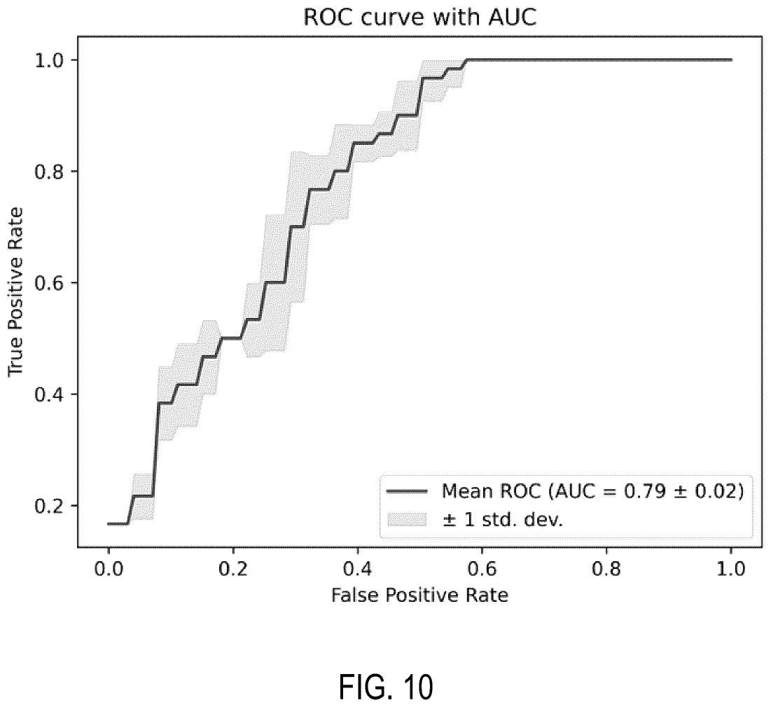

FIG. 10 shows that the trained machine learning model accurately distinguishes between subjects having basal breast cancer and subjects not having basal breast cancer.

TABLE 12
describes the training data used to train the machine learning
model to predict whether a subject has basal breast cancer.

| Sample Class | Basal Breast Cancer | Non-Basal Breast Cancer |
|---|---|---|
| Number and Types of | BRCA: 2,345 | BRCA: 2,345 |
| Tumor Samples Used for | | |
| Artificial Mixes | | |
| Number and Types of Cell, | Whole Blood: 18,884 | Whole Blood: 18,884 |
| Cell-Containing, and | Myeloid Cells: 1,623 | Myeloid Cells: 1,623 |
| Tumor Samples Used for | Stromal Cells: 1,370 | Stromal Cells: 1,370 |
| Artificial Mixes | Lymphoid Cells: 4,046 | Lymphoid Cells: 4,046 |
| | Epithelium: 39 | Epithelium: 39 |
| | Platelets: 763 | Platelets: 763 |
| Number of Artificial cfRNA | 5,000 | 5,000 |
| Expression Profiles | | |
| Generated for Training | | |
| Cell, Cell-Containing | Whole Blood: [0, 0.6], | Whole Blood: [0, 0.6], |
| Sample, and Tumor Types | Myeloid Cells: [0, 0.5], | Myeloid Cells: [0, 0.5], |
| (and the Lower and Upper | Stromal Cells: [0, 0.2], | Stromal Cells: [0, 0.2], |
| Bounds of their | Platelets: [0, 0.8], Lymphoid | Platelets: [0, 0.8], Lymphoid |
| Proportions) for Which | Cells [0, 0.3], Hepatocytes: | Cells [0, 0.3], Hepatocytes: |
| Expression Profiles Were | [0, 0.1], Epithelium: [0, 0.3], | [0, 0.1], and Epithelium: [0, |
| Obtained and Used to | BRCA Tumor [0.05, 0.1] | 0.3], BRCA Tumor [0.05, |
| Generate Artificial cfRNA | | 0.1] |
| Expression Data | | |
| Plasma Proportion Lower | [0.7, 0.9] | [0.7, 0.9] |
| and Upper Bounds | | |

TABLE 13
lists the genes selected during feature selection
performed for the basal breast cancer model.

| | |
|---|---|
| Genes | PPP1R14C, AQP5, KIF2C, NUF2, ZNF521, ASPM, RASD2, CALB2, |
| | FMNL2, FOXM1, PI15, KHDRBS3, CDCA8, TPX2, VCAM1, SFT2D2, |
| | SMO, PEG3, PRR26, RRBP1, CASP14, UQCRHL, KIAA0020, KIFC1, |
| | SPDYE1, KIF20A, BMS1, APOBEC3B, SOCS5, SMG1, SOX4, PDIA6, |
| | CHML, LMO3, PITRM1, SPAG5, POGK, EXOSC6, UHRF1, ANLN, |
| | AURKB, MLLT4, CCNB2, TOP2A, MPDZ, NDC80, KANK4, TRRAP, |
| | PLCE1, LRRN1, FOXP4, LRPPRC, PLK1, TANC1, TAF5L, FAM208B, |
| | NT5DC2, CDK1, YES1, UCK2, NDRG2, TTLL4, AARS2, PAPD7, |
| | NBPF19, SMC5, ATP13A3, RAPH1, MAP2, CDC42BPG, TEAD2, |
| | SAPCD2, RECQL4, PRC1, HELLS, B3GNT5, PVRL1, SLC7A1, TULP3, |
| | TONSL, ITGA9, USP36, ZNF286A, GEN1, DOCK7, MDN1, CLUH, |
| | KIF23, KNTC1, MICALL1, SRPK1, XPOT, KATNAL2, MICAL3, |
| | SLMO1, B3GALNT2, ALMS1, MCM2, ATP2B4, SH3PXD2B, CEP72, |
| | C10orf10, ANKRD36B, NFIX, MEX3C, OTUD6B, MACC1, NCAPD2, |
| | IQCJ-SCHIP1, CCDC14, PRNP, FIGNL1, DOCK4, MSH6, SNAPC3, |
| | KCTD9, MSH2, COA7, FANCA, CCNA2, CSTF3, GPSM2, GMNN, |
| | ARHGAP28, ATAD2, SPC24, NUP205, ZNF460, BTBD3, MALL, CDC7, |
| | QSER1, EZH2, ZNF260, C9orf3, PTAR1, OSBPL3, MYC, CDT1, |
| | TRMT11, GNPTAB, SEPHS1, RIF1, NCAPG2, MCM4, FAM72A, CENPJ, |
| | BICD1, ZNF529 |

TABLE 14
lists the parameters of the trained basal breast cancer ML model.

| Parameter | Value |
|---|---|
| boosting_type | dart |
| feature_fraction | 0.07524359482355654 |
| learning_rate | 0.019753611170716768 |
| max_depth | 4 |
| min_child_samples | 85 |
| n_estimators | 978 |
| num_leaves | 16 |
| reg_alpha | 0.5598418356048347 |
| reg_lambda | 1.0738576432290931e−08 |
| subsample | 0.941831034358922 |
| subsample_freq | 4 |

### Example 6

In this example, machine learning models were trained in accordance with embodiments of the technology described herein for analysis of blood-derived cell-free messenger RNA to infer clinically important features and biomarkers of malignancies.

### Methods

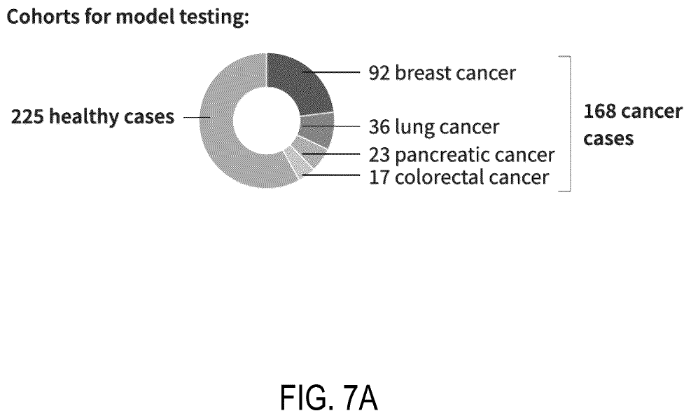

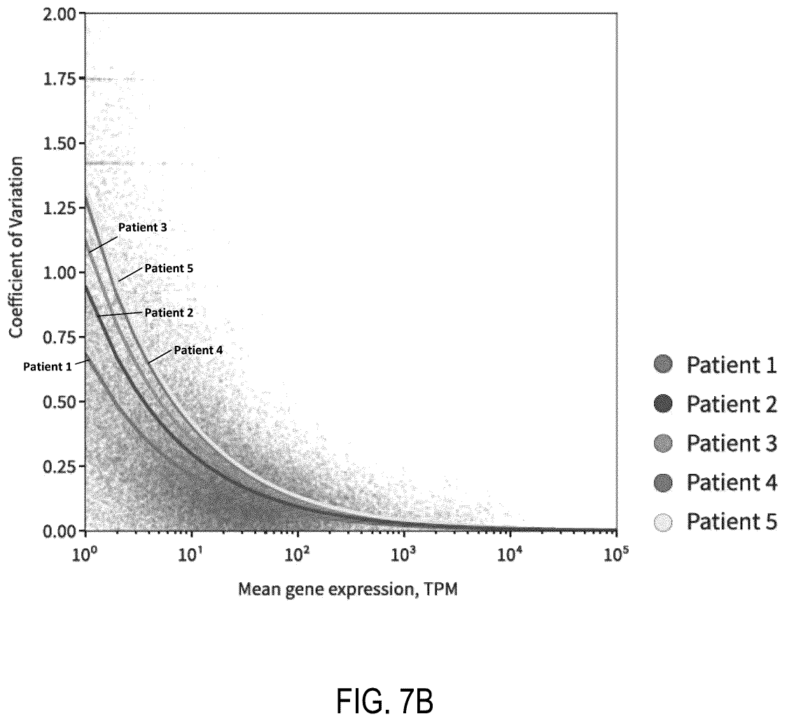

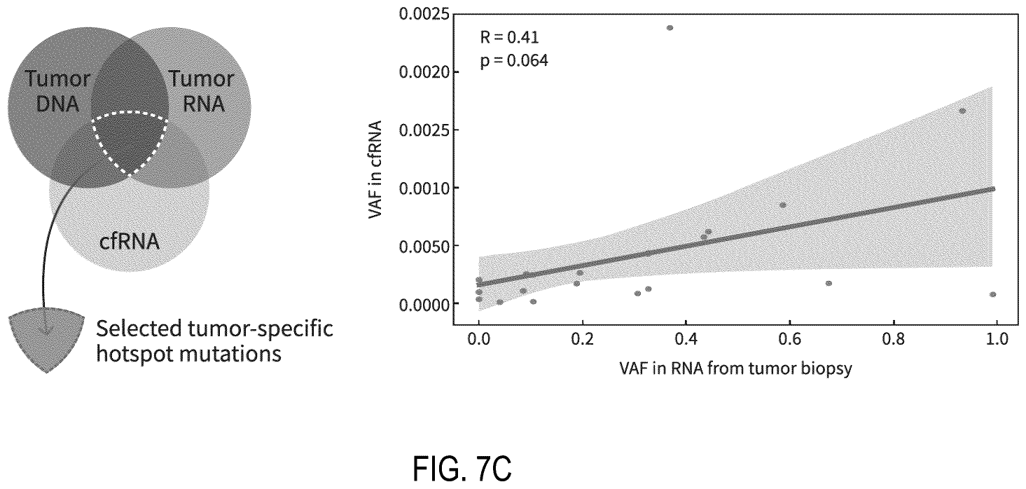

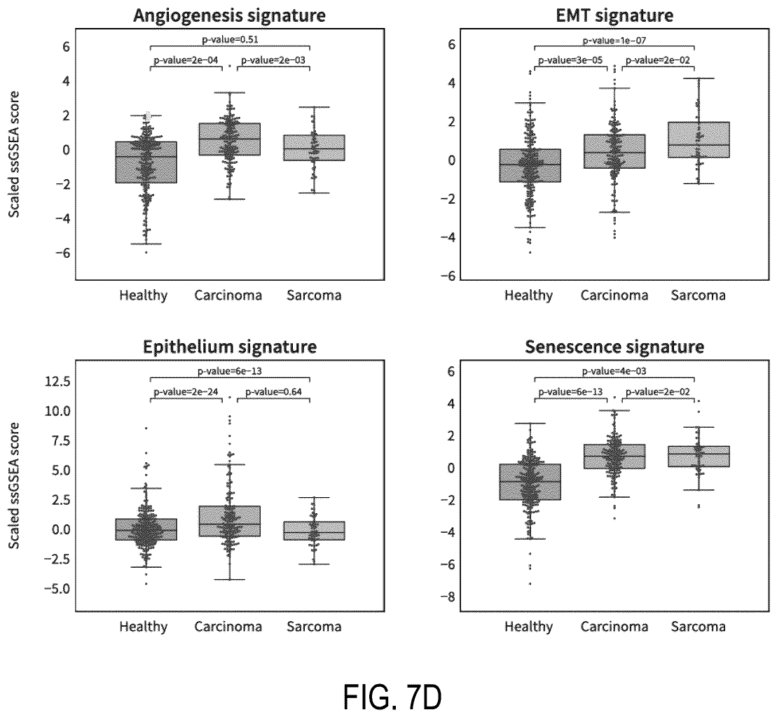

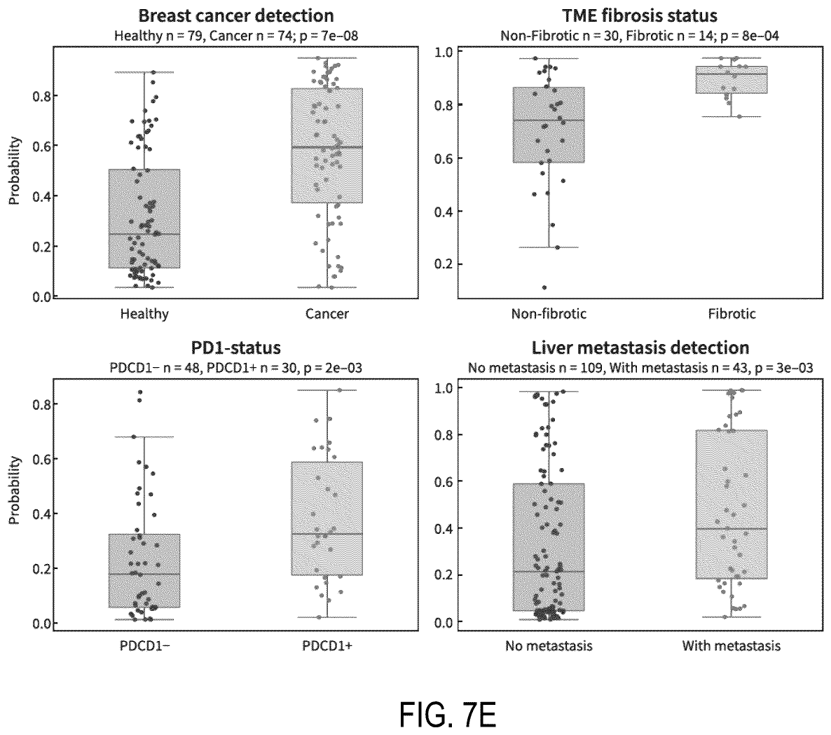

cfRNA was extracted from 4 ml of double-spun plasma of healthy and cancer patients. NGS libraries were prepared according to the Agilent XT HS2 protocol using the VS+UTR exome-wide panel. Pisces 5.2 and samtools mpileup were used to call tumor-specific mutations from cfRNA. Abundance of transcripts from cancer-specific signatures was analyzed using gene set enrichment analysis (GSEA) and single-sample GSEA. ML decision tree-based models were trained on artificial transcriptomes generated from open source bulk RNA-seq data from cancer cells, tissues, and sorted cells collected across the GEO database. Model testing was performed on real cfRNA transcriptomes (n=255 healthy, n=168 cancer cases). FIG. 7A shows the cohorts for model testing.

Highly Reproducible Detection of cfRNA Transcripts

FIG. 7B shows that the detection of cfRNA transcripts is highly reproducible. Robust cfRNA extraction and sequencing protocols ensure reproducible profiling of cfRNA transcriptomes. The plot in FIG. 7B shows the mean cfRNA levels (x axis) versus the coefficients of variation (y axis) for 5 healthy individuals with 6 technical repeats per individual. Transcripts are depicted as dots.

cfRNA Transcriptomes Contain Tumor-Derived Transcripts

The cfRNA profiles from 18 cancer patients contained cfRNA transcripts with tumor-specific hotspot mutations that matched whole exome sequencing (WES) data, demonstrating the presence of tumor signals in cfRNA. As shown in FIG. 7C, a moderate positive correlation was observed between tumor RNA and cfRNA variant allele frequencies (VAFs), with a correlation coefficient of R=0.41 and a p-value of 0.064, indicating a trend towards statistical significance.

cfRNA Transcriptomes Reflect Complex Tumor- and Disease-Specific Signals

FIG. 7D shows that the results of ssGSEA analysis demonstrate tumor-related signatures are enriched in cfRNA transcriptomes from sarcoma (n=52) and carcinoma (n=155) patients compared to healthy patients (n=255), indicating the presence of complex tumor-derived transcriptomic signals.

ML Models Infer Clinically Important Tumor Characteristics from cfRNA Transcriptomes

FIG. 7E shows that ML models trained on artificial cfRNA transcriptomes accurately detect breast cancer status, assess fibrosis in the tumor microenvironment, predict PDCD1 (PD-1) status, and identify liver damage caused by metastasis.

### Example 7

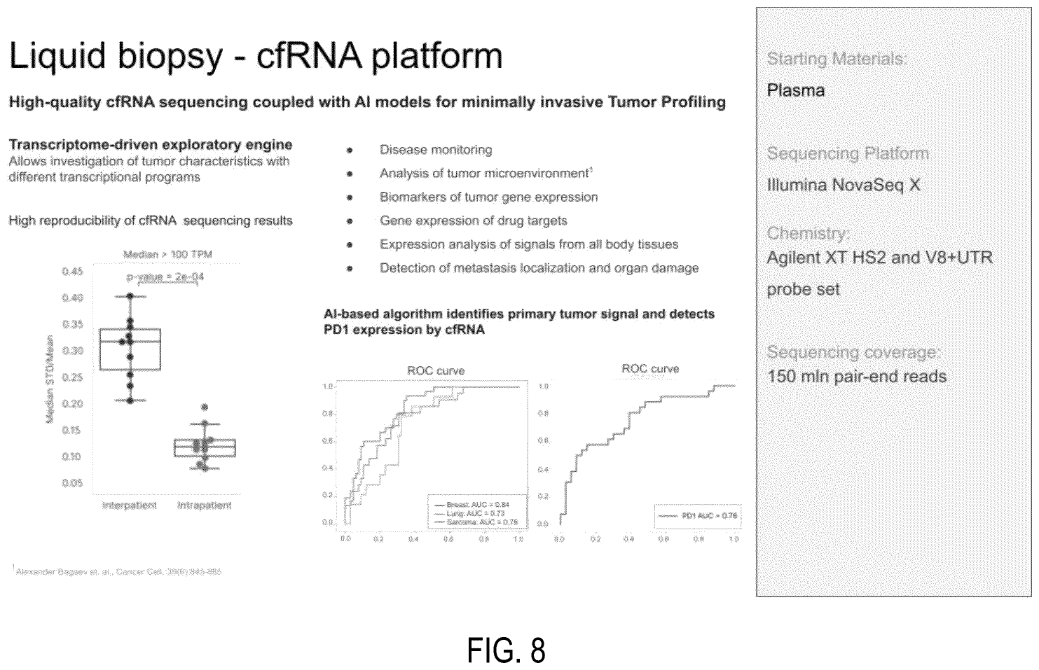

FIG. 8 is an example showing results indicating high reproducibility of cfRNA sequencing results and results of processing artificial cfRNA expression data using machine learning models trained to identify primary tumor signals and detect PD1 expression, according to some embodiments of the technology described herein.

The results were obtained for plasma samples obtained for subjects. The plasma samples were sequenced using Illumina NovaSeq X. Sequencing chemistry included Agilent XT HS2 and the V8+UTR probe kit. The sequencing coverage was 150 million pair-end reads.

As shown in FIG. 8, cfRNA expression data can be used for minimally invasive tumor profiling including disease monitoring, analysis of tumor microenvironment, biomarkers of tumor gene expression, gene expression of drug targets, expression analysis of signals from all body tissues, and/or detection of metastasis localization and organ damage.

### Biological Samples

Any of the methods, systems, or other claimed elements may use or be used to analyze a biological sample from a subject. In some embodiments, a biological sample is obtained from a subject having, suspected of having cancer, or at risk of having cancer. A biological sample may be any type of biological sample including, for example, a biological sample of a bodily fluid (e.g., blood, urine or cerebrospinal fluid), one or more cells (e.g., from a scraping or brushing such as a cheek swab or tracheal brushing), a piece of tissue (cheek tissue, muscle tissue, lung tissue, heart tissue, brain tissue, or skin tissue), or some or all of an organ (e.g., brain, lung, liver, bladder, kidney, pancreas, intestines, or muscle), or other types of biological samples (e.g., feces or hair).

In some embodiments, the biological sample is a sample of a tumor from a subject. In some embodiments, the biological sample is a sample of blood from a subject. In some embodiments, the biological sample is a sample of tissue from a subject.

A sample of a tumor, in some embodiments, refers to a sample comprising cells from a tumor. In some embodiments, the sample of the tumor comprises cells from a benign tumor, e.g., non-cancerous cells. In some embodiments, the sample of the tumor comprises cells from a premalignant tumor, e.g., precancerous cells. In some embodiments, the sample of the tumor comprises cells from a malignant tumor, e.g., cancerous cells. In some embodiments, the sample of tumor can include a mixture of cancerous, non-cancerous, and/or precancerous cells.

Examples of tumors include, but are not limited to, adenomas, fibromas, hemangiomas, lipomas, cervical dysplasia, metaplasia of the lung, leukoplakia, carcinoma, sarcoma, germ cell tumors, melanomas, mesotheliomas, gliomas, and blastoma.

A sample of blood, in some embodiments, refers to a sample comprising cells, e.g., cells from a blood sample. In some embodiments, the sample of blood comprises non-cancerous cells. In some embodiments, the sample of blood comprises precancerous cells. In some embodiments, the sample of blood comprises cancerous cells. In some embodiments, the sample of blood comprises blood cells. In some embodiments, the sample of blood comprises red blood cells. In some embodiments, the sample of blood comprises white blood cells. In some embodiments, the sample of blood comprises platelets. Examples of cancerous blood cells include, but are not limited to, leukemia, lymphoma, and myeloma. In some embodiments, a sample of blood is collected to obtain the cell-free nucleic acid (e.g., cell-free DNA) in the blood.

A sample of blood may be a sample of whole blood or a sample of fractionated blood. In some embodiments, the sample of blood comprises whole blood. In some embodiments, the sample of blood comprises fractionated blood. In some embodiments, the sample of blood comprises buffy coat. In some embodiments, the sample of blood comprises serum. In some embodiments, the sample of blood comprises plasma. In some embodiments, the sample of blood comprises a blood clot.

A sample of tissue, in some embodiments, refers to a sample comprising cells from a tissue. In some embodiments, the sample of the tumor comprises non-cancerous cells from a tissue. In some embodiments, the sample of the tumor comprises precancerous cells from a tissue. In some embodiments, the sample of the tumor comprises cancerous tissue. In some embodiments, the sample can comprise cancerous, precancerous, or non-cancerous cells.

Methods of the present disclosure encompass a variety of tissue including organ tissue or non-organ tissue, including but not limited to, muscle tissue, brain tissue, lung tissue, liver tissue, epithelial tissue, connective tissue, and nervous tissue. In some embodiments, the tissue may be normal tissue, or it may be diseased tissue, or it may be tissue suspected of being diseased. In some embodiments, the tissue may be sectioned tissue or whole intact tissue. In some embodiments, the tissue may be animal tissue or human tissue. Animal tissue includes, but is not limited to, tissues obtained from rodents (e.g., rats or mice), primates (e.g., monkeys), dogs, cats, and farm animals.

The biological sample may be from any source in the subject's body including, but not limited to, any fluid [such as blood (e.g., whole blood, blood serum, or blood plasma), saliva, tears, synovial fluid, cerebrospinal fluid, pleural fluid, pericardial fluid, ascitic fluid, and/or urine], hair, skin (including portions of the epidermis, dermis, and/or hypodermis), oropharynx, laryngopharynx, esophagus, stomach, bronchus, salivary gland, tongue, oral cavity, nasal cavity, vaginal cavity, anal cavity, bone, bone marrow, brain, thymus, spleen, small intestine, appendix, colon, rectum, anus, liver, biliary tract, pancreas, kidney, ureter, bladder, urethra, uterus, vagina, vulva, ovary, cervix, scrotum, penis, prostate, testicle, seminal vesicles, and/or any type of tissue (e.g., muscle tissue, epithelial tissue, connective tissue, or nervous tissue).

Any of the biological samples described herein may be obtained or may have been obtained from the subject using any known technique. See, for example, the following publications on collecting, processing, and storing biological samples, each of which are incorporated herein in its entirety: Biospecimens and biorepositories: from afterthought to science by Vaught et al. (Cancer Epidemiol Biomarkers Prev. 2012 February; 21 (2): 253-5), and Biological sample collection, processing, storage and information management by Vaught and Henderson (IARC Sci Publ. 2011; (163): 23-42).

In some embodiments, the biological sample may have been obtained from a surgical procedure (e.g., laparoscopic surgery, microscopically controlled surgery, or endoscopy), bone marrow biopsy, punch biopsy, endoscopic biopsy, or needle biopsy (e.g., a fine-needle aspiration, core needle biopsy, vacuum-assisted biopsy, or image-guided biopsy).

In some embodiments, one or more than one cell (a cell biological sample) may have been obtained from a subject using a scrape or brush method. The cell biological sample may be obtained from any area in or from the body of a subject including, for example, from one or more of the following areas: the cervix, esophagus, stomach, bronchus, or oral cavity. In some embodiments, one or more than one piece of tissue (e.g., a tissue biopsy) from a subject may be used. In certain embodiments, the tissue biopsy may comprise one or more than one (e.g., 2, 3, 4, 5, 6, 7, 8, 9, 10, or more than 10) biological samples from one or more tumors or tissues known or suspected of having cancerous cells.

Any of the biological samples from a subject described herein may be stored using any method that preserves stability of the biological sample. In some embodiments, preserving the stability of the biological sample means inhibiting components (e.g., DNA, RNA, protein, or tissue structure or morphology) of the biological sample from degrading until they are measured so that when measured, the measurements represent the state of the sample at the time of obtaining it from the subject. In some embodiments, a biological sample is stored in a composition that is able to penetrate the same and protect components (e.g., DNA, RNA, protein, or tissue structure or morphology) of the biological sample from degrading. As used herein, degradation is the transformation of a component from one from to another such that the first form is no longer detected at the same level as before degradation.

In some embodiments, a biological sample (e.g., tissue sample) is fixed. As used herein, a “fixed” sample relates to a sample that has been treated with one or more agents or processes in order to prevent or reduce decay or degradation, such as autolysis or putrefaction, of the sample. Examples of fixative processes include but are not limited to heat fixation, immersion fixation, and perfusion. In some embodiments a fixed sample is treated with one or more fixative agents. Examples of fixative agents include but are not limited to cross-linking agents (e.g., aldehydes, such as formaldehyde, formalin, glutaraldehyde, etc.), precipitating agents (e.g., alcohols, such as ethanol, methanol, acetone, xylene, etc.), mercurials (e.g., B-5, Zenker's fixative, etc.), picrates, and Hepes-glutamic acid buffer-mediated organic solvent protection effect (HOPE) fixatuve. In some embodiments, a biological sample (e.g., tissue sample) is treated with a cross-linking agent. In some embodiments, the cross-linking agent comprises formalin. In some embodiments, a formalin-fixed biological sample is embedded in a solid substrate, for example paraffin wax. In some embodiments, the biological sample is a formalin-fixed paraffin-embedded (FFPE) sample. Methods of preparing FFPE samples are known, for example as described by Li et al. JCO Precis Oncol. 2018; 2: PO.17.00091.

In some embodiments, the biological sample is stored using cryopreservation. Examples of cryopreservation include, but are not limited to, step-down freezing, blast freezing, direct plunge freezing, snap freezing, slow freezing using a programmable freezer, and vitrification. In some embodiments, the biological sample is stored using lyophilization. In some embodiments, a biological sample is placed into a container that already contains a preservant (e.g., RNALater to preserve RNA) and then frozen (e.g., by snap-freezing), after the collection of the biological sample from the subject. In some embodiments, such storage in frozen state is done immediately after collection of the biological sample. In some embodiments, a biological sample may be kept at either room temperature or 4° C. for some time (e.g., up to an hour, up to 8 h, or up to 1 day, or a few days) in a preservant or in a buffer without a preservant, before being frozen.

Examples of preservants include formalin solutions, formaldehyde solutions, RNALater or other equivalent solutions, TriZol or other equivalent solutions, DNA/RNA Shield or equivalent solutions, EDTA (e.g., Buffer AE (10 mM Tris·Cl; 0.5 mM EDTA, pH 9.0)) and other coagulants, and Acids Citrate Dextronse (e.g., for blood specimens).

In some embodiments, special containers may be used for collecting and/or storing a biological sample. For example, a vacutainer may be used to store blood. In some embodiments, a vacutainer may comprise a preservant (e.g., a coagulant, or an anticoagulant). In some embodiments, a container in which a biological sample is preserved may be contained in a secondary container, for the purpose of better preservation, or for the purpose of avoid contamination.

Any of the biological samples from a subject described herein may be stored under any condition that preserves stability of the biological sample. In some embodiments, the biological sample is stored at a temperature that preserves stability of the biological sample. In some embodiments, the sample is stored at room temperature (e.g., 25° C.). In some embodiments, the sample is stored under refrigeration (e.g., 4° C.). In some embodiments, the sample is stored under freezing conditions (e.g., −20° C.). In some embodiments, the sample is stored under ultralow temperature conditions (e.g., −50° C. to −800° C.). In some embodiments, the sample is stored under liquid nitrogen (e.g., −1700° C.). In some embodiments, a biological sample is stored at −60° C. to −80° C. (e.g., −70° C.) for up to 5 years (e.g., up to 1 month, up to 2 months, up to 3 months, up to 4 months, up to 5 months, up to 6 months, up to 7 months, up to 8 months, up to 9 months, up to 10 months, up to 11 months, up to 1 year, up to 2 years, up to 3 years, up to 4 years, or up to 5 years). In some embodiments, a biological sample is stored as described by any of the methods described herein for up to 20 years (e.g., up to 5 years, up to 10 years, up to 15 years, or up to 20 years).

Methods of the present disclosure encompass obtaining one or more biological samples from a subject for analysis. In some embodiments, one biological sample is collected from a subject for analysis. In some embodiments, more than one (e.g., 2, 3, 4, 5, 6, 7, 8, 9, 10, 11, 12, 13, 14, 15, 16, 17, 18, 19, 20, or more) biological samples are collected from a subject for analysis. In some embodiments, one biological sample from a subject will be analyzed. In some embodiments, more than one (e.g., 2, 3, 4, 5, 6, 7, 8, 9, 10, 11, 12, 13, 14, 15, 16, 17, 18, 19, 20, or more) biological samples may be analyzed. If more than one biological sample from a subject is analyzed, the biological samples may be procured at the same time (e.g., more than one biological sample may be taken in the same procedure), or the biological samples may be taken at different times (e.g., during a different procedure including a procedure 1, 2, 3, 4, 5, 6, 7, 8, 9, 10 days; 1, 2, 3, 4, 5, 6, 7, 8, 9, 10 weeks; 1, 2, 3, 4, 5, 6, 7, 8, 9, 10 months, 1, 2, 3, 4, 5, 6, 7, 8, 9, 10 years, or 1, 2, 3, 4, 5, 6, 7, 8, 9, 10 decades after a first procedure).

A second or subsequent biological sample may be taken or obtained from the same region (e.g., from the same tumor or area of tissue) or a different region (including, e.g., a different tumor). A second or subsequent biological sample may be taken or obtained from the subject after one or more treatments and may be taken from the same region or a different region. For example, the second or subsequent biological sample may be useful in determining whether the cancer in each biological sample has different characteristics (e.g., in the case of biological samples taken from two physically separate tumors in a patient) or whether the cancer has responded to one or more treatments (e.g., in the case of two or more biological samples from the same tumor or different tumors prior to and subsequent to a treatment). In some embodiments, each of the at least one biological sample is a bodily fluid sample, a cell sample, or a tissue biopsy sample.

In some embodiments, one or more biological specimens are combined (e.g., placed in the same container for preservation) before further processing. For example, a first sample of a first tumor obtained from a subject may be combined with a second sample of a second tumor from the subject, wherein the first and second tumors may or may not be the same tumor. In some embodiments, a first tumor and a second tumor are similar but not the same (e.g., two tumors in the brain of a subject). In some embodiments, a first biological sample and a second biological sample from a subject are sample of different types of tumors (e.g., a tumor in muscle tissue and brain tissue).

In some embodiments, a sample from which RNA is extracted (e.g., a sample of tumor, or a blood sample) is sufficiently large such that at least 2 μg (e.g., at least 2 μg, at least 2.5 μg, at least 3 μg, at least 3.5 μg or more) of RNA can be extracted from it. In some embodiments, the sample from which RNA is extracted can be peripheral blood mononuclear cells (PBMCs). In some embodiments, the sample from which RNA and/or DNA is extracted can be any type of cell suspension. In some embodiments, a sample from which RNA is extracted (e.g., a sample of tumor, or a blood sample) is sufficiently large such that at least 1.8 μg RNA can be extracted from it. In some embodiments, at least 50 mg (e.g., at least 1 mg, at least 2 mg, at least 3 mg, at least 4 mg, at least 5 mg, at least 10 mg, at least 12 mg, at least 15 mg, at least 18 mg, at least 20 mg, at least 22 mg, at least 25 mg, at least 30 mg, at least 35 mg, at least 40 mg, at least 45 mg, or at least 50 mg) of tissue sample is collected from which RNA is extracted. In some embodiments, at least 20 mg of tissue sample is collected from which RNA is extracted. In some embodiments, at least 30 mg of tissue sample is collected. In some embodiments, at least 10-50 mg (e.g., 10-50 mg, 10-15 mg, 10-30 mg, 10-40 mg, 20-30 mg, 20-40 mg, 20-50 mg, or 30-50 mg) of tissue sample is collected from which RNA is extracted. In some embodiments, at least 30 mg of tissue sample is collected. In some embodiments, at least 20-30 mg of tissue sample is collected from which RNA is extracted. In some embodiments, a sample from which RNA is extracted (e.g., a sample of tumor, or a blood sample) is sufficiently large such that at least 0.2 μg (e.g., at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it. In some embodiments, a sample from which RNA is extracted (e.g., a sample of tumor, or a blood sample) is sufficiently large such that at least 0.1 μg (e.g., at least 100 ng, at least 200 ng, at least 300 ng, at least 400 ng, at least 500 ng, at least 600 ng, at least 700 ng, at least 800 ng, at least 900 ng, at least 1 μg, at least 1.1 μg, at least 1.2 μg, at least 1.3 μg, at least 1.4 μg, at least 1.5 μg, at least 1.6 μg, at least 1.7 μg, at least 1.8 μg, at least 1.9 μg, or at least 2 μg) of RNA can be extracted from it.

### Expression Data

Aspects of the disclosure relate to methods of using a trained machine learning model to predict a characteristic of a subject based on cell-free RNA (cfRNA) expression data previously-obtained from a blood sample from the subject. Additional or alternative aspects of the disclosure relate to training a machine learning model to predict a characteristic of a subject using data generated from RNA expression data.

The cfRNA and RNA expression data (“expression data”) used in methods described herein typically is derived from sequencing data obtained from the biological sample.

The sequencing data may be obtained from the biological sample using any suitable sequencing technique and/or apparatus (e.g., sequencing platform 102 shown in FIG. 1A and FIG. 1F). In some embodiments, the sequencing apparatus used to sequence the biological sample may be selected from any suitable sequencing apparatus known in the art including, but not limited to, Illumina™, SOLid™, Ion Torrent™, PacBio™, a nanopore-based sequencing apparatus, a Sanger sequencing apparatus, or a 454™ sequencing apparatus. In some embodiments, sequencing apparatus used to sequence the biological sample is an Illumina sequencing (e.g., NovaSeq™, NextSeq™, HiSeq™, MiSeq™, or MiniSeq™) apparatus. After the sequencing data is obtained, it is processed in order to obtain the expression data. Expression data may be acquired using any method known in the art including, but not limited to whole transcriptome sequencing, whole exome sequencing, total RNA sequencing, mRNA sequencing, targeted RNA sequencing, RNA exome capture sequencing, next generation sequencing, and/or deep RNA sequencing. In some embodiments, expression data may be obtained using a microarray assay.

In some embodiments, the sequencing data is processed to produce expression data. In some embodiments, sequence data is processed by one or more bioinformatics methods or software tools, for example RNA sequence quantification tools (e.g., Kallisto) and genome annotation tools (e.g., Gencode v23), in order to produce expression data. The Kallisto software is described in Nicolas L Bray, Harold Pimentel, Páll Melsted and Lior Pachter, Near-optimal probabilistic RNA-seq quantification, Nature Biotechnology 34, 525-527 (2016), doi: 10.1038/nbt.3519, which is incorporated by reference in its entirety herein.

In some embodiments, microarray expression data is processed using a bioinformatics R package, such as “affy” or “limma,” in order to produce expression data. The “affy” software is described in Bioinformatics. 2004 Feb. 12; 20 (3): 307-15. doi: 10.1093/bioinformatics/btg405. “affy—analysis of Affymetrix GeneChip data at the probe level” by Laurent Gautier 1, Leslie Cope, Benjamin M Bolstad, Rafael A Irizarry PMID: 14960456 DOI: 10.1093/bioinformatics/btg405, which is incorporated by reference herein in its entirety. The “limma” software is described in Ritchie M E, Phipson B, Wu D, Hu Y, Law C W, Shi W, Smyth G K “limma powers differential expression analyses for RNA-sequencing and microarray studies.” Nucleic Acids Res. 2015 Apr. 20; 43 (7): e47. 20. doi.org/10.1093/nar/gkv007PMID: 25605792, PMCID: PMC4402510, which is incorporated by reference herein its entirety.

In some embodiments, sequencing data and/or expression data comprises more than 5 kilobases (kb). In some embodiments, the size of the obtained data is at least 10 kb. In some embodiments, the size of the obtained sequencing data is at least 100 kb. In some embodiments, the size of the obtained sequencing data is at least 500 kb. In some embodiments, the size of the obtained sequencing data is at least 1 megabase (Mb). In some embodiments, the size of the obtained sequencing data is at least 10 Mb. In some embodiments, the size of the obtained sequencing data is at least 100 Mb. In some embodiments, the size of the obtained sequencing data is at least 500 Mb. In some embodiments, the size of the obtained sequencing data is at least 1 gigabase (Gb). In some embodiments, the size of the obtained sequencing data is at least 10 Gb. In some embodiments, the size of the obtained RNA sequencing data is at least 100 Gb. In some embodiments, the size of the obtained sequencing data is at least 500 Gb.

In some embodiments, the expression data is acquired through bulk RNA sequencing. Bulk RNA sequencing may include obtaining expression levels for each gene across RNA extracted from a large population of input cells (e.g., a mixture of different cell types.) In some embodiments, the expression data is acquired through single cell sequencing (e.g., scRNA-seq). Single cell sequencing may include sequencing individual cells.

In some embodiments, bulk sequencing data comprises at least 1 million reads, at least 5 million reads, at least 10 million reads, at least 20 million reads, at least 50 million reads, or at least 100 million reads. In some embodiments, bulk sequencing data comprises between 1 million reads and 5 million reads, 3 million reads and 10 million reads, 5 million reads and 20 million reads, 10 million reads and 50 million reads, 30 million reads and 100 million reads, or 1 million reads and 100 million reads (or any number of reads including, and between).

In some embodiments, the expression data comprises next-generation sequencing (NGS) data. In some embodiments, the expression data comprises microarray data.

Expression data (e.g., indicating expression levels) for a plurality of genes may be used for any of the methods or compositions described herein. The number of genes which may be examined may be up to and inclusive of all the genes of the subject. In some embodiments, expression levels may be determined for all of the genes of a subject. As a non-limiting example, In some embodiments, expression levels may be obtained for at least 25 genes, at least 50 genes, at least 75 genes, at least 100 genes, at least 150 genes, at least 200 genes, at least 250 genes, at least 500 genes, at least 1,000 genes, at least 1,500 genes, at least 2,000 genes, at least 2,500 genes, at least 3,000 genes, at least 3,500 genes, at least 4,000 genes, at least 4,500 genes, at least 5,000 genes, at least 6000 genes, at least 7,000 genes, at least 8,000 genes, at least 9,000 genes, at least 10,000 genes, at least 15,000 genes, at least 20,000 genes, or at least any other suitable number of genes, as aspects of the technology described herein are not limited in this respect. In some embodiments, expression levels may be obtained for at most 25 genes, at most 50 genes, at most 75 genes, at most 100 genes, at most 150 genes, at most 200 genes, at most 250 genes, at most 500 genes, at most 1,000 genes, at most 1,500 genes, at most 2,000 genes, at most 2,500 genes, at most 3,000 genes, at most 3,500 genes, at most 4,000 genes, at most 4,500 genes, at most 5,000 genes, at most 6000 genes, at most 7,000 genes, at most 8,000 genes, at most 9,000 genes, at most 10,000 genes, at most 15,000 genes, at most 20,000 genes, or at most any other suitable number of genes, as aspects of the technology described herein are not limited in this respect. It should be appreciated that any of the above-listed upper bounds may be coupled with any of the above-listed lower bounds. In some embodiments, As another set of non-limiting examples, the expression data may include, for each set of genes listed in Table 1, expression data for at least some (e.g., all) of the genes included in the particular set of genes.

In some embodiments, expression data is obtained by accessing the expression data from at least one computer storage medium on which the expression data is stored. Additionally or alternatively, in some embodiments, expression data may be received from one or more sources via a communication network of any suitable type. For example, in some embodiment, the expression data may be received from a server (e.g., a SFTP server, or Illumina BaseSpace).

The expression data obtained may be in any suitable format, as aspects of the technology described herein are not limited in this respect. For example, in some embodiments, the expression data may be obtained in a text-based file (e.g., in a FASTQ, FASTA, BAM, or SAM format). In some embodiments, a file in which sequencing data is stored may contains quality scores of the sequencing data. In some embodiments, a file in which sequencing data is stored may contain sequence identifier information.

Expression data, in some embodiments, includes gene expression levels. Gene expression levels may be detected by detecting a product of gene expression such as mRNA and/or protein. In some embodiments, gene expression levels are determined by detecting a level of a mRNA in a sample. As used herein, the terms “determining” or “detecting” may include assessing the presence, absence, quantity and/or amount (which can be an effective amount) of a substance within a sample, including the derivation of qualitative or quantitative concentration levels of such substances, or otherwise evaluating the values and/or categorization of such substances in a sample from a subject.

In some embodiments, sequencing data is processed to obtain expression data from the sequencing data. For example, the sequencing data may be processed using any suitable computing device or devices, as aspects of the technology described herein are not limited in this respect. For example, the processing may be performed by a computing device part of a sequencing apparatus. In other embodiments, the processing may be performed by one or more computing devices external to the sequencing apparatus.

In some embodiments, processing the sequencing data to obtain RNA expression data from the sequencing data includes normalizing the sequencing data to transcripts per kilobase million (TPM) units. The normalization may be performed using any suitable software and in any suitable way. For example, in some embodiments, TPM normalization may be performed according to the techniques described in Wagner et al. (Theory Biosci. (2012) 131:281-285), which is incorporated by reference herein in its entirety. In some embodiments, the TPM normalization may be performed using a software package, such as, for example, the germa package. Aspects of the germa package are described in Wu J, Gentry RIwcfJMJ (2021). “gcrma: Background Adjustment Using Sequence Information. R package version 2.66.0.,” which is incorporated by reference in its entirety herein. In some embodiments, expression level in TPM units for a particular gene may be calculated according to the following formula:

$$A \cdot \frac{1}{\sum ( A )} \cdot {10}^{6} \\ where A = \frac{total reads mapped to gene \cdot {10}^{3}}{gene length in bp}$$

Next, in some embodiments, the expression levels in TPM units may be log transformed.

In some embodiments, the expression levels may not be normalized to transcripts per million units and may, instead, be converted to another type of unit (e.g., reads per kilobase million (RPKM) or fragments per kilobase million (FPKM) or any other suitable unit). Additionally or alternatively, in some embodiments, the log transformation may be omitted. Instead, no transformation may be applied in some embodiments, or one or more other transformations may be applied in lieu of the log transformation.

In some embodiments, the expression data is obtained by processing sequence data generated by a sequencing protocol (e.g., the series of nucleotides in a nucleic acid molecule identified by next-generation sequencing, sanger sequencing, etc.) as well as information contained therein (e.g., information indicative of source, tissue type, etc.) which may also be considered information that can be inferred or determined from the sequence data. In some embodiments, expression data obtained by processing the sequence data can include information included in a FASTA file, a description and/or quality scores included in a FASTQ file, an aligned position included in a BAM file, and/or any other suitable information obtained from any suitable file.

In some embodiments, enrichment scores for genes in one or more sets of genes are determined. In some embodiments, an enrichment score is generated using a gene set enrichment analysis (GSEA) technique, using expression levels of at least some genes in a set of genes. In some embodiments, using a GSEA technique comprises using single-sample GSEA. Aspects of single sample GSEA (ssGSEA) are described in Barbie et al. Nature. 2009 Nov. 5; 462 (7269): 108-112, the entire contents of which are incorporated by reference herein. In some embodiments, ssGSEA is performed according to the following formula:

$$ssGSEA score = \frac{{{\sum }_{i}}^{N} {r}_{i}^{1.25}}{{{\sum }_{i}}^{N} {r}_{i}^{0.25}} - \frac{( M - N + 1 )}{2}$$

where rᵢ represents the rank of the ith gene in expression matrix, where N represents the number of genes in the gene set, and where M represents total number of genes in expression matrix. Additional, suitable techniques of performing GSEA are known in the art and are contemplated for use in the methods described herein without limitation. In some embodiments, an enrichment score is calculated by performing ssGSEA on expression data from a plurality of subjects, for example expression data from one or more cohorts of subjects, such as TCGA, Metabric, FUSCCTNBC, GSE103091, GSE106977, GSE21653, GSE25066, GSE41998, GSE47994, GSE81538, GSE96058, etc., in order to produce a plurality of enrichment scores.

### Subjects

Aspects of this disclosure relate to a biological sample that has been obtained from a subject. In some embodiments, a subject is a mammal (e.g., a human, a mouse, a cat, a dog, a horse, a hamster, a cow, a pig, or other domesticated animal). In some embodiments, a subject is a human. In some embodiments, a subject is an adult human (e.g., of 18 years of age or older). In some embodiments, a subject is a child (e.g., less than 18 years of age). In some embodiments, a human subject is one who has or has been diagnosed with at least one form of cancer.

In some embodiments, a cancer from which a subject suffers is a carcinoma, a sarcoma, a myeloma, a leukemia, a lymphoma, a melanoma, a mesothelioma, a glioma, or a mixed type of cancer that comprises more than one of a carcinoma, a sarcoma, a myeloma, a leukemia, and a lymphoma. Carcinoma refers to a malignant neoplasm of epithelial origin or cancer of the internal or external lining of the body. Sarcoma refers to cancer that originates in supportive and connective tissues such as bones, tendons, cartilage, muscle, and fat. Myeloma is cancer that originates in the plasma cells of bone marrow. Leukemias (“liquid cancers” or “blood cancers”) are cancers of the bone marrow (the site of blood cell production). Lymphomas develop in the glands or nodes of the lymphatic system, a network of vessels, nodes, and organs (specifically the spleen, tonsils, and thymus) that purify bodily fluids and produce infection-fighting white blood cells, or lymphocytes. Melanoma is a type of skin cancer that originates in the melanocytes of the skin. Mesothelioma's cancers arise from the mesothelium, which forms the lining of organs and cavities, such as, for example, the lungs and the abdomen. Glioma develops in the brain, and specifically in the glial cells, which provide physical and metabolic support to neurons. Examples of a mixed type of cancer include adenosquamous carcinoma, mixed mesodermal tumor, carcinosarcoma, and teratocarcinoma. In some embodiments, a subject has a tumor. A tumor may be benign or malignant.

In some embodiments, a cancer is any one of the following: skin cancer, lung cancer, breast cancer, prostate cancer, colon cancer, pancreatic cancer, rectal cancer, cervical cancer, and cancer of the uterus. In some embodiments, a subject is at risk for developing cancer, e.g., because the subject has one or more genetic risk factors, or has been exposed to or is being exposed to one or more carcinogens (e.g., cigarette smoke, or chewing tobacco).

### Machine Learning

In some embodiments, the techniques developed by the inventors include using one or more trained machine learning models to predict a characteristic of a subject. The machine learning model(s) may include a decision tree model, a gradient boosted decision tree model, a linear regression model, a non-linear regression model (e.g., a logistic regression model), a support vector machine, a Gaussian mixture model, a random forest model, a neural network model, and/or any other suitable type of machine learning model, as aspects of the technology described herein are not limited in this respect. In some embodiments, the machine learning model(s) may include an ensemble of machine learning models of any suitable type (the machine learning models part of the ensemble may be termed “weak learners”).

As described above, in some embodiments, the machine learning model(s) may be implemented as a decision tree classifier. Any suitable type of decision tree classifier may be used and may be trained using any suitable supervised decision tree learning technique. For example, the decision tree classifier may be trained by the iterative dichotomizer technique (e.g., the ID3 algorithm as described, for example, in Quinlan, J. R. 1986. Induction of Decision Trees. Mach. Learn. 1, 1 (March 1986), 81-106)), the C4.5 technique (e.g., as described, for example, in Quinlan, J. R. C4.5: Programs for Machine Learning. Morgan Kaufmann Publishers, 1993), the classification and regression tree (CART) technique (e.g., as described, for example, in Breiman, Leo; Friedman, J. H.; Olshen, R. A.; Stone, C. J. (1984). Classification and regression trees. Monterey, CA: Wadsworth & Brooks/Cole Advanced Books & Software). It should be appreciated that a decision tree classifier may be trained using any other suitable training method, as aspects of the technology described herein are not limited in this respect.

In some embodiments, a gradient-boosted decision tree classifier may be used. The gradient-boosted decision tree classifier may be an ensemble of multiple decision tree classifiers (sometimes called “weak learners”). The prediction (e.g., classification) generated by the gradient-boosted decision tree classifier is formed based on the predictions generated by the multiple decision trees part of the ensemble. The ensemble may be trained using an iterative optimization technique involving calculation of gradients of a loss function (hence the name “gradient” boosting). Any suitable supervised training algorithm may be applied to training a gradient-boosted decision tree classifier including, for example, any of the algorithms described in Hastie, T.; Tibshirani, R.; Friedman, J. H. (2009). “10. Boosting and Additive Trees”. The Elements of Statistical Learning (2nd ed.). New York: Springer. pp. 337-384. In some embodiments, the gradient-boosted decision tree classifier may be implemented using any suitable publicly available gradient boosting framework such as XGBoost (e.g., as described, for example, in Chen, T., & Guestrin, C. (2016). XGBoost: A Scalable Tree Boosting System. In Proceedings of the 22nd ACM SIGKDD International Conference on Knowledge Discovery and Data Mining (pp. 785-794). New York, NY, USA: ACM.). The XGBoost software may be obtained from http://xgboost.ai, for example). Another example framework that may be employed is LightGBM (e.g., as described, for example, in Ke, G., Meng, Q., Finley, T., Wang, T., Chen, W., Ma, W., . . . . Liu, T.-Y. (2017). Lightgbm: A highly efficient gradient boosting decision tree. Advances in Neural Information Processing Systems, 30, 3146-3154.). The LightGBM software may be obtained from https://lightgbm.readthedocs.io/, for example).

In some embodiments, a neural network classifier may be used. The neural network classifier may be trained using any suitable neural network optimization software. The optimization software may be configured to perform neural network training by gradient descent, stochastic gradient descent, or in any other suitable way. In some embodiments, the Adam optimizer (Kingma, D. and Ba, J. (2015) Adam: A Method for Stochastic Optimization. Proceedings of the 3rd International Conference on Learning Representations (ICLR 2015)) may be used.

In some embodiments, a support vector machine (SVM) may be used. The SVM may be implemented using any suitable techniques such as, for example, any of the techniques described by Cristianini, N., and Shawe-Taylor, J. (“An introduction to support vector machines and other kernel-based learning methods.” Cambridge university press, 2000.), which is incorporated by reference herein in its entirety.

In some embodiments, a Gaussian mixture model may be used. The Gaussian mixture model may be implemented using any suitable techniques such as, for example, any of the techniques described by Reynolds, D. (“Gaussian mixture models.” Encyclopedia of biometrics 741.659-663 (2009)), which is incorporated by reference herein in its entirety.

In some embodiments, a random forest model may be used. The random forest model may be implemented using any suitable techniques such as, for example, any of the techniques described by Biau, G. (“Analysis of a random forests model.” The Journal of Machine Learning Research 13.1 (2012): 1063-1095.), which is incorporated by reference herein in its entirety.

### Computer Implementation

An illustrative implementation of a computer system 900 that may be used in connection with any of the embodiments of the technology described herein (e.g., such as the processes of FIG. 2A, FIG. 2B, and FIG. 2C) is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage media 930 in any suitable manner, as the aspects of the technology described herein are not limited to any particular techniques for writing or reading data. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computing system 900 may include a network input/output (I/O) interface 940 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 900 may also include one or more user I/O interfaces 950, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method of predicting a characteristic of a subject based on cell-free RNA (cfRNA) expression data previously-obtained from a biological fluid sample from the subject, the method comprising:
using at least one computer hardware processor to perform:
obtaining the cfRNA expression data; and
processing the cfRNA expression data using a machine learning model trained to process cfRNA expression data from a subject and produce an output indicative of the characteristic of the subject,
wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by:
generating a healthy expression profile component by:
receiving a plurality of RNA expression profiles previously-obtained from biological samples from healthy subjects, the plurality of RNA expression profiles including a respective RNA expression profile for each of one or more cell types and/or each of one or more types of cell-containing samples; and
generating the healthy expression profile component by combining the plurality of RNA expression profiles;
generating a tumor expression profile component; and
generating the artificial cfRNA expression profile by combining the healthy expression profile component and the tumor expression profile component.

2. The method of claim 1, wherein the trained machine learning model is:
a machine learning model that has been trained to predict whether the subject has cancer,
a machine learning model that has been trained to predict whether the subject has liver metastasis,
a machine learning model that has been trained to predict a fraction of malignant B cells relative to total number of B cells in the biological fluid sample from the subject, or
a machine learning model that has been trained to predict a PD-1 status for the subject, wherein the PD-1 status is indicative of whether PDCD1 is expressed in tumor cells of the subject.

3. The method of claim 2, further comprising:
when the trained machine learning model is the machine learning model that has been trained to predict whether the subject has cancer and when the output of the trained machine learning model indicates that the subject has the cancer, generating a recommendation to perform a diagnostic test and/or performing the diagnostic test.

4. The method of claim 3, wherein the cancer is breast cancer or basal breast cancer, and wherein the diagnostic test comprises a mammography and/or a biopsy.

5. The method of claim 2, further comprising:
when the trained machine learning model is the machine learning model trained to predict whether the subject has liver metastasis and when the output of the trained machine learning model indicates that the subject has liver metastasis, (i) generating a recommendation to perform an ultrasound and/or a biopsy, and/or (ii) performing the ultrasound and/or biopsy.

6. The method of claim 2, further comprising:
when the trained machine learning model is the machine learning model that has been trained to predict the fraction of malignant B cells relative to the total number of B cells in the biological fluid sample from the subject, generating a recommendation to administer an anti-cancer treatment based on the fraction of malignant B cells and/or administering the anti-cancer treatment based on the fraction of malignant B cells.

7. The method of claim 6, further comprising determining, based on the fraction of malignant B cells, whether the subject has chronic lymphocytic leukemia (CLL).

8. The method of claim 2, further comprising:
when the trained machine learning model is the machine learning model that has been trained to predict the PD-1 status for the subject, generating a recommendation to administer an anti-cancer treatment based on the PD-1 status and/or administering the anti-cancer treatment based on the PD-1 status.

9. The method of claim 1, wherein the trained machine learning model is a machine learning model that has been trained to predict whether the subject has cancer using training data comprising at least some of the artificial cfRNA expression data including:
a first plurality of artificial cfRNA expression profiles generated using a first plurality of healthy expression profile components, and
a second plurality of artificial cfRNA expression profiles generated using a second plurality of healthy expression profile components and a plurality of tumor expression profile components, the plurality of tumor expression profile components having been generated using tumor expression profiles from tumor samples obtained from subjects having cancer.

10. The method of claim 1, wherein the trained machine learning model is a machine learning model that has been trained to predict whether the subject has liver metastasis using training data comprising at least some of the artificial cfRNA expression data including:
a first plurality of artificial cfRNA expression profiles generated using a first plurality of healthy expression profile components and a first plurality of tumor expression profile components, the first plurality of healthy expression profile components having been generated using at least one RNA expression profile previously-obtained from liver tissue, and
a second plurality of artificial cfRNA expression profiles generated using a second plurality of healthy expression profile components and a plurality of tumor expression profile components, the second plurality of healthy expression profile components having been generated without using at least one RNA expression profile previously-obtained from liver tissue.

11. The method of claim 1, wherein the trained machine learning model is a machine learning model that has been trained to predict a PD-1 status of the subject using training data comprising at least some of the artificial cfRNA expression data including:
a first plurality of artificial cfRNA expression profiles generated using a first plurality of healthy expression profile components and a first plurality of tumor expression profile components, the first plurality of tumor expression profile components having been generated using tumor expression profiles from tumor samples that express PDCD1 (PDCD1+), and
a second plurality of artificial cfRNA expression profiles generated using a second plurality of healthy expression profile components and a second plurality of tumor expression profile components, the second plurality of tumor expression profile components having been generated using tumor expression profiles from tumor samples that do not express PDCD1 (PDCD1−).

12. The method of claim 1, wherein the trained machine learning model is a machine learning model that has been trained to predict a fraction of malignant B cells relative to a total number of B cells in the biological fluid sample from the subject using training data comprising the plurality of artificial cfRNA expression profiles.

13. The method of claim 1, wherein the trained machine learning model is a decision tree model, a gradient boosted decision tree model, a linear regression model, a non-linear regression model, a support vector machine, a Gaussian mixture model, a random forest model, or a neural network model.

14. The method of claim 1, further comprising obtaining the cfRNA expression data from the biological fluid sample from the subject by sequencing the biological fluid sample.

15. The method of claim 1, wherein generating the healthy expression profile component by combining the plurality of RNA expression profiles comprises combining the plurality of RNA expression profiles and a cfRNA expression profile previously-obtained from a biological fluid sample from a healthy subject.

16. The method of claim 1, further comprising training the trained machine learning model to predict the characteristic of the subject using the artificial cfRNA expression data including the plurality of artificial cfRNA expression profiles.

17. The method of claim 16, wherein the plurality of artificial cfRNA expression profiles comprise at least 100 artificial cfRNA expression profiles, at least 250 artificial cfRNA expression profiles, at least 500 artificial cfRNA expression profiles, at least 1,000 artificial cfRNA expression profiles, at least 1,500 artificial cfRNA expression profiles, at least 2,000 artificial cfRNA expression profiles, at least 2,500 artificial cfRNA expression profiles, at least 3,000 artificial cfRNA expression profiles, at least 4,000 artificial cfRNA expression profiles, at least 5,000 artificial cfRNA expression profiles, or at least 10,000 artificial cfRNA expression profiles.

18. The method of claim 1, further comprising generating the artificial cfRNA expression data by generating each particular artificial cfRNA expression profile of the plurality of artificial cfRNA expression profiles.

19. A system, comprising:
at least one computer hardware processor; and
at least one non-transitory computer-readable storage medium storing processor-executable instructions that, when executed by the at least one computer hardware processor, causes the at least one computer hardware processor to perform a method of predicting a characteristic of a subject based on cell-free RNA (cfRNA) expression data previously-obtained from a biological fluid sample from the subject, the method comprising:
obtaining the cfRNA expression data; and
processing the cfRNA expression data using a machine learning model trained to process cfRNA expression data from a subject and produce an output indicative of the characteristic of the subject,
wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by:
generating a healthy expression profile component by:
receiving a plurality of RNA expression profiles previously-obtained from biological samples from healthy subjects, the plurality of RNA expression profiles including a respective RNA expression profile for each of one or more cell types and/or each of one or more types of cell-containing samples; and
generating the healthy expression profile component by combining the plurality of RNA expression profiles;
generating a tumor expression profile component; and
generating the artificial cfRNA expression profile by combining the healthy expression profile component and the tumor expression profile component.

20. At least one non-transitory computer-readable storage medium storing processor-executable instructions that, when executed by at least one computer hardware processor, causes the at least one computer hardware processor to perform a method of predicting a characteristic of a subject based on cell-free RNA (cfRNA) expression data previously-obtained from a biological fluid sample from the subject, the method comprising:
obtaining the cfRNA expression data; and
processing the cfRNA expression data using a machine learning model trained to process cfRNA expression data from a subject and produce an output indicative of the characteristic of the subject,
wherein the machine learning model was trained using artificial cfRNA expression data, the artificial cfRNA expression data comprising a plurality of artificial cfRNA expression profiles, an artificial cfRNA expression profile of the plurality of artificial cfRNA expression profiles having been generated by:
generating a healthy expression profile component by:
receiving a plurality of RNA expression profiles previously-obtained from biological samples from healthy subjects, the plurality of RNA expression profiles including a respective RNA expression profile for each of one or more cell types and/or each of one or more types of cell-containing samples; and
generating the healthy expression profile component by combining the plurality of RNA expression profiles;
generating a tumor expression profile component; and
generating the artificial cfRNA expression profile by combining the healthy expression profile component and the tumor expression profile component.
